(19) United States
(12) Patent Application Publication
(10) Pub. No.: US 2010/0152674 A1
(43) Pub. Date: Jun. 17, 2010

(54) NEEDLE INSERTING AND FLUID FLOW CONNECTION FOR INFUSION MEDIUM DELIVERY SYSTEM

(75) Inventors: Julian D. Kavazov, Arcadia, CA (US); Colin A. Chong, Burbank, CA (US); Rafael Bikovsky, Oak Park, CA (US); Eric M. Lorenzen, Granada Hills, CA (US); Arsen Ibranyan, Glendale, CA (US)

Correspondence Address: FOLEY & LARDNER, 555 South Flower Street, SUITE 3500, LOS ANGELES, CA, 90071-2411, US

(73) Assignee: Medtronic MiniMed, Inc

(21) Appl. No.: 12/597,730

(22) PCT Filed: Aug. 23, 2007
(86) PCT No.: PCT/US07/76679

§ 371 Date: Feb. 1, 2010

Related U.S. Application Data
(60) Provisional application No. 60/927,032, filed on Apr. 30, 2007.

(51) Int. Cl. A61M 5/315; A61M 39/10

(52) U.S. Cl. 604/218; 604/533

(57) ABSTRACT

Needle inserting devices, as well as fluid flow connections and infusion medium delivery systems and methods that may be used with needle inserting devices are described, for medical or non-medical systems, such as, but not limited to sensors, monitors, or the like. The needle inserting device and method may operate to insert a needle or cannula through a patient-user's skin, for example, to provide a fluid flow path for conveying an infusion medium through a hollow channel in the needle or cannula and into the patient-user and/or to convey a fluid from the patient-user to one or more sensor elements. Embodiments of the present invention may be configured, as described herein, to provide a reliable, cost effective and easy-to-use mechanism for inserting a needle or cannula to a specific depth into a patient-user with minimal traumatic effect. In some embodiments, a mechanical force in a first direction results in a needle insertion at a non-zero angle relative to the first direction. In other embodiments, a needle inserter is configured with rotary parts for minimizing the rotation of a needle during insertion.

### CROSS-REFERENCE TO RELATED PATENT APPLICATIONS

This application (National Stage of PCT/US07/076679) claims priority from Provisional Application U.S. Application 60/927,032, filed Apr. 30, 2007, incorporated herein by reference in its entirety. The present invention also relates to U.S. application Ser. No. 11/645,435, filed Dec. 26, 2006, (attorney docket no. 047711-0406); entitled “Infusion Medium Delivery System, Device and Method with Needle Inserter and Needle Inserter Device and Method,” U.S. Provisional Application No. 60/839,840, filed Aug. 23, 2006 (attorney docket no. 047711-0384) and U.S. Provisional Application No. 60/854,829, filed Oct. 27, 2006 (attorney docket no. 047711-0401); each of which is incorporated herein in its entirety. The present invention also relates to U.S. Application No. 60/678,290, filed May 6, 2005 (attorney docket no. 047711-0363) and U.S. application Ser. No. 11/211,095, filed Aug. 23, 2005 (attorney docket no. 047711-0370), entitled “Infusion Device and Method with Disposable Portion,” each of which is incorporated herein by reference in its entirety. The present invention further relates to co-pending U.S. Application No. 60/839, 822, filed Aug. 23, 2006, entitled “Infusion Medium Delivery Device and Method for Driving Plunger in Reservoir” (attorney docket no. 047711-0382); co-pending U.S. Application No. 60/839,832, filed Aug. 23, 2006, entitled “Infusion Medium Delivery Device and Method with Compressible or Curved Reservoir or Conduit” (attorney docket no. 047711-0383); co-pending U.S. Application No. 60/839,741, filed Aug. 23, 2006, entitled “Infusion Pumps and Methods and Delivery Devices and Methods With Same” (attorney docket no. 047711-0385); and co-pending U.S. Application No. 60/839,821, filed Aug 23, 2006, entitled “Systems and Methods Allowing for Reservoir Filling and Infusion Medium Delivery” (attorney docket no. 047711-0381); the contents of each of which is incorporated herein by reference, in its entirety. Embodiments of the present invention also relate to: (i) U.S. application Ser. No. 11/588,832, filed Oct. 27, 2006, entitled “Infusion Medium Delivery Device and Method with Drive Device for Driving Plunger in Reservoir” (attorney docket no. 047711-0387); (ii) U.S. application Ser. No. 11/588,847, filed Oct. 27, 2006, entitled “Infusion Medium Delivery Device and Method with Compressible or Curved Reservoir or Conduit” (attorney docket no. 047711-0390); (iii) U.S. application Ser. No. 11/588,875, filed 10/27/2006, entitled “Systems and Methods Allowing for Reservoir Filling and Infusion Medium Delivery” (attorney docket no. 047711-0393); (iv) U.S. application Ser. No. 11/589,323, filed Aug. 23, 2006, entitled “Infusion Pumps and Methods and Delivery Devices and Methods with Same” (attorney docket no. 047711-0398); (v) U.S. application Ser. No. 11/602,173, filed Nov. 20, 2006, entitled “Systems and Methods Allowing for Reservoir filling and Infusion Medium Delivery” (attorney docket no. 047711-0397); (vi) U.S. application Ser. No. 11/602,052, filed Nov. 20, 2006, entitled “Systems and Methods Allowing for Reservoir filling and Infusion Medium Delivery” (attorney docket no. 047711-0396); (vii) U.S. application Ser. No. 11/602,428, filed Nov. 20, 2006, entitled “Systems and Methods Allowing for Reservoir filling and Infusion Medium Delivery” (attorney docket no. 047711-0395); (viii) U.S. application Ser. No. 11/602,113, filed Nov. 20, 2006, entitled “Systems and Methods Allowing for Reservoir filling and Infusion Medium Delivery” (attorney docket no. 047711-0394); (ix) U.S. application Ser. No. 11/604,172, filed Nov. 22, 2006, entitled “Infusion Medium Delivery Device and Method and Drive Device for Driving Plunger in Reservoir” (attorney docket no. 047711-0389); (x) U.S. application Ser. No. 11/604,171, filed Nov. 22, 2006, entitled “Infusion Medium Delivery Device and Method and Drive Device for Driving Plunger in Reservoir” (attorney docket no. 047711-0388); (xi) U.S. application Ser. No. 11/646,052, filed Dec. 26, 2006, entitled “Infusion Medium Delivery System, Device and Method with Needle Inserter and Needle Inserter Device and Method” (attorney docket no. 047711-0405); (xii) U.S. application Ser. No. 11/645,972, filed Dec. 26, 2006, entitled “Infusion Medium Delivery System, Device and Method with Needle Inserter and Needle Inserter Device and Method” (attorney docket no. 047711-0403); (xiii) U.S. application Ser. No. 11/646,000, filed Dec. 26, 2006, entitled “Infusion Medium Delivery System, Device and Method with Needle Inserter and Needle Inserter Device and Method” (attorney docket no. 047711-0402); (xiv) U.S. application Ser. No. 11/606,836, filed Nov. 30, 2006, entitled “Infusion Pumps and Methods and Delivery Devices and Methods with Same” (attorney docket no. 047711-0400); (xv) U.S. application Ser. No. 11/606,703, filed Nov. 30, 2006, entitled “Infusion Pumps and Methods and Delivery Devices and Methods with Same” (attorney docket no. 047711-0399); (xvi) U.S. application Ser. No. 11/645,993, filed Dec. 26, 2006, entitled “Infusion Medium Delivery Device and Method with Compressible or Curved Reservoir or Conduit” (attorney docket no. 047711-0392); (xvii) U.S. application Ser. No. 11/636,384, filed Dec. 8, 2006, entitled “Infusion Medium Delivery Device and Method with Compressible or Curved Reservoir or Conduit” (attorney docket no. 047711-0391); (xviii) U.S. application Ser. No. 11/515,225, filed Sep. 1, 2006, entitled “Infusion Medium Delivery Device and Method with Drive Device for Driving Plunger in Reservoir” (attorney docket no. 047711-0386); the contents of each of which are incorporated by reference herein, in their entirety.

### FIELD OF THE INVENTION

Embodiments of the present invention relate to needle inserting devices, reservoir filling arrangements, bubble management, fluid flow connections and infusion medium delivery systems and methods that employ the same. Further embodiments relate to the needle inserting devices and methods for or included in other types of medical or non-medical systems, such as, but not limited to sensors, monitors, or the like.

### BACKGROUND

Certain chronic diseases may be treated, according to modern medical techniques, by delivering a medication or other substance to a patient-user's body, either in a continuous manner or at particular times or time intervals within an overall time period. For example, diabetes is a chronic disease that is commonly treated by delivering defined amounts of insulin to the patient-user at appropriate times. Some common modes of providing an insulin therapy to a patient-user include delivery of insulin through manually operated syringes and insulin pens. Other modern systems employ programmable pumps to deliver controlled amounts of insulin to a patient-user.

Pump type delivery devices have been configured in external devices (that connect to a patient-user) or implantable devices (to be implanted inside of a patient-user's body). External pump type delivery devices include devices designed for use in a generally stationary location (for example, in a hospital or clinic), and further devices configured for ambulatory or portable use (to be carried by a patient-user). Examples of some external pump type delivery devices are described in U.S. patent application Ser. No. 11/211,095, filed Aug. 23, 2005, titled “Infusion Device And Method With Disposable Portion” and Published PCT Application WO 01/70307 (PCT/US01/09139) titled “Exchangeable Electronic Cards For Infusion Devices” (each of which is owned by the assignee of the present invention), Published PCT Application WO 04/030716 (PCT/US2003/028769) titled “Components And Methods For Patient Infusion Device,” Published PCT Application WO 04/030717 (PCT/US2003/029019) titled “Dispenser Components And Methods For Infusion Device,” U.S. Patent Application Publication No. 2005/0065760 titled “Method For Advising Patients Concerning Doses Of Insulin,” and U.S. Pat. No. 6,589,229 titled “Wearable Self-Contained Drug Infusion Device,” each of which is incorporated herein by reference in its entirety.

External pump type delivery devices may be connected in fluid-flow communication to a patient-user, for example, through a suitable hollow tubing. The hollow tubing may be connected to a hollow needle that is designed to pierce the patient-user's skin and deliver an infusion medium to the patient-user. Alternatively, the hollow tubing may be connected directly to the patient-user as or through a cannula or set of micro-needles.

In contexts in which the hollow tubing is connected to the patient-user through a hollow needle that pierces the patient-user's skin, a manual insertion of the needle into the patient-user can be somewhat traumatic to the patient-user. Accordingly, insertion mechanisms have been made to assist the insertion of a needle into the patient-user, whereby a needle is forced by a spring to quickly move from a refracted position into an extended position. As the needle is moved into the extended position, the needle is quickly forced through the patient-user's skin in a single, relatively abrupt motion that can be less traumatic to certain patient-users as compared to a slower, manual insertion of a needle. While a quick thrust of the needle into the patient-user's skin may be less traumatic to some patient's than a manual insertion, it is believed that, in some contexts, some patients may feel less trauma if the needle is moved a very slow, steady pace. Examples of insertion mechanisms that may be used with and may be built into a delivery device are described in: U.S. patent application Ser. No. 11/645,435, filed Dec. 26, 2006, titled “Infusion Medium Delivery system, Device And Method With Needle Inserter And Needle Inserter Device And Method,”; and U.S. patent application Ser. No. 11/211,095, filed Aug. 23, 2005, titled “Infusion Device And Method With Disposable Portion” (each of which is assigned to the assignee of the present invention), each of which is incorporated herein by reference in its entirety. Other examples of insertion tools are described in U.S. Patent Application Publication No. 2002/0022855, titled “Insertion Device For An Insertion Set And Method Of Using The Same” (assigned to the assignee of the present invention), which is incorporated herein by reference in its entirety. Other examples of needle/cannula insertion tools that may be used (or modified for use) to insert a needle and/or cannula, are described in, for example U.S. patent application Ser. No. 10/389,132 filed Mar. 14, 2003, and entitled “Auto Insertion Device For Silhouette Or Similar Products,” and/or U.S. patent application Ser. No. 10/314,653 filed Dec. 9, 2002, and entitled “Insertion Device For Insertion Set and Method of Using the Same,” both of which are incorporated herein by reference in their entirety.

As compared to syringes and insulin pens, pump type delivery devices can be significantly more convenient to a patient-user, in that accurate doses of insulin may be calculated and delivered automatically to a patient-user at any time during the day or night. Furthermore, when used in conjunction with glucose sensors or monitors, insulin pumps may be automatically controlled to provide appropriate doses of infusion medium at appropriate times of need, based on sensed or monitored levels of blood glucose.

Pump type delivery devices have become an important aspect of modern medical treatments of various types of medical conditions, such as diabetes. As pump technologies improve and doctors and patient-users become more familiar with such devices, the popularity of external medical infusion pump treatment increases and is expected to increase substantially over the next decade.

### BRIEF DESCRIPTION OF THE DRAWINGS

FIGS. 1-10 illustrate various aspects of a multiple-septum connections arrangement.

FIGS. 11-13 illustrate an example of an arrangement for connecting a drive shaft to a piston plunger in a pump device.

FIGS. 14-21 illustrate examples of a rotary needle inserting device.

FIGS. 22-70 illustrate further examples of needle inserting devices.

FIG. 71 illustrates a skin spreader arrangement.

FIGS. 72 and 73 illustrate an infusion medium delivery system with a injection site module.

FIG. 74 illustrates an adhesive patch in accordance with an embodiment of the present invention;

FIGS. 75-79 illustrate various tubing connector arrangements.

### DETAILED DESCRIPTION

Aspects of the present invention relate, generally, to needle inserter or inserting devices and methods and medical devices, such as, but not limited to sensors, monitors and infusion medium delivery systems, devices and methods that include such needle inserting devices and methods. The needle inserting device and method may operate to insert a needle or cannula through a patient-user's skin, for example, to provide a fluid flow path for conveying an infusion medium through a hollow channel in the needle or cannula and into the patient-user and/or to convey a fluid from the patient-user to one or more sensor elements. Embodiments of the present invention may be configured, as described herein, to provide a reliable, cost effective and easy-to-use mechanism for inserting a needle or cannula to a specific depth into a patient-user with minimal traumatic effect.

In addition, embodiments may be configured to establish a contiguous fluid-flow passage for fluid transfer between a reservoir and the patient-user, when the hollow needle or cannula is inserted into the patient-user. Needle inserting devices according to embodiments of the present invention may be used with, connectable to and disconnectable from or incorporated in a portion of an infusion medium delivery system. For example, a needle inserting device may be connectable to a base structure of a pump type delivery device for insertion of a needle, after which the needle inserting device may be removed from the base structure, whereupon a further housing portion of the delivery device (containing components such as, but not limited to, a reservoir and pump or drive device) may be coupled to the base structure for operation. Alternatively, the needle inserting device may be incorporated into the further housing portion that contains other components as described above. In yet other embodiments, the needle inserting device may be connectable to (and releasable from) or incorporated within an injection site module or other housing that connects, for example, by flexible tubing, to other components of a medical device (such as, but not limited to an infusion medium delivery device). In yet other embodiments, needle inserter devices may be configured for use with systems other than infusion medium delivery systems, such as, but not limited to sensor and monitor systems, or the like.

Further aspects of the present invention relate to reservoir filling systems and processes and bubble management systems and processes for controlling bubbles during filling of a reservoir or operation of an infusion medium delivery device. Yet further aspects of the invention relate to connection structures for connecting devices in fluid-flow communication and tubing connectors that may be used for connecting fluid conduits used in infusion medium delivery devices or other systems involving fluid-flow.

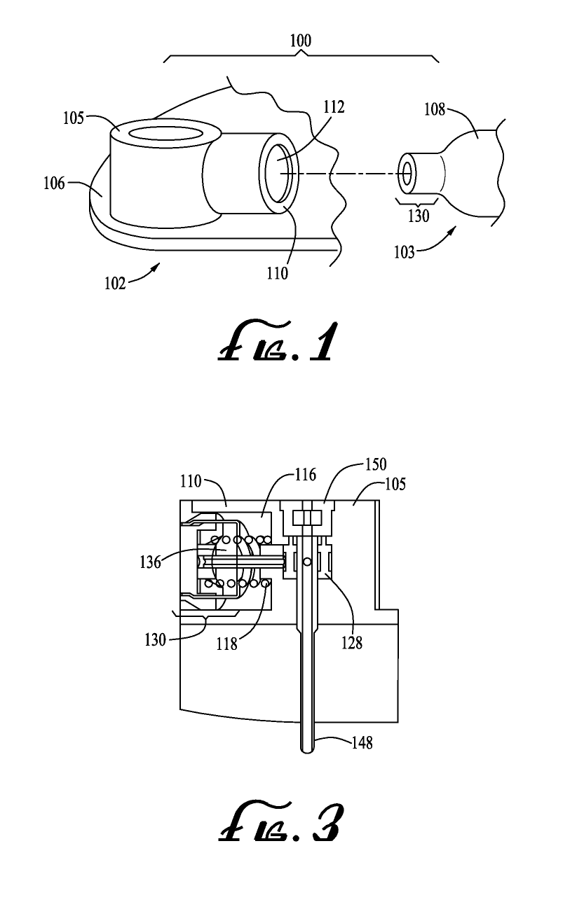

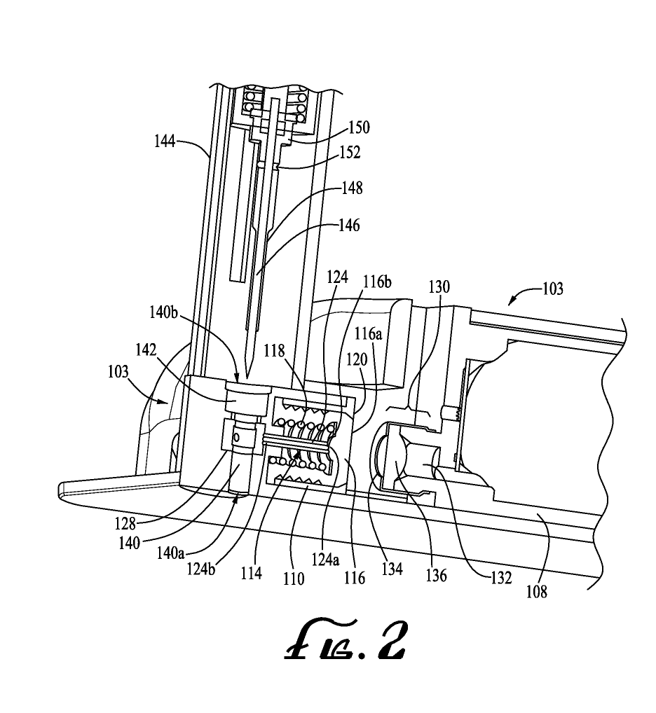

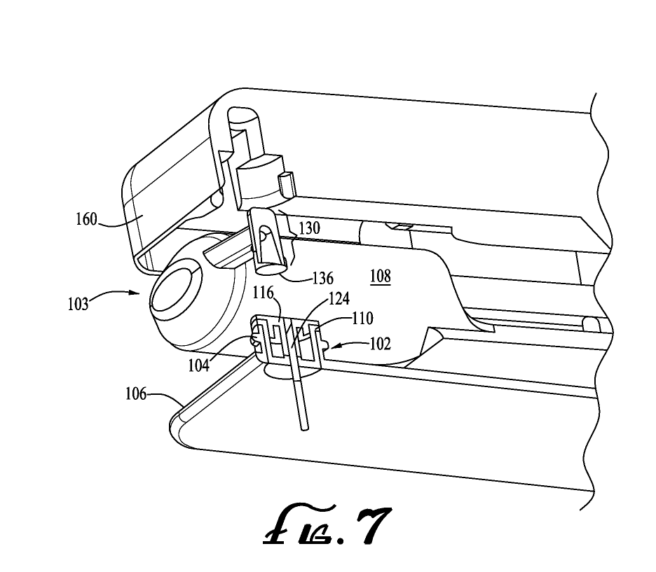

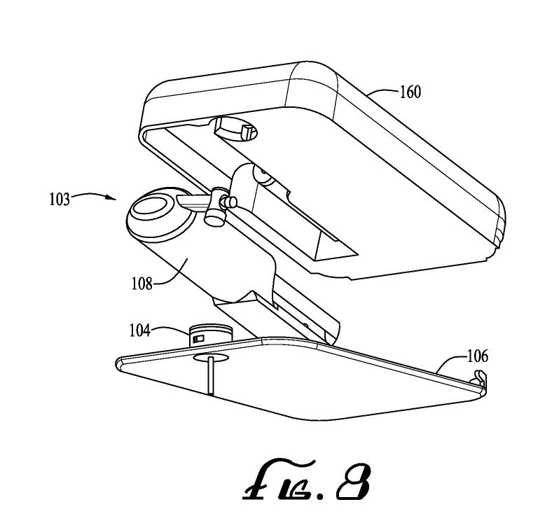

### Embodiment of FIGS. 1-6

A structure and method for connecting two members in fluid flow communication is described with reference to FIGS. 1-6.

The structure and method described with respect to FIGS. 1-6 may be employed in any suitable device or system in which two members that, at some period of time, are not connected in fluid flow communication, are to be connected together in a manner that allows fluid to flow from one member to the other. In one example embodiment, the structure and method is described with respect to a first member including a fluid reservoir for containing an infusion medium that is connectable to a second member including an injection site structure in which a hollow needle or cannula is or may be inserted into a patient-user, for conveying fluid media to the patient-user. However, connection structure according to embodiments of the present invention may be employed to connect any two (or more) members together, for fluid flow communication with each other.

In FIGS. 1-6, an example of a structure 100 and method for connecting two members in fluid flow communication is described with reference to a first member 102 and a second member 103. The first member 102 in the illustrated example includes a housing 104 on a base 106. The housing 104 may be formed integral with the base 106 or may be formed as a separate structure that is connected to the base 106 in a fixed relation to the base 106. The housing 104 and base 106 each may be made of any suitably rigid material, including, but not limited to plastic, metal, ceramic, composite material or the like.

The housing 104 in the illustrated example includes a section 105 that contains an injection site structure, in which a hollow needle or cannula may be inserted into a patient-user for conveying fluidic media to or from the patient-user. In other embodiments, instead of or in addition to an injection site, the housing 104 may contain, be part of or be operatively connected to any other suitable structure for conveying, containing and/or processing a fluidic medium.

The second member 103 also includes a housing 108, which, in the illustrated embodiment, is a housing of a reservoir for containing an infusion media. The second member 103 may be held within or otherwise covered by a further housing member 109 that is configured to attach to the base 106. The further housing 109 may connect to the base 106 of the first member 102 by any suitable connection structure. In particular embodiments, one or other of the housing 109 and the base 106 may include one or more flexible pawls, protrusions and/or indentations for engaging and receiving one or more corresponding pawls, protrusions and/or indentations on the other of the base 106 and the housing 109, to provide a suitable connection structure. Alternatively or in addition, the connection structure may include adhesive material or other suitable connectors.

In other embodiments, the housing 108 may be (or be connected to) a sensor housing that contains sensor components. In yet other embodiments, the housing 108 may contain, be part of or be operatively connected to any other suitable structure for conveying, containing and/or processing a fluidic medium. The housing 108 may be made of any suitably rigid material, including, but not limited to plastic, metal, ceramic, composite material or the like.

The housing 104 has or is connected to a receptacle structure 110. The receptacle structure has an opening 112 in the housing, that leads into a chamber 114 within the receptacle structure. In the illustrated embodiment, the receptacle structure 110 is part of the housing 104, adjacent the section of the housing that contains the injection site. In other embodiments, the receptacle structure 110 may include a further housing that is connected to the housing 104.

The receptacle structure 110 includes a first septum 116 located within the chamber 114 and moveable within the chamber 114, toward and away from the opening 112. The receptacle structure 110 also includes a bias mechanism 118, that applies a bias force on the septum 116, in the direction toward the opening 112. The bias mechanism 118 may force the septum 116 against the opening 112, wherein an annular protrusions (or one or more appropriately shaped or positioned protrusions) 120 adjacent the opening 112 may be provided to inhibit the septum 116 from being forced out of the chamber 114, through the opening 112. The septum 116 has a front surface 116a that is at least partially exposed through the opening 112, when the septum 116 is urged against the opening 112 by the bias mechanism 118. The septum 116 has a back surface 116b that faces toward the interior of the chamber 114. The septum 116 may be made of any suitable material that may be pierced by the needle 124, such as, but not limited to a natural or synthetic rubber material, silicon or the like. In particular embodiments, the septum 116 may be made of a self sealing material that is capable of sealing itself after a needle has pierced the septum and was subsequently withdrawn from the septum.

In the illustrated embodiment, the bias mechanism 118 is a coil spring located within the chamber 114, on the opposite side of the septum 116 with respect to the side of the septum that faces the opening 112. In other embodiments, the bias mechanism 118 may be provided by other suitable means for biasing the septum 116 toward the opening 112, including, but not limited to, other types of springs, pressurized fluid within the chamber 114, a collapsible skirt structure 122 extending from the septum 116 that has a natural or built-in spring force, chemical or substance that expands upon contact with another chemical or substance or upon application of energy from an energy source such as a heat, laser or other radiation source, or the like.

A hollow needle 124 is supported within the chamber 114, with a sharp end 124a of the needle 124 directed toward the back surface 116b of the septum 116. In the illustrated embodiment, the hollow needle 124 is supported within the coil spring bias mechanism 118, with its longitudinal axial dimension extending generally parallel to the longitudinal axial dimension of the coil spring. The hollow needle 124 may be supported by a supporting structure 126 located within the receptacle structure. In the illustrated embodiment , the supporting structure 126 is a wall that is integral with the housing of the receptacle structure 110 and is located on the opposite end of the chamber 114 relative to the end of the chamber 114 at which the opening 112 is located. However, in other embodiments, the supporting structure 126 may be any suitable structure that is generally fixed relative to the housing of the receptacle structure 110 and is able to support the needle 124 in a generally fixed relation to the housing of the receptacle structure 110.

The hollow needle 124 may be made of any suitably rigid material, including, but not limited to metal, plastic, ceramic, or the like, and has a hollow channel that extends in a lengthwise dimension of the needle. The hollow channel in the needle 124 is open on the sharp end 124 of the needle and is open at another location 124b along the length of the needle, such as, but not limited to, the needle end that is opposite to the sharp end 124a. The hollow channel in the needle 124 provides a fluid flow path between the sharp end 124a of the needle and the opening 124b of the needle. In the illustrated embodiment, the opening 124b of the hollow needle 124 is connected in fluid flow communication with a manifold 128 in a needle injector structure described below.

The housing 108 of the second member 103 includes a connection portion 130 that has a hollow interior chamber 132 and an opening 134 into the hollow interior. A second septum 136 is supported by the housing 108 to seal the opening 134. The septum 136 may be supported in a fixed relation to the housing 108, for example, within housing 108, at one end of the chamber 132.

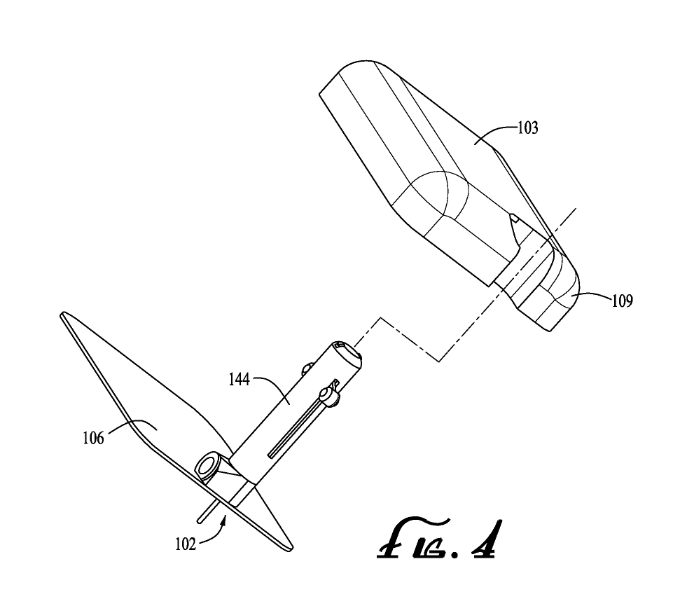

The connection portion 130 of the housing 108 has a suitable shape and size to fit at least partially within the opening 112 of the receptacle structure 110 in the first member 102, when the first and second members 102 and 103 are connected together. In the drawings of FIGS. 1 and 2, the first and second members 102 and 103 are shown in a separated, disconnected relation, wherein the connection portion 130 of the housing 108 is outside of the opening 112 of the receptacle structure 110. By moving the first and second members 102 and 103 together to insert the connection portion 130 into the opening 112 of the housing 108, an end surface 138 of the connection portion 130 is urged against the moveable septum 116 and causes the moveable septum 116 to move relative to the housing 108, against the force of the bias mechanism 118, toward the interior of the chamber 114. As the septum 116 is moved toward the interior of the housing 108, the sharp end 124a of the needle 124 pierces the septum 116. Continued relative movement of the first and second members 102 and 103 together causes the sharp end 124a of the needle 124 to pass through the septum 116 in the first member 102 and then pierce and pass through the septum 136 in the second member 103.

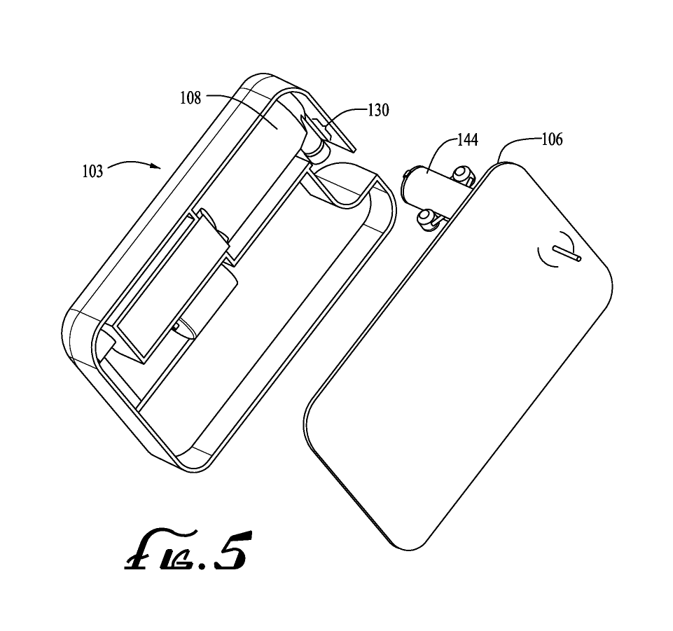

When the first and second members 102 and 103 are brought together as described above and as shown in FIG. 3, at least a portion of the connection portion 130 extends inside of the housing of the receptacle structure 110. In addition, the hollow needle pierces the first and second septa 116 and 136 to form a fluid flow path between the interior chamber 132 of the connection portion 130 and the manifold 128 (or other structure at the opening 124b of the needle 124). The receptacle structure 110 and the connection portion 130 may be provided with mating connectors that provide, for example, a snap or friction connection, upon the first and second members 102 and 103 being brought together as shown in FIG. 3. In one embodiment, the mating connectors may include a protrusion on one or the other of the receptacle structure 110 and the connection portion 130 and a groove or indentation in the other of the receptacle structure 110 and the connection portion 130, arranged to engage each other in a snap-fitting manner, upon the connection portion 130 being extending into the receptacle structure 110 a suitable distance.

As mentioned above, in the illustrated embodiment, the opening 124b of the needle 124 is connected in fluid flow communication with the manifold 128 in an injection site structure. The injection site structure is provided within the section 105 of the housing 104 (FIG. 1) and includes a channel 140 that extends through the housing 104 and the base 106. The channel 140 has an open end 140a on the bottom surface (relative to the orientation shown in FIG. 2) of the base 106. The channel 140 has another open end 140b at the upper surface (relative to the orientation shown in FIG. 2) of the section 105 of the housing 104. The manifold 128 is located along the length of the channel 140 and is in fluid flow communication with the channel 140. Accordingly, the hollow needle 124 is arranged in fluid flow communication with the interior of the channel 140, through the manifold 128. The channel 140 includes a channel section 142 that has a larger radial dimension relative to the rest of the channel 140 and has a suitable shape and size to receive a cannula head, as described below.

A needle inserting device 144 may be located adjacent the open end 140b of the channel 140 and arranged to selectively extend a needle and/or cannula into the open end 140b of the channel and at least partially through the channel 140 as described below. The needle inserting device 144 may be configured to be integral with or otherwise fixed to the section 105 of the housing 104 of the first member 102. Alternatively, the needle inserting device 144 may be a separate device (relative to the housing 104) and may be selectively connected to (in alignment with the channel 140 as shown in FIG. 2) and disconnected from the section 105 of the housing 104.

In embodiments in which the needle inserting device 144 is a separate structure that connects to and disconnects from the housing section 105, suitable connection structure may be provided on the needle inserting device 144 and the housing section 105 to provide a manually releasable connection between those components. Such connection structure may include, but not limited to a threaded extension on one or the other of the needle inserting device 144 and the housing section 105 and a corresponding threaded receptacle on the other of the housing section 105 and the needle inserting device 144, for receiving the threaded extension in threaded engagement. In other embodiments, other suitable connection structure may be employed, including, but not limited to flexible pawls or extensions on one or the other of the needle inserting device 144 and the housing section 105 and a corresponding aperture, stop surface or the like on the other of the other of the housing section 105 and the needle inserting device 144.

In the drawing of FIG. 2, the needle inserting device 144 is shown as connected to the housing section 105 and with a needle 146 and cannula 148 in a retracted state. The needle inserting device 144 operates to selectively move the needle 146 and cannula 148 from the retracted state (shown in FIG. 2) to an extended state (not shown) in which the needle and cannula are extended through the opening 140b of the channel 140 and at least partially through the channel 140, such that the sharp end of the needle 146 and at least a portion of the length of the cannula 148 extend out the opening 140a of the channel 140. Various examples of suitable structure for needle inserting devices are described in U.S. patent application Ser. No. 11/645,435, filed Dec. 26, 2006, (attorney docket no. 047711.0406), titled “Infusion Medium Delivery system, Device And Method With Needle Inserter And Needle Inserter Device And Method,” which is assigned to the assignee of the present invention and is incorporated herein by reference, in its entirety. Other examples of suitable structure for needle inserting devices are described herein.

The cannula 148 has a hollow central channel extending along its longitudinal length and open at one end (the cannula end adjacent the sharp end of the needle 146). The other end of the cannula 148 has a head 150 that has a larger radial dimension than the shaft portion of the cannula. The cannula head 150 has a suitable shape and size to fit into the section 142 of the channel 140, when the needle 146 and cannula 148 are moved to the extended state by the needle inserting device 144. In particular embodiments, the cannula head 150 may include one or more protrusions and/or indentations that engage with one or more corresponding indentations and/or protrusions in the channel section 142 of the housing section 105, to provide a friction fit, snap fit or the like, to lock or retain the cannula 148 in place within the housing section 105, upon the needle 146 and cannula 148 being moved to the extended state by the needle inserting device 144. In further embodiments, instead of or in addition to engaging protrusions and indentations, other mechanical structure may be employed to provide a suitable retaining function for retaining the cannula 148 in place within the housing section 105, upon the needle 146 and cannula 148 being moved to the extended state by the needle inserting device 144, including but not limited to friction fit structure, snap fit, or the like.

The cannula 148 also has a connection channel 152 that is provided in fluid flow communication with the central, longitudinal channel of the cannula. The connection channel 152 is provided, along the longitudinal length of the cannula, at a location at which the channel 152 aligns with the manifold 128 (in fluid flow communication with the interior of the manifold 128), when the needle 146 and cannula 148 have been moved to the extended state by the needle inserting device 144. In this manner, upon the cannula 148 being moved to the extended state, the central, longitudinal channel of the cannula is arranged in fluid flow communication with the hollow needle 124, through the manifold 128 and connection channel 152.

Accordingly, in operation, a first member 102 (which may include, for example, a housing 104 that has a receptacle 110 and a injection site section 105) is coupled together with a second member 103 (which may include, for example, a fluid reservoir housing 108), by inserting the connection portion 130 of the second member 103 into a receptacle 110 of the first member 102. Upon coupling the first and second members 102 and 103, fluid flow communication is provided between the interior of the second member 103 and the injection site structure in the first member 102.

In addition, the needle inserting device 144 is coupled to the section 105 of the housing 104 of the first member 102 (or is provided as part of a single, unitary structure with the section 105 of the housing 104). The base 106 of the first member 102 may be secured to a patient-user's skin (at a suitable injection location) with, for example, but not limited to, adhesive material as described in U.S. patent application Ser. No. 11/645,435, filed Dec. 26, 2006, (attorney docket no. 047711.0406), titled “Infusion Medium Delivery system, Device And Method With Needle Inserter And Needle Inserter Device And Method,” and/or as described herein. Alternatively or in addition, the base 106 may be secured to a patient-user by other suitable structure, including, but not limited to straps, or the like.

Once the base is suitably secured to the patient-user's skin at a suitable injection location, the inserting device 144 may be actuated to move the needle 146 and cannula 148 from a retracted state (shown in FIG. 2), to an extended state. In the extended state, the needle 146 and cannula 148 pierce the patient-user's skin adjacent the base 106. The cannula 148 may be locked into its extended state by engagement of the cannula head 150 and the channel section 142, as described above. With the cannula 148 locked in the extended state, the needle 146 may be retracted (for example, by automatic operation of the needle inserting device 144 and/or by manual removal of the needle inserting device 144 from the housing section 105). Once the needle 146 is removed, the cannula 148 is held in place by the housing section 105, with a portion of the cannula 148 extending into the patient-user, and with the cannula 148 connected in fluid-flow communication with the hollow needle 124. If the first and second members 102 and 103 are connected together, as described above, then a fluid-flow connection is provided from the reservoir 108 to the cannula 148, through the hollow needle 124 and the manifold 128.

The connection sequence (e.g., the sequence of connecting the needle inserting device 144 to the section 105 of the housing 104, connecting the receptacle 110 of the housing 104 to the connection portion 130 of the reservoir housing 108, and connecting the base 106 of the first member to a patient-user's skin) may be different for different embodiments. In one embodiment, a patient-user may be provided with a first member 102 that includes the base 106 and the housing 104 (including housing portion 105) in a pre-connected state with the needle inserting device 144. In this manner, the patient-user need not have to connect the needle inserting device 144 to the housing 104 (as those parts are supplied to the user in a pre-connected state, for example, from a manufacturing or assembly facility). In that embodiment, the patient-user (or a medical practitioner) may secure the base 106 of the first member 102 to his or her skin, at a suitable injection location. After securing the base 106 to the patient-user's skin, the patient-user (or a medical practitioner) may activate the needle inserting device 144 to cause the needle 146 and cannula 148 to be moved to the extended state and pierce the patient-user's skin.

After activation of the needle inserting device 144, the needle inserting device 144 may be removed from the housing section 105, leaving the cannula 148 in place within the housing section 105 and partially extended into the patient-user. With the base 106 of the first member 102 secured to the patient-user's skin and the cannula 148 inserted at least partially into the patient-user and arranged in fluid-flow communication with the hollow needle 124, the second member 103 may be connected to the first member 102. In particular, the connection portion 130 of the housing 108 of the second member 103 may be inserted into the receptacle 110 of the housing 104 of the first member 102, to provide a fluid-flow connection between the interior of the housing 108 and the hollow needle 124 and, thus, the cannula 148. Accordingly, the interior of the housing 108 (which may be a reservoir housing) may be coupled in fluid flow communication with a cannula 148 that has been extended into a patient-user, for delivering fluid from the reservoir, to the patient-user (or for conveying fluid from the patient-user to the reservoir).

While the connection sequence in the above embodiment involves securing the base 106 of the first member 102 to the patient-user, prior to connection of the second member 103 to the first member 102, in other embodiments, the second member 103 may be connected to the first member 102 (as described above) prior to securing the base 106 of the first member onto a patient-user's skin. In such other embodiments, the first and second members 102 and 103 may be connected together and, thereafter, the connected members 102 and 103 may be secured to a patient-user by adhering one or both of the first and second members 102 and 103 to the patient user's skin. Also, while the connection sequence in the above embodiment involves activating the needle inserting device prior to the connection of the second member 103 to the first member 102, in other embodiments, the second member 103 may be connected to the first member 102 (as described above) prior to activating the needle inserting device 144.

In the embodiment shown in FIGS. 1 and 2, the receptacle 110 is in the first member 102 and the connection portion 130 is in the second member 103. However, in other embodiments, the receptacle 110 may be in the second member 103 (for example, in or associated with a housing for a reservoir 108) and the connection portion 130 may be in the first member 102 (for example, in or associated with a housing that contains an injection site structure). Also, in the embodiment shown in FIGS. 1 and 2, the receptacle 110 is arranged to allow the connection portion 130 of the second member 103 to be inserted in a direction substantially parallel to the plane of the upper-facing (in the orientation of FIG. 2) surface of the base 106. In the orientation of FIG. 2, this direction of insertion is shown as a horizontal direction of relative motion between the first and second members 102 and 103. However, in other embodiments, the receptacle 110 may be arranged in other suitable orientations, including, but not limited to an orientation that allows an insertion direction (relative motion of the first and second members 102 and 103) to be substantially perpendicular to the plane of the upper-facing (in the orientation of FIG. 2) surface of the base 106. In yet other embodiments, the receptacle 110 may be arranged to allow any other suitable insertion direction at an angle transverse to the plane of the upper-facing (in the orientation of FIG. 2) surface of the base 106.

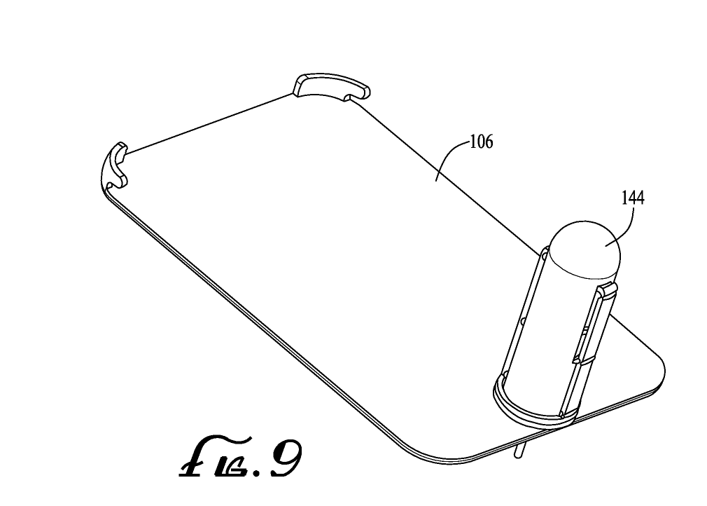

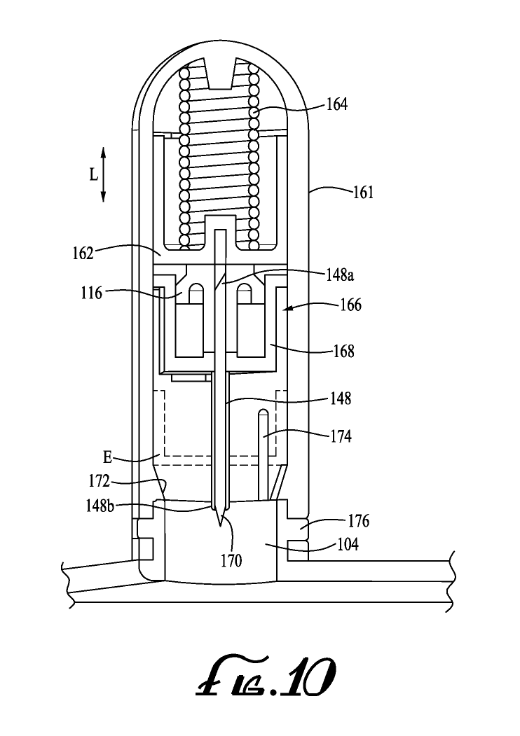

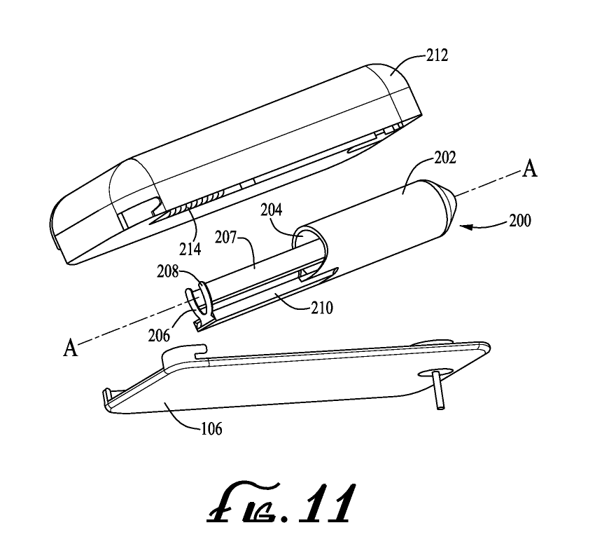

An example arrangement shown in FIGS. 7-10 provides an insertion direction (relative motion of the first and second members 102 and 103) that is substantially perpendicular to the plane of the upper-facing (in the orientation of FIG. 2) surface of the base 106. Components in FIGS. 7-10 are identified by reference numbers that are the same reference numbers used in FIGS. 1-6 for components having similar structure and function. In FIGS. 7 and 8, the injection site structure in the housing 104 is shown in a state after a needle inserting device has been operated to move a cannula 148 to the extended position.

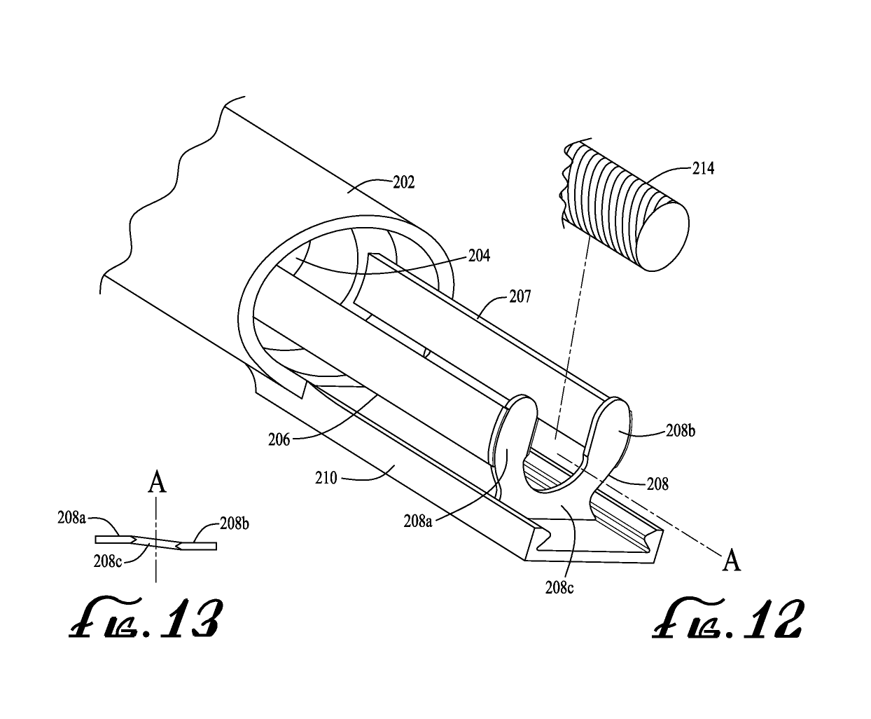

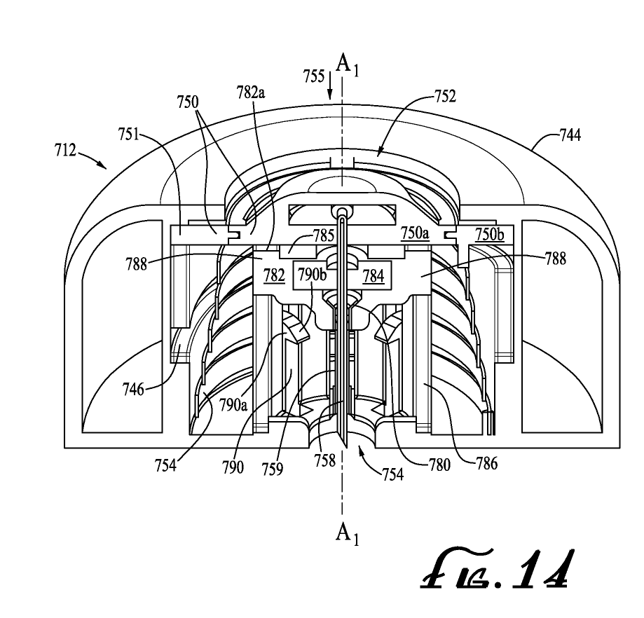

FIGS. 9 and 10 show the base 106 of the first member 102 (of the embodiment of FIGS. 7 and 8) with a needle inserting device 144 attached to the housing 104. The needle inserting device 144 in FIGS. 9 and 10 includes a housing 160 that is securable to the base 106 in any suitable manner, such as, but not limited to the manners of connecting an inserting device 144 to the housing 105 discussed above with respect to the embodiment of FIGS. 1-6. As shown in FIG. 10, the housing 160 contains an internal chamber having a longitudinal dimension L and a moveable plunger 162 located within the housing 160 and moveable along the longitudinal dimension L, from a retracted position (shown in solid lines in FIG. 10) to an extended position (in which the plunger 162 is moved to a position E shown in broken lines in FIG. 10). A bias member 164, such as, but not limited to, a coil spring arranged within the housing 160, imparts a bias force on the plunger, when the plunger is in the retracted position, to urge the plunger 162 toward the extended position E. A locking mechanism (not shown) may be provided such as, but not limited to, a manually moveable projection, lever or slider that is connected to or extends through the housing 160 and engages the plunger 162 (or other structure holding the plunger) in a releasable manner, to selectively hold the plunger 162 in its retracted state, against the bias force of the bias member 164 and to allow a user to selectively release the plunger to move in the longitudinal direction L under the force of the bias member 164.

An insert structure 166 is arranged within the housing 160 for movement in the longitudinal direction L by action of movement of the moveable plunger 162. The insert structure 166 includes a cup-shaped body 168 that holds a first septum 116 (similar to the septum 116 described above with respect to the embodiment of FIGS. 1-6). A hollow cannula 148 (similar to the cannula 148 described above) has one open end 148a that may have a sharp tip positioned adjacent the septum 116 (or at least partially within the septum 116). The hollow cannula 148 extends through the cup-shaped body 168 and has a second open end 148b. The hollow cannula 148 may be fixed to the cup-shaped member 168, to move with movement of the cup-shaped member 168. A needle 170 is secured to the plunger 162 and extends through the septum 116 and cannula 148, when the plunger 162 is in the retracted position shown in FIG. 10.

In operation, a patient-user (or medical practitioner) may secure the base 106 to a patient-user's skin (as described above with respect to base 106 in FIGS. 1-6). Once the base 106 is secured to the patient-user's skin, the patient-user (or medical practitioner) may activate the needle inserting device 144 to cause the plunger 162 to move from its retracted state to its extended state and, as a result of such movement, to cause the insert structure 166 to be moved into the an opening into the interior of the housing 104. Upon movement of the insert structure 166 into the housing 104, the insert structure 166 may connect to the base housing 104 by any suitable connection structure. In particular embodiments, one or other of the cup-shaped member 168 of the insert structure 166 and the housing 104 may include one or more flexible pawls, protrusions and/or indentations for engaging and receiving one or more corresponding pawls, protrusions and/or indentations on the other of the housing 104 and the insert structure 166, to provide a suitable connection structure. Alternatively or in addition, the connection structure may include adhesive material or other suitable connectors. FIG. 7 shows the insert structure 166 in the extended position, and locked into the housing 104 (e.g., after insertion by the inserting device 144 and after removal of the inserting device 144 from the housing 104).

In particular embodiments, the housing 160 of the needle inserting device 144 may automatically release from the base 106, upon movement of the plunger 162 and the insert structure 166 from the retracted state (shown in FIG. 10) to an extended state. For example, the housing 160 of the needle inserting device 144 may be made of a material that has sufficient rigidity to operate as described herein, but also has a suitable flexibility (at least at the portion of the device 144 that connects to the housing 104) to bend away from and release from the housing 104, upon movement of the insert structure 166 to the extended state.

As shown in FIG. 10, a portion 172 of the internal surface of the housing 160 may include a ramped, wedge-shaped or angled (relative to an axial direction of the housing 144, cannula 148 and needle 170) cross-sectional shape that engages an outer peripheral surface of the insert structure 166 and/or the plunger 162, as the insert structure 166 and plunger 162 are moved toward the extended state. By engaging the angled, ramped or wedge-shaped portion 172 of the internal surface of the housing 160, the plunger 162 and/or insert structure 166 causes the wall(s) of the housing 160 to flex outward, as the plunger 162 and insert structure 166 are moved into the extended position. One or more slots, grooves or the like 174 may be formed in the housing 166 to enhance the ability of the wall(s) of the housing 160 to flex outward. One or more protrusions 176 and/or indentations may be provided on one or the other of the interior surface of the housing 166 and the exterior surface of the housing 104 for engaging one or more corresponding indentations 178 and/or protrusions in the other of the housing 104 and housing 166, when the plunger 162 and insert structure 166 are in the retracted state shown in FIG. 10.

The protrusions 176 and indentations 178, when engaged, lock the housing 160 of the needle inserting device 144 to the housing 104. The one or more protrusions and/or indentations disengage from each other, when the wall(s) of the housing 160 are flexed outward by the movement of the plunger 162 and insert structure 166 to the extended state. As a result, the housing 160 of the needle inserting device 144 may be automatically disengaged and released from the housing 104, upon movement of the plunger 162 and insert structure 166 to the extended state. After movement of the plunger 162 and insert structure 166 from the retracted state (shown in FIG. 10) to the extended state (at which the insert structure 166 will be locked into the housing 104, while the housing 166 of the needle inserting device is released from the housing 104), the bias member 164 (or a second bias member, not shown) may act on the needle 170 to move the needle 170 toward the retracted position and, thus, withdraw the needle 170 from the cannula 148. For example, a return motion of the coil spring after moving from the retracted state to the extended state may provide sufficient force to withdraw the needle 170 from the cannula 148.

Once the insert structure 166 has been locked into place within the housing 104 and the needle inserting device 144 removed from the housing 104, the cannula 148 may be connected in fluid flow communication with a connection portion 130 of a second member (such as, but not limited to a reservoir housing 108), in a manner similar to the manner in which the first and second members 102 and 103 are connectable in the embodiment of FIGS. 1-6. More specifically, the housing 104 forms a receptacle (similar to the receptacle 110 described above for FIGS. 1-6) and contains a septum 116 that functions as a first septum (similar to the first septum 116 of FIGS. 1-6).

Similar to the embodiment of FIGS. 1-6, the connection portion 130 in FIG. 7 also includes a second septum 136. In particular, the connection portion 130 may be inserted into the receptacle formed by the housing 104, to connect the interior of the reservoir housing 108 in fluid-flow communication with the cannula 148. The cannula 148 in FIG. 7 may include a sharp end 148a adjacent the septum 116. As the connection portion 130 is inserted into the housing 104, the connection portion will push the septum 116 against the sharp end 148a of the cannula 148, to cause the sharp end 148a of the cannula 148 to pierce the septum 116. Further insertion motion of the connection portion 130 into the housing 104 causes the sharp end 148a of the cannula 148 to pierce the septum 136 in the connection portion 130, to form a flow path from or to the connection portion 130, through the cannula 148.

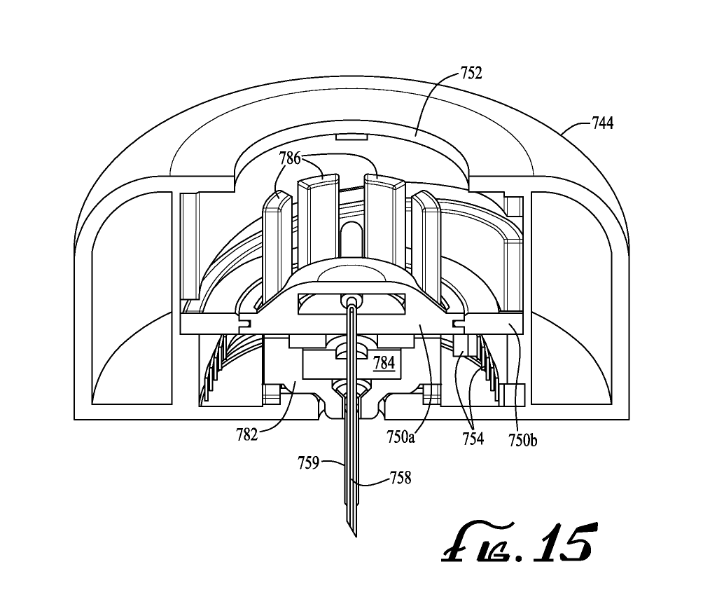

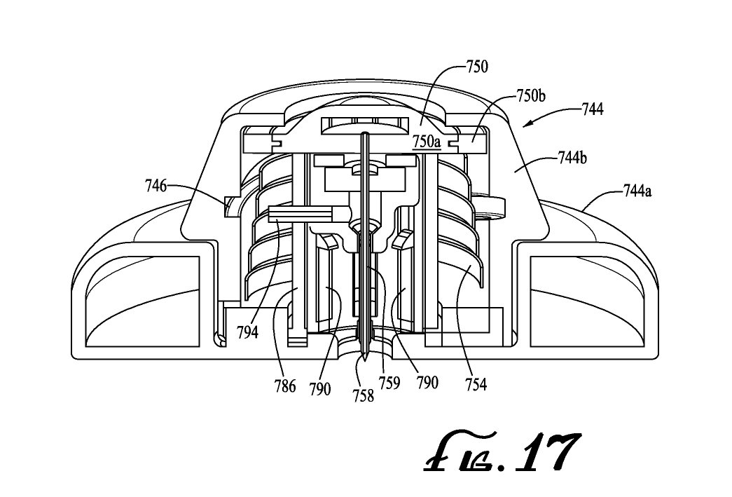

### Embodiment of FIGS. 11-13

A further embodiment of a structure for connecting a drive mechanism to a reservoir plunger is described with reference to FIGS. 11-13. In FIG. 11, a reservoir 200 has a housing 202 with a hollow interior for containing a fluidic medium, as described above. A plunger head 204 is located within the reservoir housing 202 and is moveable in the axial direction A of the reservoir, to expand or contract the interior volume of the reservoir. A pair of rods 206 and 207 extend from the plunger head 204, outside of the reservoir housing 202. The rods 206 and 207 function to provide a rigid connection between a U-shaped nut 208 and the plunger 204. The U-shaped nut 208 may be supported by the rods 206 and 207. Alternatively or in addition, the U-shaped nut 208 may be supported by a guide rail 210 for movement in the axial direction A of the reservoir 200.

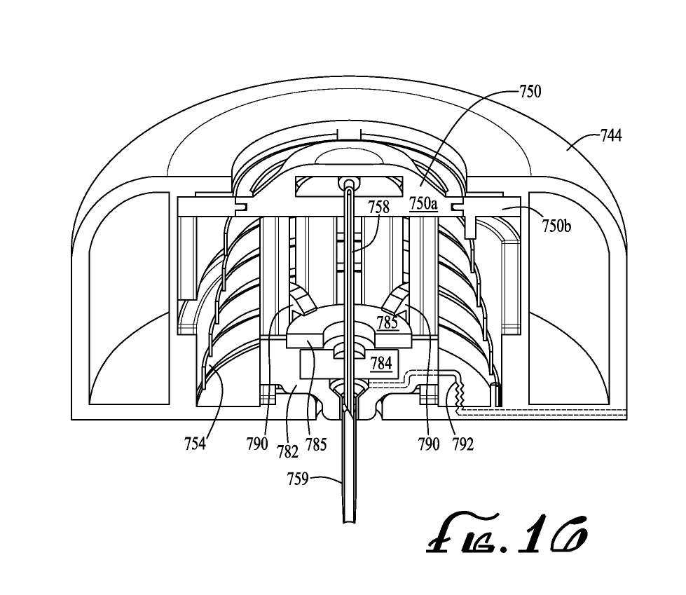

In FIG. 12, the U-shaped nut 208 has a pair of arms 208a and 208b that are connected by a span 208c and form a channel 210 there-between. In FIG. 11, he reservoir 200 is configured to be supported on the base 106, with the open side of the channel 210 of the U-shaped nut 208 oriented away from the base 106. A durable housing portion 212 is configured to secure to the base 106, over the reservoir 200. The durable housing portion 212 contains, among other components described above, a threaded drive shaft 214 that is operatively engaged with a drive device as described above. In FIG. 12, the drive shaft 214 is positioned within the durable housing portion 212 at a location at which it will fit within the channel 210 and engage the arms 208a and 208b, upon the durable housing portion 212 being arranged onto the base 106 for connection to the base 106. The channel 210 of the U-shaped nut 208 may have a sufficient depth to allow engagement of the drive shaft 214 with the arms 208a and 208b at any one of multiple locations of the drive shaft 214 in the dimension Z in FIG. 12, for ease of assembly and manufacturing tolerances. In particular embodiments, the placement of the durable housing portion 212 onto the base 106 in a position at which the durable housing portion 212 connects to the base 106 will also effect an alignment of the drive shaft 214 with the channel 210 of the U-shaped nut 208, so that no additional manipulation of the components are needed to operatively connect the drive shaft 214 to the nut 208.

In FIG. 12, the arms 208a and 208b of the U-shaped nut 208 may be offset in the axial direction A relative to each other and may be configured to engage threads on the drive shaft 214. As the drive shaft 214 is rotated while engaged with the U-shaped nut 208, the U-shaped nut 208 will be caused to move in the axial direction A. By abutting and/or connecting the U-shaped nut 208 against one or both of the rods 206 and 207, movement of the U-shaped nut 208 in the axial direction A is transferred to movement of the rods 206 and 207 and, thus, movement of the plunger head 204 in the axial direction A. Accordingly, when the drive shaft 214 is engaged with the U-shaped nut 208, movement of the reservoir plunger 204 may be selectively carried out and controlled by selectively driving the drive shaft 214.

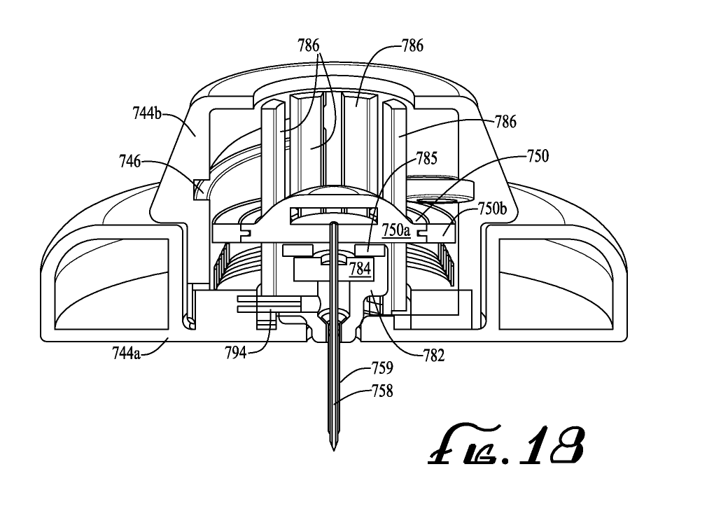

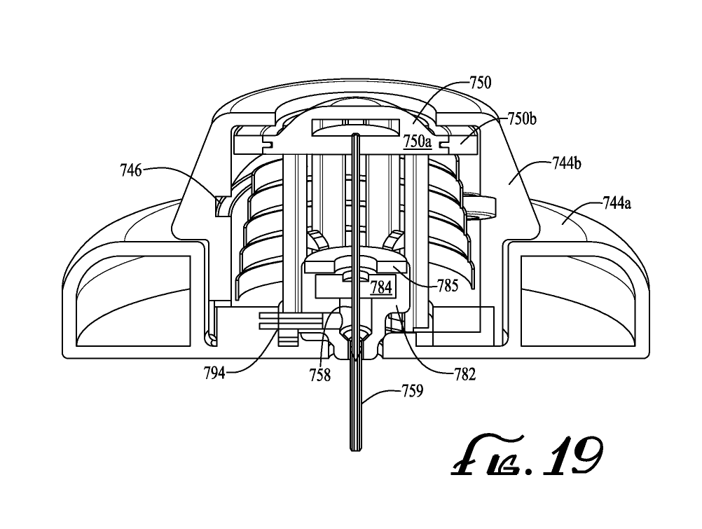

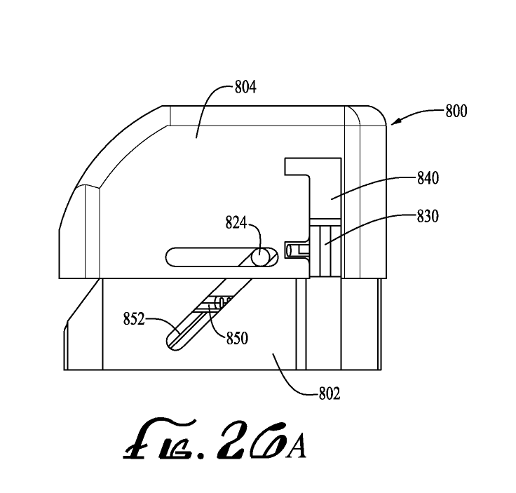

### Embodiment of FIGS. 14-21

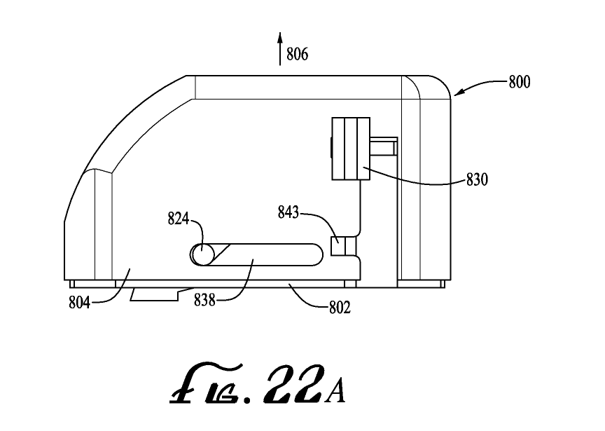

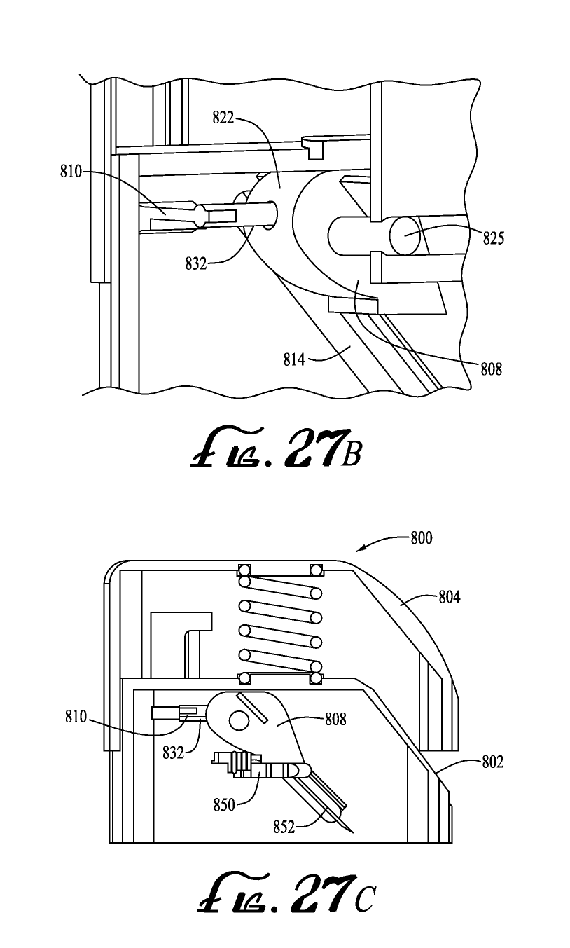

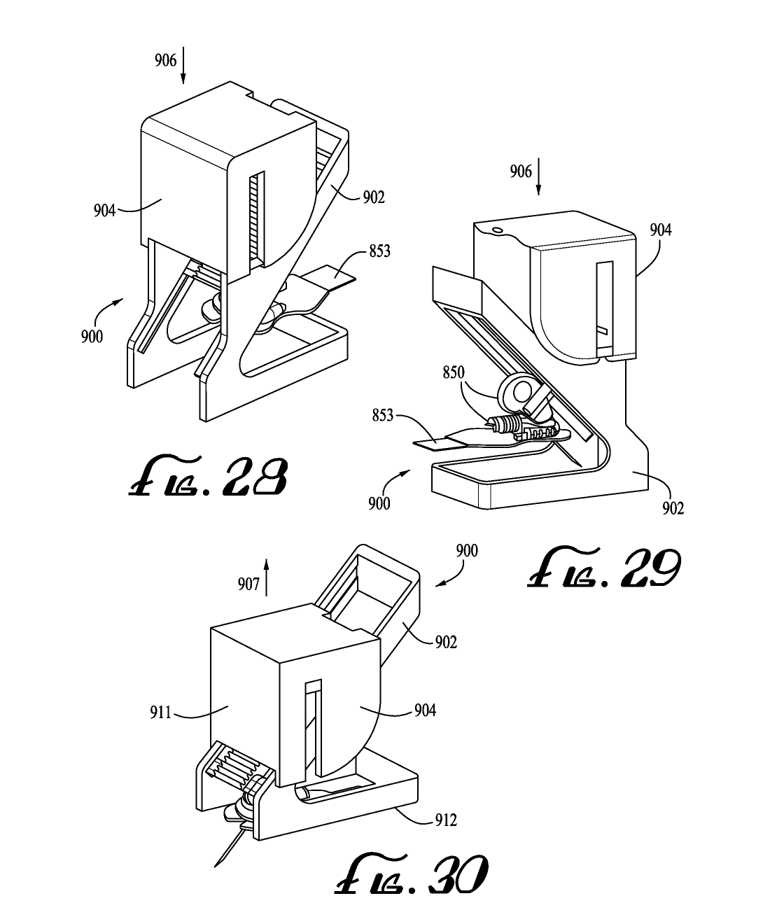

A further embodiment of a needle inserter device 712 is described with respect to FIGS. 24-25 in U.S. patent application Ser. No. 11/645,435, titled “Infusion Medium Delivery System Device And Method With Needle Inserter And Needle Inserter Device And Method” (assigned to the assignee of the present invention), which is incorporated herein by reference. Further aspects and variations of the needle inserter device 712 described in the above-referenced patent application are described herein with reference to FIGS. 14-21. Features and components of the structure shown in FIGS. 14-21 are identified by reference numbers that correspond to reference numbers used in the above-referenced U.S. patent application Ser. No. 11/645,435 for the same or similar features. A needle inserting device according to one embodiment of the invention is described with reference to FIGS. 14-16, while a needle inserting device according to a further embodiment of the invention is described with reference to FIGS. 17-21.

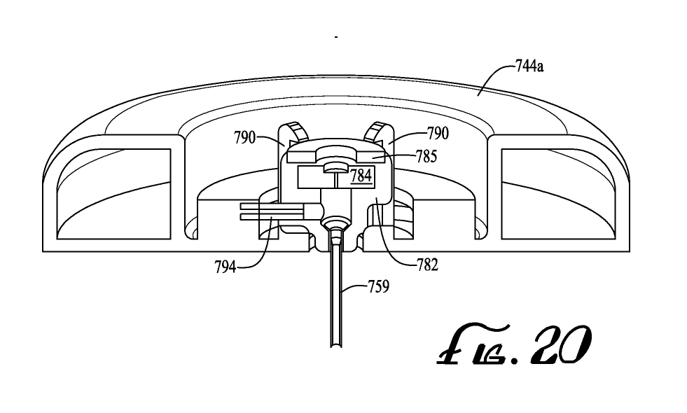

In FIG. 14, the needle inserter device 712 is in a starting position. In FIG. 15, the needle inserter device 712 is in an extended position. The needle inserter device 712 (shown in FIG. 14) includes a housing portion 744. The housing portion 744 may be part of or included within or connected to a further housing that contains other components of a system, such as, but not limited to, a reservoir, a drive device, linkage structure, and control electronics as described in the above-referenced U.S. patent application Ser. No. 11/645,435. In particular embodiments, the housing portion 744 may be part of or included within or connected to a disposable housing portion that connects to a durable housing portion as described in the above-referenced U.S. patent application Ser. No. 11/645,435.

In other embodiments, the needle inserter device 712 may be part of, located in or connected to the durable housing portion or an injection site module connected to the disposable housing portion or the durable housing portion, as described in the above-referenced U.S. patent application Ser. No. 11/645,435. Alternatively, the needle inserter device 712 may be included in other systems that operate by inserting a needle into a subject or object. The housing 744 may include a rigid, generally cylindrical or disc-shaped body, having a hollow, generally cylindrical interior and a longitudinal dimension along the axis Aₗ of the generally cylindrical shape of the body. The interior surface of the housing 744 has a spiral groove 746 that starts near, but spaced from, the top of the housing 744 (relative to the orientation shown in FIG. 14) and extends around the inner peripheral wall of the housing 744, to a location near the base of the housing 744. A further, linear groove (not shown in FIG. 14, but shown at 748 in FIG. 24 of the above-referenced U.S. patent application Ser. No. 11/645,435) is provided at the base end of the spiral groove and extends toward the top end of the housing (relative to the orientation shown in FIG. 14). The linear groove connects the base end of the spiral groove 746 with the top end of the spiral groove 746 and extends a short distance above the top end of the spiral groove 746.

A cam member 750 is located within the interior of the housing 744 and has a projecting outer peripheral edge 751 that extends into the grooves 746. The housing 744 includes an opening 752 on one end (the top end in the orientation of FIG. 14), through which the cam member 750 may be operated by manual or automated force. A surface of the cam member 750 may be exposed through the opening 752. That exposed surface of the cam member 750 may include a convex-shape, that extends into or partially through the opening 752, when the cam member 750 is in a retracted position, as shown in FIG. 14. The housing 744 also includes a needle opening 754 through the base of the housing 744, through which a needle and cannula may be extended, as described below.

The cam member 750 is supported within the interior of the housing 744 by a coiled torsion spring 754. The spring 754 extends between the cam member 750 and the base of the housing 744 and has one end secured to (or adjacent to) the base portion of the housing 744 and another end secured to the cam member 750.

In the starting or retracted position of FIG. 14, the coil spring 754 is partially unwound against its natural wound state, such that the spring 754 imparts a force on the cam member 750, in the winding direction of the spring. However, because the projecting edge 751 of the cam member 750 is located within a section of the linear groove that is offset from the upper end of the spiral groove 746 (as shown in FIG. 24 of the above-referenced U.S. patent application Ser. No. 11/645,435), the spring 754 is held in the partially unwound state, against the natural winding force of the spring 754.

From the retracted position shown in FIG. 14, a manual or automated force may be applied to the cam member 750, through the opening 752 in the housing 744 (such as a downward directed force relative to the orientation in FIG. 14), to force the cam member to move in the axial direction A₁, along the direction of arrow 755 and partially compress the coil spring against the natural compression force of the spring, until the cam edge 751 moves along the linear groove (groove 748 in the above-referenced U.S. patent application Ser. No. 11/645,435), toward the base of the housing 744 to align with the top end (relative to the orientation of the drawings) of the spiral groove 746. Once the cam edge 751 is aligned with the spiral groove 746, the natural winding force of the spring 754 causes the cam member 750 to rotate and move toward the base of the housing 744, while the cam edge 751 follows the spiral groove 746, as the spring winds toward its natural, non-tensioned state of winding. However, as the cam member 750 moves toward the base of the housing 744, the cam member 750 compresses the spring 754 against its natural longitudinal dimension (in the dimension from the of the axis A₁).

As the cam member 750 moves toward the base of the housing 744, a needle 758 is moved through the opening 754 in the base of the housing 744, to the extended position (shown in FIG. 15). The needle 758 is secured to a surface of the cam member that faces the base, so as to move with the cam member from the start or retracted position of the cam member 750 and needle 758 (shown in FIG. 14) to the extended position of the cam member 750 and needle 758 (shown in FIG. 15).

A cannula 759 may be supported on the shaft of the needle 758, adjacent the sharp end of the needle. One end of the cannula 759 may be flared or attached to a head portion 780 that is secured to a moveable carriage 782. The carriage 782 is located within the housing 744, between the moveable cam member 750 and the base and needle opening 754 of the housing 744. The carriage 782 is supported within the housing 744 for movement in the axial direction Aₗ with movement of the cam member 750 in the axial direction Aₗ.

The carriage 782 may include a body made of any suitably rigid material, such as, but not limited to plastic, metal, ceramic, composite material or the like. The body of the carriage 782 may include a central passage through which the needle 758 extends. A septum-like seal member 784 may be held within the body of the carriage 782. The needle 758 may extend through the seal member 784, and be slid through the seal member 784, while the seal member 784 forms a seal around the outer periphery of the needle 758. A retainer, such as, but not limited to, a generally rigid annular disk-shaped washer structure 785 may be arranged adjacent the seal member 784 to help retain the seal member 784 within the body of the carriage 782 and to provide additional rigidity to the seal member 784, while also providing a central passage through which the needle 758 may extend and move.

The carriage 782 has a surface 782a (the upper surface in the orientation shown in FIG. 14) that engages (or, at least, receives a force from) the cam member 750, as the cam member 750 is moved from the starting state of the cam member (shown FIG. 14) to the extended state of the cam member (shown in FIG. 15), to move the carriage 782 from its starting state (also shown in FIG. 14) to its extended state (also shown in FIG. 15). A guide structure 786 may be provided within the housing 744, for example, as an integral part of the housing 744 or, alternatively, as a separate structure that is secured to the base of the housing 744. The guide structure 786 may include one or more walls, rails or other suitable structure that engages one or more surfaces of the carriage 782 as the carriage is moved from its starting state (shown in FIG. 14) to its extended state (shown in FIG. 15). In one embodiment, as shown in FIG. 14, the guide structure 786 may include a tubular-shaped structure having a generally hollow cylindrical shape, with one or more slots or grooves extending in the axial dimension Aₗ along the cylindrical wall of the guide structure to receive a corresponding one or more projections 788 extending from the carriage 782. The projection(s) 788 ride along the axial slots or grooves in the generally cylindrical wall of the guide structure 786, as the carriage 782 is moved in the axial dimension A₁.

Once the carriage 782 is moved from its start state (shown in FIG. 14) to its extended state (FIG. 15), the carriage 782 may be arranged in a location at which one or more locking mechanisms operate to lock the carriage 782 in place in its extended state position. In the illustrated embodiment one or more locking mechanisms may be provided by one or more flexible pawls 790. The flexible pawls 790 may be formed as part of the guide structure 786 or may be adjacent the guide structure 786. Each pawl 790 includes a flexible arm portion that extends along the axial direction A₁, from the base of the housing 744 toward the opening 752. Each pawl 790 also includes a head 790a that has a stop surface for engaging the carriage structure 782, to inhibit further movement of the carriage structure 782 in the axial direction A₁, once the carriage structure 782 has been moved to its extended state (shown in FIG. 15). In the illustrated embodiment, the pawls 790 are arranged to engage either or both the surface 782a of the carriage 782 or the retainer 785, when the carriage 782 is in the extended state (shown in FIG. 15). Each pawl 790 may have an angled surface 790b, for engaging the carriage 782 as the carriage is moved from its start state (FIG. 14) to its extended state (FIG. 15) and allow the carriage to push and flex the pawls radially outward (relative to the axis A₁) sufficient to allow the carriage 782 to pass the pawl heads 790 during the motion of the carriage toward its extended state.

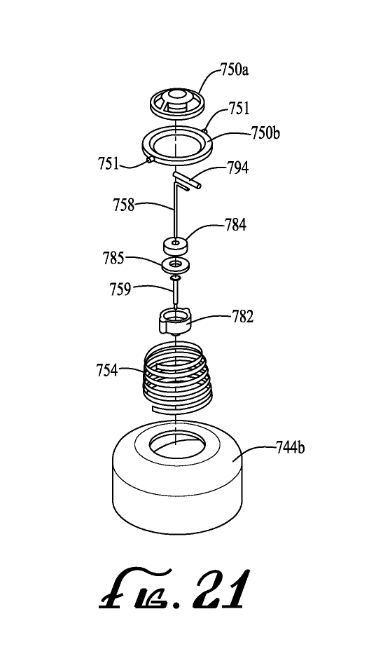

Once the carriage 782 has been moved to its extended state (by the action of the movement of the cam member 750 to its extended state), the carriage 782 may be locked in place relative to the housing 744, by the pawls 790. Then, cam member 750 may be acted upon by the compression force of the spring 754 and may follow the linear groove (groove 748 in the above-referenced U.S. patent application Ser. No. 11/645,435) to move to its retracted state (shown in FIG. 16). As the cam member 750 moves to its retracted state, the cam member 750 moves the needle 758 in the axial direction A₁, to at least partially withdraw the needle 758 from the cannula 759 to open a fluid flow path into the cannula 759, through the cannula head 780.

A fluid flow path to or from the cannula head 780 may be provided through the body of the carriage 782, and through a flexible conduit 792 attached to the carriage 782, as shown in FIG. 16. The conduit 792 may have sufficient flexibility and/or slack to allow the carriage 782 to move between its start state (shown in FIG. 14) and its extended state (shown in FIG. 15), while the conduit 792 remains attached to the carriage 782. The conduit 792 may extend and provide fluid flow communication to or from one or more of a reservoir, sensor structure, or other suitable fluid containing or processing mechanism (not shown in FIGS. 14-16).

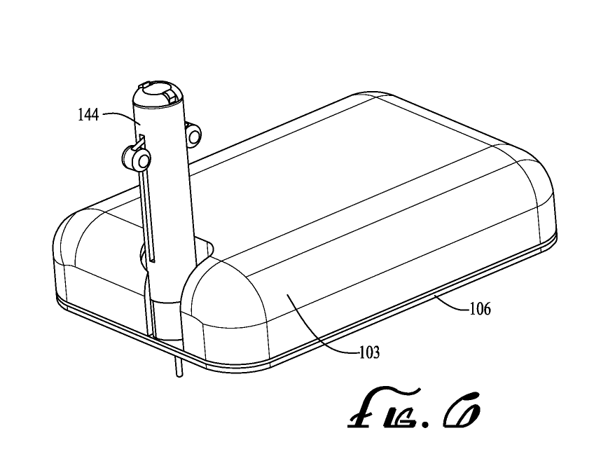

Alternatively, the fluid flow passage through the body of the carriage 782 (shown in broken lines in FIG. 16) may be arranged to automatically align with a fluid flow path or conduit supported in the housing 744, when the carriage 782 reaches its extended state (shown in FIG. 15), to complete a fluid flow path to or from one or more of a reservoir, sensor structure, or other suitable fluid containing or processing mechanism (not shown in FIGS. 14-16). In yet further embodiments, the carriage 782 and the housing 744 may be provided with a needle and septum structure (similar to the needle 50 or 150 and septum 54 or 154 described in connection with the embodiments of FIGS. 4-8 of the above-referenced U.S. patent application Ser. No. 11/645,435), which has been incorporated herein by reference, in its entirety, for connecting the cannula 759 in fluid-flow communication with a reservoir, sensor structure or other fluid containing or processing mechanism.

Thus, by supporting the base of the housing 744 at an injection site, the housing 744 may be arranged adjacent a patient-user's skin to allow the sharp end of the needle 758 to pierce the patient-user's skin and to allow the cannula around the needle shaft to be inserted at least partially into the patient-user's skin, when the needle is in the extended position of FIG. 15.

In the extended position (FIG. 15), the carriage 782 is locked in place, relative to the housing 744. Also, once the needle 758 and cannula 759 are in the extended position of FIG. 15, the cam projection 751 (which had followed the spiral path of the groove 746) is aligned with the linear groove (groove 748 in the above-referenced U.S. patent application Ser. No. 11/645,435). At that position, the spring 756 is extended in the longitudinal dimension of axis A₁ beyond its natural longitudinal state. Accordingly, the spring 756 provides a force on the cam member 750, to move the cam member 750 in the axial dimension A₁, in the direction opposite to the direction of arrow 755, while the projection 751 follows the linear groove (groove 748 in the above-referenced U.S. patent application Ser. No. 11/645,435), to the retracted position of FIG. 16.

As the cam member 750 is moved, under the compression force of the spring 754, to the retracted state, the needle 758 at least partially withdraws from the cannula 759 and opens a fluid flow path from the conduit 792 to the cannula 759, through a passage in the body of the carriage 782. Accordingly, the cannula may be inserted into a patient-user's skin and connected in fluid flow communication with the conduit 792 (and with a reservoir, sensor structure or other fluid containing or processing mechanism that is also connected in fluid flow communication with the conduit 792).

As described above, during movement of the cam member 750 in the axial direction A₁, from its start state (shown in FIG. 14) to its extended state (shown in FIG. 15), the cam member 750 is acted upon by the unwinding force of the spring 754 and follows a spiral groove 746 in the interior wall of the housing 744. As a result, the cam member 750 rotates around the axis A₁, during its movement from the start state to the extended state.

In particular embodiments, the cam member 750 may include an outer circumference portion 750a and an inner portion 750b, where the outer circumference portion 750a is connected to, but allowed to rotate (about the axis A₁) relative to the inner portion 750b of the cam member 750. A section of the spring 754 may be secured to the outer portion 750a of the cam member, such that an unwinding movement of the spring 754 will cause a rotational motion of the outer portion 750a of the cam member.

The outer portion 750a of the cam member may be connected to the inner portion 750b of the cam member by a tab and groove configuration, wherein one of the outer or inner portions 750a or 750b (the outer portion 750a in the illustrated embodiment) is provided with an annular tab that extends toward the other of the outer or inner portions 750a and 750b. The other of the outer and inner portions 750a and 750b (the inner portion 750b in the illustrated embodiment) is provided with an annular groove that aligns with and receives the annular tab. The annular tab and groove arrangement allows the outer and inner portions 750a and 750b of the cam member 750 to move together in the axial direction A₁, yet allows that outer portion 750a to rotate relative to the inner portion 750b around the axis Aₗ. Accordingly, the outer portion 750a of the cam member 750 may rotate under the unwinding action of the spring 754 and the direction of the spiral groove 746 as the cam member 750 moves in the axial direction Aₗ from its start state (FIG. 14) to its extended state (FIG. 15). However, during such motion, the inner portion 750b of the cam member 750 need not rotate with the outer portion 750a. As a result, the needle 758 need not rotate about the axis Aₗ as the cam member 750 moves from its start state to its extended state. In some contexts, user-patient comfort may be improved by inhibiting rotation of the needle 758, as the needle 758 and cannula 759 are inserted into the patient-user's skin.

In particular embodiments, the inner portion 750b of the cam member 750 may be held from rotating about the axis A₁ by retaining structure. For example, the inner portion 750b may engage one or more surfaces of the guide structure 786 as the cam member 750 moves in the axial direction A₁, to inhibit rotation of the inner portion 750b about the axis A₁. In the illustrated embodiment, the inner portion 750b of the cam member includes one or more slots or openings through which leg portions of the guide structure 786 extend. The engagement of the inner portion 750b with the one or more leg portions of the guide structure 786 inhibit rotation of the inner portion 750b about the axis A₁. In other embodiments, other suitable structural configurations may be employed to inhibit rotation of the inner portion 750b of the cam member 750 about the axis A₁.

In the embodiment in FIGS. 14-16, the needle 758 of the needle injecting structure remains in the housing 744 with the cannula 759, after the cannula has been inserted into the patient-user's skin and the needle 758 has been moved to its retracted position (shown in FIG. 16). In other embodiments, the needle injecting structure may be composed of multiple, separable parts that may be separated after the cannula has been moved into its extended state (and inserted into the patient-user), for removing the needle 758 (and other structure associated with the needle 758) from a base portion that holds the cannula in its extended state. An example of a multi-piece structure is shown in FIGS. 17-21. The structure and function of the embodiment in FIGS. 17-21 is similar to that of the embodiment described above for FIGS. 14-16, except that the housing 744 in FIGS. 17-21 has two parts including a base portion 744a and a nest portion 744b that is removable from the base portion 744a. Accordingly, corresponding reference numbers are used for corresponding components and reference is made to the above description of corresponding structure and function.

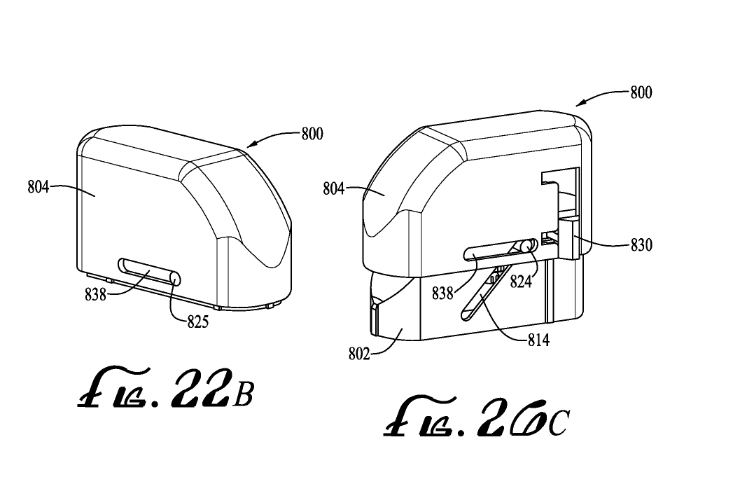

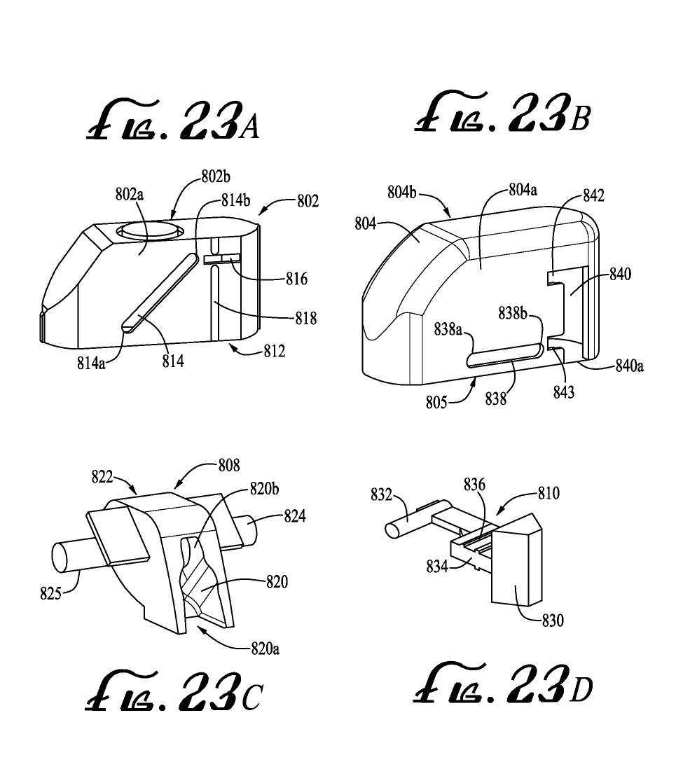

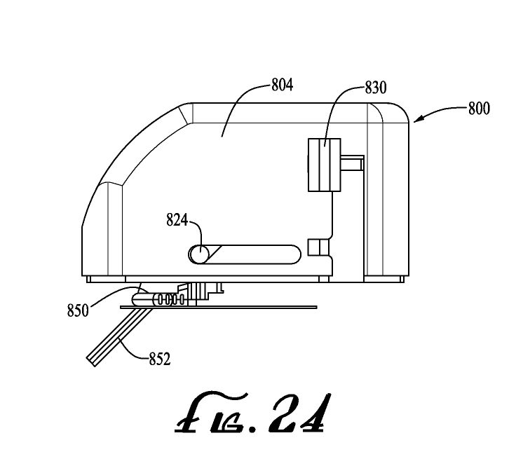

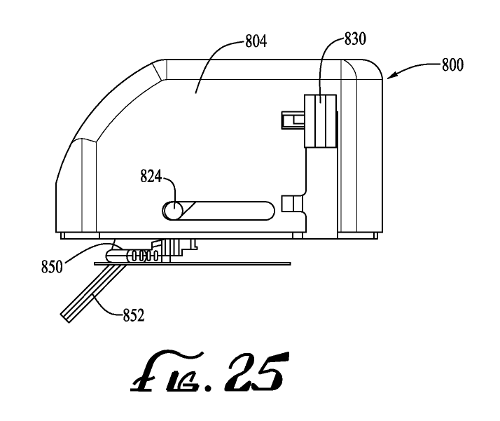

In FIG. 17, the multi-piece needle inserting device is shown in the start state, corresponding to the start state of the above-described embodiment shown in FIG. 14. In FIG. 18, the multi-piece needle inserting device is shown in the extended state, corresponding to the extended state of the above-described embodiment shown in FIG. 15. In FIG. 19, the multi-piece needle inserting device is shown in the retracted state, corresponding to the retracted state of the above-described embodiment shown in FIG. 16. In FIG. 20, various components of the example multi-piece needle inserting device are shown, in an exploded view.

In the embodiment of FIGS. 17-21, a fluid flow connection is provided to or from the cannula 759, through a tubing structure 794 that is extends through and/or is connected in fluid flow communication with a fluid passage through the body of the carriage 782. When the carriage 782 is moved to its extended state (shown in FIG. 18), the tubing structure 794 aligns with a fluid-flow passage formed in (or otherwise provided in) the base portion 744a of the housing 744. In particular embodiments, the tubing structure 794 may include a resiliently flexible tubing (made of a flexible material, such as, but not limited to, silicon, plastic, rubber or the like) that allows the tubing to bend and pass over a portion of the base structure as the carriage 782 moves to its extended state and then resiliently flex back to its natural shape to extend into an opening of a fluid flow passage in the base portion 744a of the housing 744 (as shown in FIGS. 18 and 19).

After the cam member 750 has moved to its retracted state (FIG. 19), the nest portion 744b of the housing 740 may be removed from the base portion 740a of the housing, as shown in FIG. 20. As a result, the base portion 744b of the housing may remain on the patient-user's skin, with the cannula 759 inserted into the patient-user, while the needle 758 (and other components, such as the spring 754 and cam member 750) may be removed by removing the nest portion 744a of the housing 744 from the base portion 744b. The base portion 744b may be integral with or connected to a disposable housing portion, a durable housing portion, a base of a disposable housing portion, a base of a durable housing portion or a separate injection site housing structure that may be connected to a durable housing portion, a disposable housing portion or the like. Examples of such various arrangements of needle inserting devices are described in the above-referenced U.S. patent application Ser. No. 11/645,435), which has been incorporated herein by reference, in its entirety.

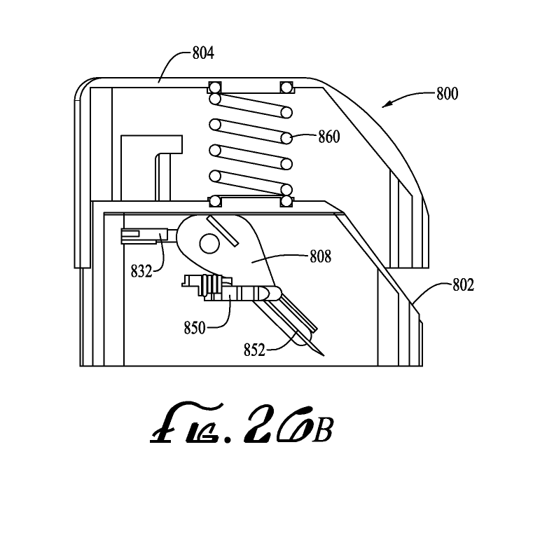

### Embodiments of FIGS. 22-27

FIGS. 22-27 illustrate an example embodiment of a needle inserting device 800 for inserting a needle and cannula or a hollow needle into a patient-user (or other subject) for fluid-flow connection to a further device, where the needle and/or cannula are inserted at an angle (a non-perpendicular angle relative to the patient-user's skin), such as, but not limited to, an angle within the range of 20° and 60° and, in particular embodiments, about 45° relative to the patient-user's skin (or insertion surface of another subject). In the illustrated embodiment, the further device is a sensor device, wherein insertion of a hollow needle or cannula into a patient-user (or other subject) provides a fluid flow connection between sensing material or electronics in the sensor device and the patient-user (or other subject). However, embodiments of the invention may be used for inserting a needle associated with other devices that require the insertion of a needle into a patient-user (or other subject), such as, but not limited to an infusion medium delivery device that has a reservoir for containing an infusion medium, wherein insertion of a hollow needle or cannula into a patient-user (or other subject) provides a fluid-flow connection between the reservoir and the patient-user (or other subject).

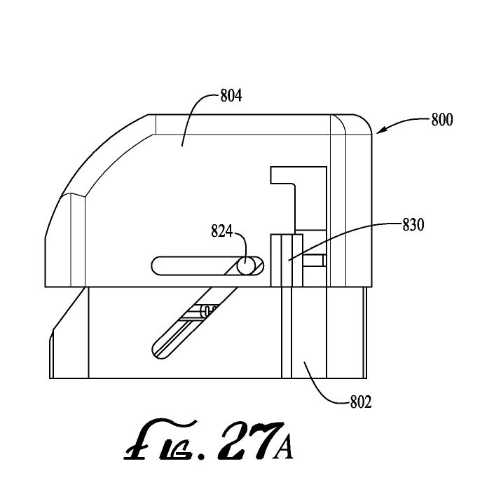

In FIGS. 22a and 22b, the needle inserting device 800 is shown in an assembled state, in initial position. In FIGS. 23a-23b, components of the needle inserting device 800 are shown, separate from each other. The needle inserting device 800 includes a base structure 802 (FIG. 23a), a cap structure 804 (FIG. 23b) that fits over the base structure (as shown in FIGS. 22a and 22b) and is moveable in a sliding motion in the direction of arrow 806 relative to the base structure 802. The needle inserting device 800 also includes a slide structure 808 (FIG. 23c) and an extractor structure 810 (FIG. 23d), each of which are located within the base structure 802 and moveable relative to the base structure 802. Each of the components 802, 803, 808 and 810 may be made of any suitably rigid material such as, but not limited to plastic, metal, ceramic, composite material, or the like. In particular embodiments, those components may be made of molded plastic material, for manufacturing efficiency and ease.

As shown in FIG. 23a, the base structure 802 has a generally rigid body with a hollow interior for containing the slide structure 808 and the extractor 810. The body of the base structure 802 has a pair of generally parallel walls 802a and 802b. The body of the base structure 802 also has a bottom surface 812 that is configured to be arranged adjacent a patient-user's skin (or surface of another subject) during a needle injection operation of the device 800.

The body of the base structure 802 has an angled slot 814 in each of the parallel walls 802a and 802b (where the wall 802b is facing into the page of FIG. 23a and, thus, hidden from view in that drawing). Each slot 814 has a longitudinal dimension extending between first and second ends 814a and 814b of the slot 814, where the first end 814a of the slot is closer to the bottom surface 812 of the base structure than the second end 814b of the slot. Accordingly, in operation, the first end 814a of the slot is closer to the patient-user's skin (or surface of other subject) than the second end 814b of the slot.

One of the walls 802a of the body of the base structure 802 has a second slot 816 that has a longitudinal dimension that is generally parallel to the bottom surface 812 of the base structure 802. The slot 816 is located adjacent the second end 814b of the slot 814. One or both of the walls 802a of the body of the base structure 802 has a groove (or a further slot) 818 that has a longitudinal dimension that is generally perpendicular to the bottom surface 812 of the base structure 802. Accordingly, in operation, the longitudinal dimension of the groove (or further slot) 818 is generally perpendicular to the patient-user's skin or surface of other subject to be injected).

The slide structure 808 (FIG. 23c) has a generally rigid body that forms a receptacle 820 for receiving and holding a device having a cannula (or hollow needle) assembly during operation. The device having a cannula (or hollow needle) assembly may be a sensor device, a needle set for connection to an infusion device or other device, or the like. The receptacle 820 in the illustrated embodiment includes a cup-shaped recess that is open on one side 820a and has a second side 820b that is open to a channel through the body of the slide structure 808. The channel (hidden from view in FIG. 23c) is also open on the rear side 822 (relative to the orientation shown in FIG. 23c) of the body of the slide structure 808. In other embodiments, the receptacle 820 may have any suitable configuration that is capable of holding and selectively releasing a device having a cannula (or hollow needle) assembly.

A pair of shafts or arms 824 and 825 protrude and extend from opposite sides of the body of the slide structure 808, generally perpendicular to the above-described channel through the body of the slide structure 808. When assembled with the base structure 802 (as shown in FIGS. 22a and 22b), the slide structure 808 is arranged inside the hollow interior of the base structure 802, with the arms 824 and 825 extended through the slots 814 in the sides 802a and 802b, respectively, of the base structure 802. The slide structure 808 is moveable within the interior of the base structure 802, as the arms 824 and 825 slide within the slots 814 in the respective sides 802a and 802b of the base structure. Accordingly, the angled direction of the slots 814 guide the motion of the slide structure 808 in an angled direction relative to the bottom surface 812 of the base structure 802 (and to the patient-user's skin or surface of other subject to be injected).

The extractor structure 810 (FIG. 23d) has a handle portion 830 that is located external to the base structure 802, when the extractor structure 810 is assembled inside of the interior of the base structure (as shown in FIG. 22a). The extractor structure 810 also has a shaft portion 832 that is configured to fit within at least a portion of the channel through the body of the slide structure 808, through the opening in the side 822 of the body of the slide structure 808. As described below, selective movement of the shaft portion 832 into the channel of the slide structure 808 may be carried out by manual operation of the handle portion 830, to selectively push a device having a cannula or hollow needle out of or in another release position relative to the receptacle 820 in the slide structure 808.

The extractor structure 810 has a connection portion 834, connecting the handle portion 830 to the shaft portion 832. The connection portion 834 is configured to extend through the slot 816 in the body of the base structure 802 and is moveable in the longitudinal direction of the slot, when the extractor structure 810 is assembled inside of the interior of the base structure (as shown in FIG. 22a). The connection portion 834 may be provided with a guide 836 for stabilizing and smoothing the motion of the extractor structure 810. The guide 836 may include one or more surfaces having a channel (formed between a pair of ribs in the illustrated embodiment) arranged generally parallel to the longitudinal dimension of the slot 816 (when the extractor structure 810 is assembled with the base structure 802). The channel has a width dimension that is greater than the thickness dimension of the wall 802a of the base structure 802, to allow the channel in the guide 836 to receive a portion of the wall 802a, when the extractor structure 810 is assembled with the base structure 802.

The cap structure 804 (FIG. 23b) has a generally rigid body that may be shaped similar to the shape of the body of the base structure 802, but slightly larger than the body of the base structure 802. The body of the cap structure 804 has a hollow interior and an open bottom side 805 (relative to the orientation shown in FIG. 23b), for receiving the base structure 802 when assembled in the manner shown in FIGS. 22a and 22b. The body of the cap structure 804 has a pair of generally parallel walls 804a and 804b, corresponding to the walls 802a and 802b, respectively, of the base structure 802.

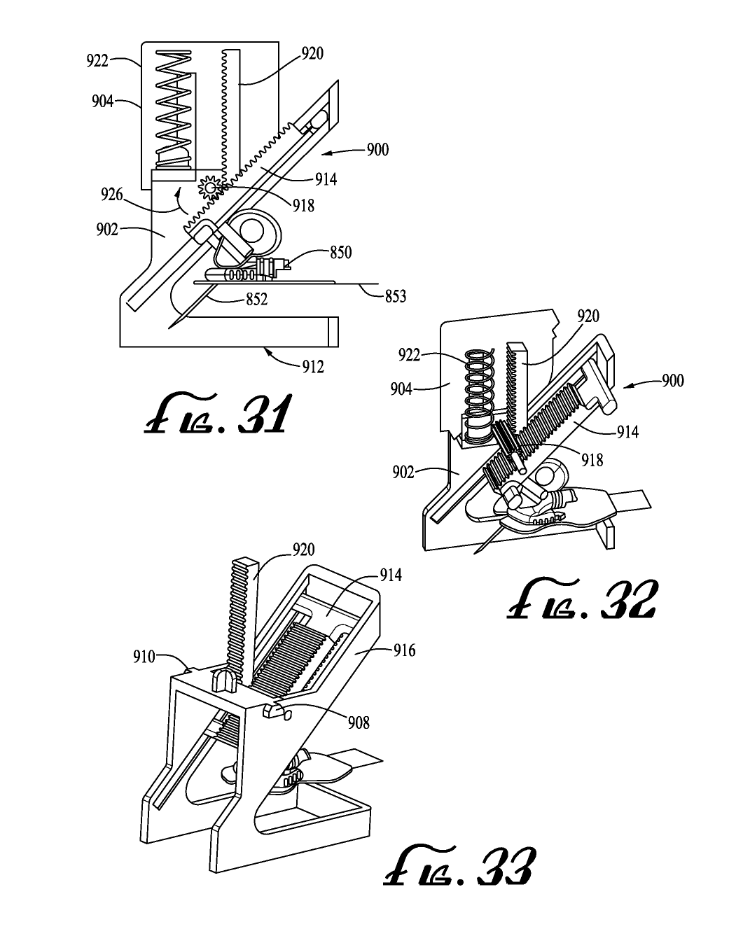

One or more ribs or other projections (not in view in the drawings) may be provided on the interior-facing surface of one or both of the walls 804a and 804b in a location to align with and fit within the groove (or slot) 818 in one or both of the walls 802a and 802b, respectively, of the base structure 802, when the cap structure 804 and the base structure 802 are assembled as shown in FIGS. 22a and 22b. When the cap structure 804 is assembled with the base structure 802, the cap structure 804 is moveable in the direction of arrow 806 from an initial position (FIG. 22a) to a retracted position (FIG. 26a), and then in the direction opposite to the arrow 806 to an insertion position. The ribs or other projections on one or both of the walls 804a and 804b of the cap structure 804 ride along the groove (or slot) 818 on one or both of the walls 802a and 802b of the base structure 802 as the cap structure 804 is moved relative to the base structure 802 in the direction of (or opposite to) the arrow 806.

The body of the cap structure 804 has a slot 838 in each of the parallel walls 804a and 804b. Each slot 838 has a longitudinal dimension extending between first and second ends 838a and 838b of the slot 838, where the longitudinal dimension is generally parallel to the bottom surface 812 of the base structure 802 (when the cap structure 804 and the base structure 802 are assembled together) and, thus, during operation, generally parallel to the to the patient-user's skin or surface of other subject to be injected.

One of the walls 804a of the body of the cap structure 804 has a second slot 840 that has a longitudinal dimension that is generally perpendicular to the bottom surface 812 of the base structure 802 (when the cap structure 804 and the base structure 802 are assembled together). The slot 840 has a first end 840a that is open at the open bottom side 805 of the cap structure 804. The slot 840 has a second end 840b that is located at a distance from the open bottom side 805 corresponding to the longitudinal length of the slot 840. A first extension slot 842 extends laterally to one side of the slot 840, at the end 840a of the slot 840. The first extension slot 842 has a longitudinal dimension that is generally perpendicular to the longitudinal dimension of the slot 840. A second extension slot 843 extends laterally to one side of the slot 840, adjacent, but spaced from the open first end 840a of the slot 840. The second extension slot 843 also has a longitudinal dimension that is generally perpendicular to the longitudinal dimension of the slot 840. When the cap structure 804 is assembled with the base structure 802, slide structure 808 and extractor structure 810, the arms 824 and 825 of the slide structure 808 extend through the slots 838 in the body of the cap structure 804, and the connection portion 834 of the extractor structure 810 extends through the slot 840 and/or one of the extension slots 842 and 843 in the body of the cap structure 804, as shown in FIGS. 22a and 22b.

In operation, the needle inserting device 800 may come pre-assembled or may be assembled as shown in FIGS. 22a and 22b, with the slide structure and the extractor structure set in an initial position. In the initial position shown in FIGS. 22a and 22b, the cap structure 804 is arranged over the base structure 802 and is moved relative to the base structure 802 to the end of its full range of motion in the direction opposite to the direction of arrow 806. In the initial position, the bottom side 805 of the cap structure 804 is arranged adjacent to the bottom side 812 of the base structure 802. Also, in the initial position, the slide structure 808 is located such that the arms 824 and 825 are adjacent the end 814a of the slot 814 in the base structure 802 and adjacent the end 838a of the slot 838 in the cap structure 804.

Further, in the initial position of FIG. 22a, the extractor structure 810 is located in the first extension slot 842. The initial position of the extractor structure 810 inhibits relative movement between the cap structure 804 and the base structure 802 in the direction of arrow 806. The needle inserting device 800 may be shipped or stored in the initial position. Alternatively, the patient-user (or medical practitioner) may set the needle inserting device in the initial position, after retrieval from storage or shipping. In the initial position, the needle inserting device 800 may receive a device having a cannula or hollow needle for insertion into a patient-user (or other subject).

From the initial position of FIG. 22a, a patient-user (or a medical practitioner) may place a device having a cannula or hollow needle in the receptacle 820 of the slide structure 808, to place the needle inserter device in a loaded position. In FIG. 24, a needle inserter device 800 is shown with a sensor device 850 (having a needle and cannula structure 852) received within the receptacle of the slide structure 808, such that the needle inserter device 800 is in a sensor loaded position. In the loaded position, the needle and cannula structure 852 is arranged at an angle (a non-perpendicular angle) relative to the bottom surface 812 of the base structure 802 (and, thus, relative to the patient-user's skin or surface of subject to be injected, during an injection operation).

In further embodiments, the needle inserter device 800 may be shipped and/or stored in a loaded position, with a device (such as a sensor device 850) pre-loaded in the receptacle 820 of the slide structure 808, as shown in FIG. 24. In such pre-loaded embodiments, a removable cover (for removal prior to use of the device) may be provided over at least the portion of the device 800 holding the device 850, to protect the device 850 from damage and to inhibit accidental puncture from the sharp end of a needle or cannula extending from the device 850.

In the loaded position, the device 850 may be releasably locked in the receptacle 820 by any suitable releasable locking mechanism, including, but not limited to, a friction fit, a spring tab or the like. The locking mechanism may be configured to lock the device 850 in place and inhibit separation of the device from the receptacle 820 when the device 800 is placed in a loaded state, yet release the lock and allow the device 850 to be separated from the receptacle 820, by a releasing action of the extractor 810, as described below.

From the loaded position of FIG. 24, a patient-user (or medical practitioner) may set the extractor structure 810 of the device 800 into an unlock position shown in FIG. 25. The device 800 may be set to the unlock position by manually moving the handle portion 830 of the extractor structure 810 in the direction toward the slot 840, to align the connection portion 834 of the extractor structure 810 with the slot 840, as shown in FIG. 25.

From the unlock position of FIG. 25, the patient-user (or medical practitioner) may set the device 800 into a retracted position as shown in FIGS. 26a and 26c, and in a cut-away view in 26b. The device 800 may be set to the retracted position by moving the cap structure 804 relative to the base structure 802, in the direction of arrow 806, to the position shown in FIGS. 26a, 26b and 26c. Movement of the cap structure 804 relative to the base structure 802 may be carried out manually, by gripping the cap structure 804 and/or the base structure 802 and drawing the two structures partially apart.

Alternatively or in addition, a bias mechanism, such as, but not limited to a coil spring or other spring structure, magnets or the like, may be provided within the device 800, to bias the cap structure 804 and base structure 802 toward the retracted position shown in FIGS. 26b. For example, a coil spring 860 may be arranged between the cap structure 804 and the base structure 802, with one end of the coil coupled to the inside surface 862 of the upper wall of the cap structure 804 and the other end of the coil coupled to the outside surface 864 of the upper wall of the base structure 802 (relative to the orientation shown in FIG. 26b). The coil spring 860 may be configured to be in a compressed state (compressed against its natural length dimension) when the cap structure 804 and base structure 802 are in the initial, loaded and unlock positions of FIGS. 22a, 24 and 25, respectively, to impart a bias force directed toward separating the surface 862 of the cap structure 804 and surface 864 of the base structure 802. In other embodiments, a first magnet (such as a permanent magnet) may be arranged on or in the upper wall of the base structure 802 and a second magnet (such as a permanent magnet) may be arranged on or in the upper wall of the cap structure 804, with common poles of the two magnets facing each other to provide an opposing force directed toward separating the surface 862 of the cap structure 804 and surface 864 of the base structure 802.

By moving the base structure 802 and cap structure 804 to the retracted position (FIGS. 26a, 26b and 26c), the engagement of the arms 824 and 825 with the slots 838 in the side walls 804a and 804b of the cap structure 804 cause the slide structure 808 to move relative to the base structure 802 further into the interior of the base structure. As the slide structure 808 is moved further into the interior of the base structure, the arms 824 and 825 are guided by the angled slots 814 in the side walls 802a and 802b of the base structure 802, toward the second end 814b of the slots 814. By moving the slide structure 808 further into the interior of the base structure 802, the device 850 (including the needle or cannula portion 852 of the device 850) that is received in the receptacle 820 of the slide structure 808 is also drawn into the interior of the base structure 802.

In the retracted position, the device 800 may be arranged relative to a patient-user's skin (or surface of other subject to be injected) for injection of the needle or cannula portion 852 of the device 850. In particular, the bottom surface 812 of the base structure 802 may be arranged adjacent and generally parallel to the patient-user's skin (or surface of other subject to be injected) at a desired injection site.

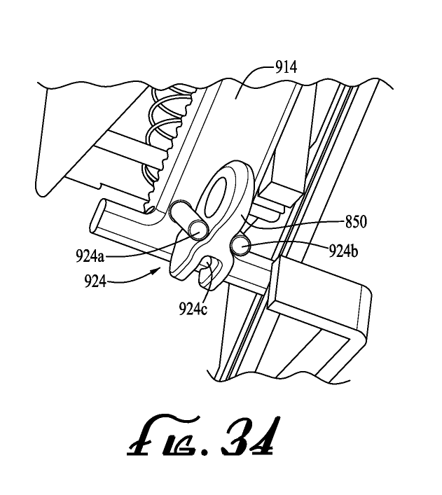

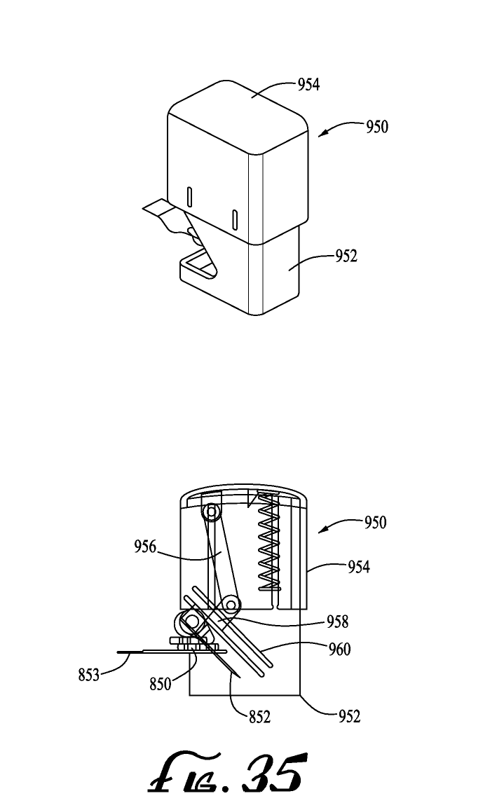

In the retracted position (FIGS. 26a, 26b and 26c), the extractor structure 810 aligns with the second extension slot 843. From the retracted position of FIGS. 26a, 26b and 26c, the device may be set to the needle extract position shown in FIGS. 27a, 27b and 27c, by moving the handle 830 of the extractor structure 810 into the extension slot 843. As the handle 830 of the extractor structure 810 is moved into the extension slot 843, the shaft portion 832 of the extractor structure 810 is moved into (or further into) the channel in the body of the slide structure 808, through the opening in the surface 822 of the body of the slide structure 808, to release the device 850 from being locked within the. For example, the movement of the shaft portion 832 into (or further into) the channel in the body of the slide structure 808 may cause the free end of the shaft portion 832 to contact the device 850 and physically push the device 850 out of a friction fit with the receptacle 820. Alternatively, or in addition, such movement of the shaft portion 832 may cause the shaft portion 832 to engage and move a flexible tab, spring or other lock mechanism out of locking engagement with the device 850.

Once the device 800 is set in the needle extract position (FIGS. 27a, 27b and 27c), the device 800 may be operated to insert the needle or cannula 852 of the device 850 into the patient-user (or other subject). While the device 800 may have already be arranged relative to a patient-user's skin (or surface of other subject to be injected) for injection when the device was set in the retracted position, as described above, in other embodiments, the device 800 may not be arranged relative to the patient user's skin (or surface of other subject to be injected) until after the device 800 is set in the needle extract position.

The device 800 is operated to insert the needle or cannula 852 at an angle (a non-perpendicular angle) relative to the patient-user's skin (or surface of other subject to be injected). To insert the needle or cannula 852 into the patient-user's skin (or surface or other subject), a force in the direction opposite to the direction of arrow 806 is applied to move the cap structure 804 relative to the base structure 802, from the needle extract position (FIG. 27a) toward an insert position. The force is sufficient to overcome the bias mechanism 860, to move the cap structure 804 over the base structure 802 to a position similar to the relative positions of the cap structure 804 and base structure 802 shown in FIG. 25. The force may be applied manually, for example, by the patient-user (or medical technician) pressing downward (in the orientation of FIG. 27a) on the cap structure 804 at a desired velocity and timing. Alternatively, the force may be applied by an automated device, in response to an activation signal.

With the relative motion of the cap structure 804 and the base structure 802 from the needle extract position (FIG. 27a) toward the insert position, the arms 824 and 825 of the slide structure 808 are engaged by the slots 838 in the side surfaces 804a and 804b of the cap structure 804 and are moved downward (relative to the orientation of FIG. 27a). As the arms 824 and 825 move downward (relative to the orientation of FIG. 27a), the arms 824 and 825 are guided by the angled slots 814 in the base structure 802, to move the needle or cannula 852 at an angle relative to the bottom surface 812 of the base structure 802 (and, thus, at an angle relative to the patient-user's skin or the surface of other subject to be injected). The angled orientation of the needle or cannula 852 and the angled insertion direction provided by the angled slots 814, result in an insertion of the needle or cannula 852 at an angle (a non-perpendicular angle) relative to the patient-user's skin (or surface of other subject to be injected).

Accordingly, with the device 800, a force in a direction opposite to the arrow 806 and generally perpendicular to the patient-user's skin (or surface of other subject to be injected) results in an insertion of a needle or cannula 852 at an angle (a non-perpendicular angle) to the patient-user's skin (or surface of other subject). The angle of the slots 814 relative to the bottom surface 812 of the base structure 802 define the angle of insertion of the needle or cannula 852 relative to the bottom surface 812 of the base structure (and, thus, relative to the patient-user's skin or surface of other subject to be injected). That angle may be any suitable angle that is not perpendicular or parallel to the bottom surface 812 of the base structure (and, thus, relative to the patient-user's skin or surface of other subject to be injected). In one example embodiment, the angle is within the range of about 10° to about 80° (or 100° to 150°) and in a particular embodiment is about 45° (or 135°).

With the needle or cannula 852 inserted into the patient-user's skin (or surface of other subject), the device 850 (including the needle or cannula 852) may be withdrawn from the slide structure 808 and remain on the patient-user's skin (or surface of other subject). After the cap structure 804 and base structure 802 have been moved to the insert position and the device 850 has been withdrawn from the slide structure 808, the slide structure 808 may be withdrawn back into the interior of the base structure 802, toward the retracted position, for example, by returning the cap structure 804 and the base structure 802 to the retracted position (FIGS. 26a, 26b and 26c). In particular embodiments, a needle may be coupled to the slide structure 808 and retracted with the slide structure 808, leaving a hollow cannula (and other structure, such as a sensor structure) in place on the patient-user's skin (or surface of other subject). In other embodiments, the needle and cannula may be inserted as a set by the needle inserting device 800 and the needle may be removed from the cannula at a time after completion of the operation of the needle inserting device 800.

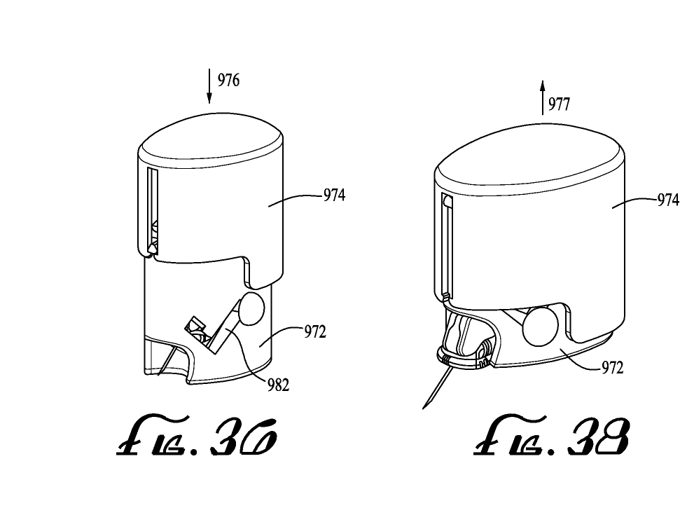

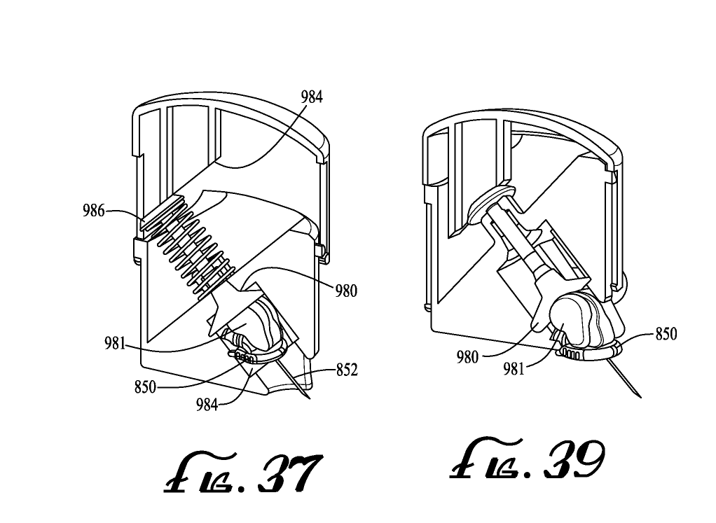

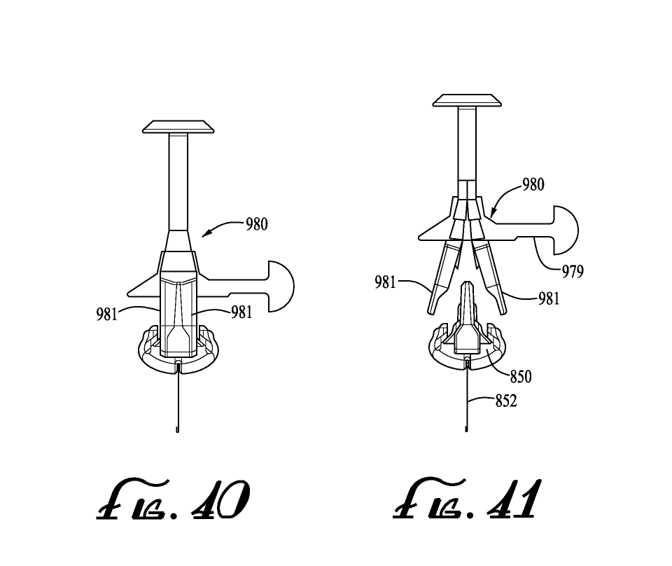

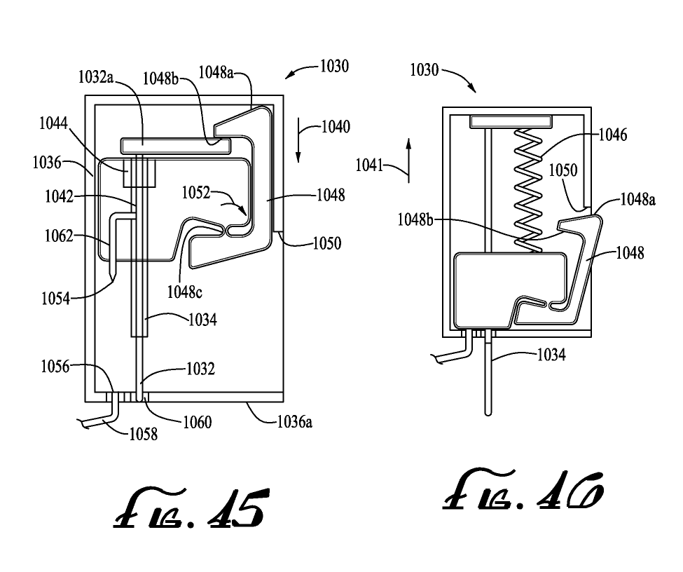

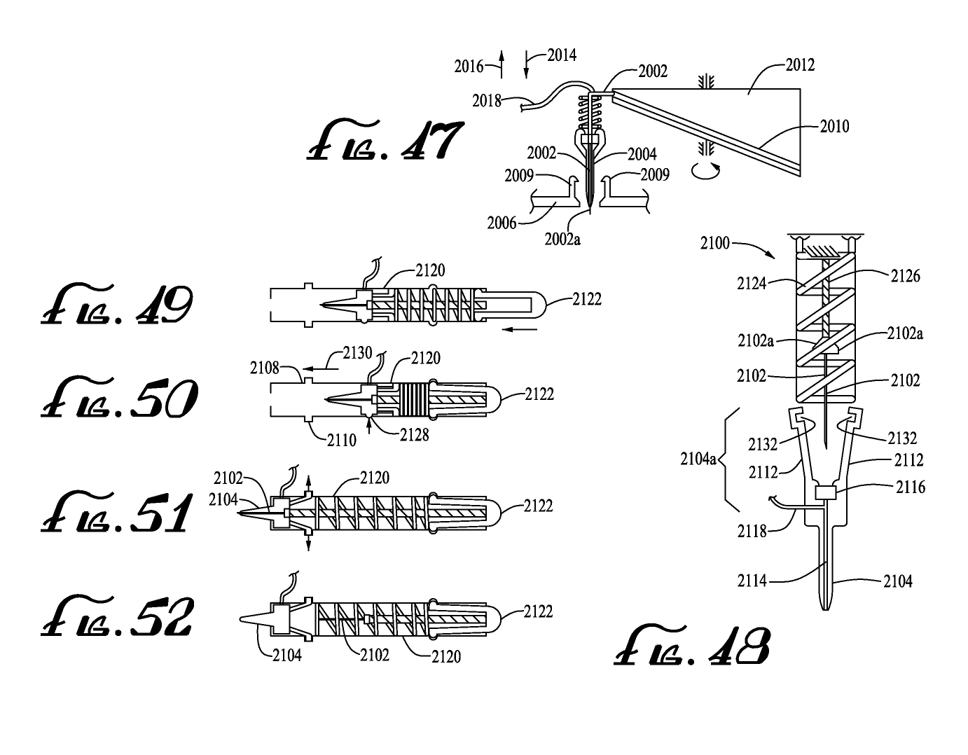

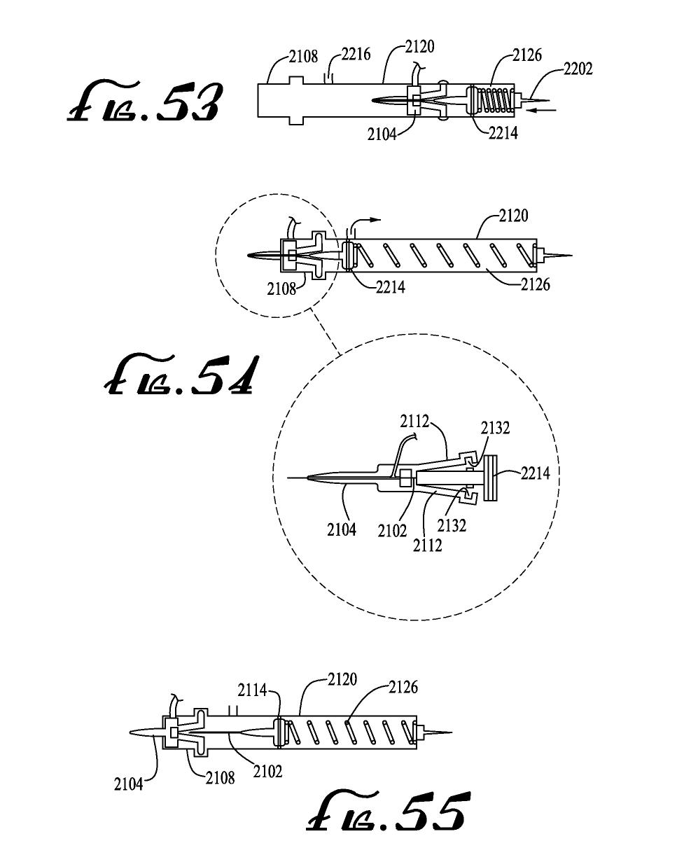

Another embodiment of a needle inserting device 900 is shown in FIGS. 28 to 33, for converting a force directed generally perpendicular to the patient-user's skin (or surface of other subject to be injected) into an angled insertion force for inserting a needle or cannula at an angle (a non-perpendicular angle) to the patient-user's skin (or surface of other subject). The device 900 is shown in a retracted position in FIGS. 28 and 29 and in an insert position in FIG. 30.

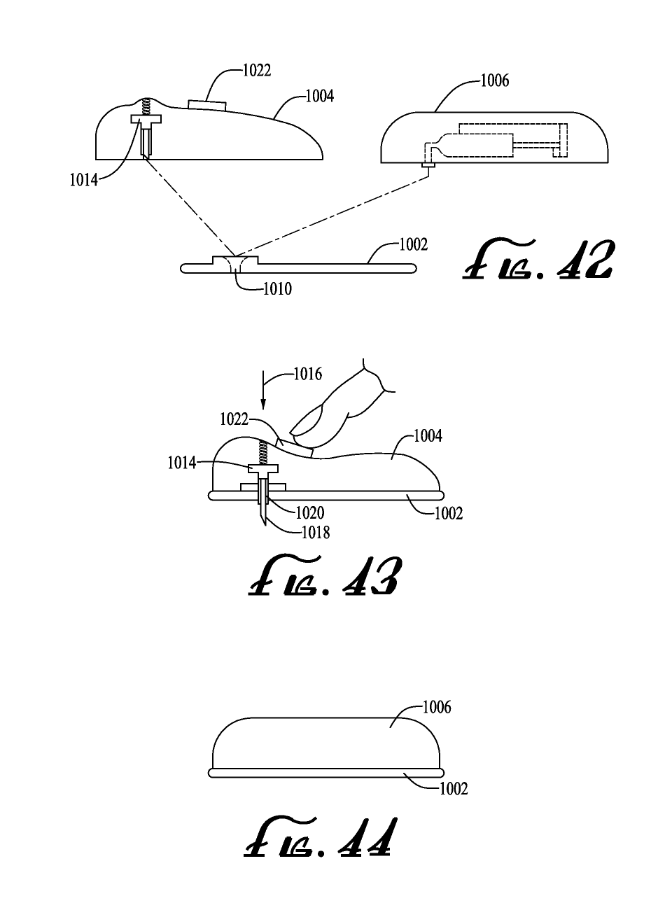

The device 900 includes a base structure 902 and a cap structure 904 that is supported by the base structure 902 for movement relative to the base structure 902 in the direction of arrows 906 and 907. Cross-section and partial views of the device 900 are shown in FIGS. 31-33, to illustrate an example of suitable structure of the device.

The base structure 902 and the cap structure, each has a generally rigid body made of any suitable material, including, but not limited to plastic, metal, ceramic, composite material or the like. The body of the base structure 902 has a pair of tabs 908 and 910 that extend in two opposite directions relative to each other. The tabs 908 and 910 engage a corresponding pair of slots 911 in two opposite side walls of the body of the cap structure 904. Each slot 911 has a longitudinal dimension, extending generally perpendicular to a bottom surface 912 of the base structure 902. The engagement of the tabs 908 and 910 with the slots 912 allow the cap structure 904 to move relative to the base structure 902 in a direction generally perpendicular to the bottom surface 912 of the base structure 902, from a refracted position (FIGS. 28, 29, 31 and 32) to an insert position (FIG. 30).

The base structure 902 supports a first linear gear 914 for movement at an angle (a non-perpendicular angle) relative to the bottom surface 912 of the base structure 902. In the illustrated embodiment, the base structure 902 includes a guide rail 916 on either side of the linear gear 914, having grooves for receiving projections extending from the linear gear. The grooves and projections guide the linear gear 914 in an angled direction of motion relative to the bottom surface 912 of the base structure 902, from a refracted position (shown in FIGS. 28, 29 and 31-33) to an insert position (FIG. 30).

The base structure also supports a rotary gear 918 in operative engagement with the linear gear 914. The rotary gear 918 is support for rotation and has a grooved portion of its length arranged in engagement with grooves on the linear gear 914. The rotary gear 918 has a further grooved portion of its length arranged in operative engagement with grooves on a second linear gear 920. The second linear gear 920 is fixed to the cap structure 904 and moves in a linear motion with the motion of the cap structure 904 (generally perpendicular to the bottom surface 912 of the base structure 902).

A bias mechanism, such as, but not limited to a coil spring or other spring structure, magnets or the like, may be provided within the device 900, to bias the cap structure 904 and base structure 902 toward the retracted position. For example, a coil spring 922 may be arranged between the cap structure 904 and the base structure 902, as described above with respect to the embodiment of FIGS. 300-27. Alternatively, or in addition, the bias mechanism may include a pair of magnets arranged as described above with respect to the embodiment of FIGS. 300-27.

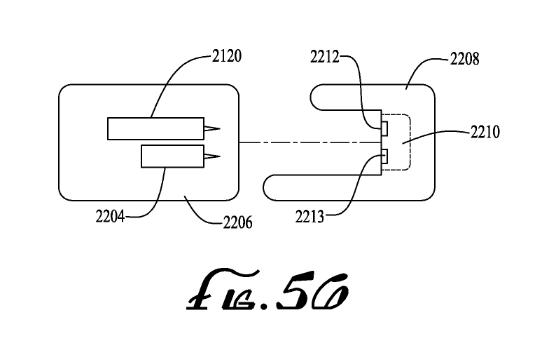

A receptacle structure 924 is connected in a fixed relation to the first linear gear 914. The receptacle structure 924 is configured to receive and retain a device 850 having a cannula or hollow needle 852, as described above. The receptacle structure 924 may have any suitable configuration that is capable of holding and selectively releasing a device having a cannula (or hollow needle) assembly. An example of a receptacle structure is described above with respect to receptacle 820 in FIG. 23c. A further example of a receptacle structure is shown in FIG. 34, wherein the receptacle structure 924 includes a set of three prongs 924a-c that extend from the first linear gear 914.

In operation, the needle inserting device 900 may come pre-assembled or may be assembled as shown in FIGS. 28 and 29. In a retracted position shown in FIGS. 28 and 29, the cap structure 904 is arranged over the base structure 902 and is arranged relative to the base structure 902 at the end of its full range of motion in the direction of arrow 907.

From the retracted position of FIGS. 28 and 29, a patient-user (or a medical practitioner) may place a device 850 in the receptacle 924 of the slide structure 808, to load the needle inserter device. In certain embodiments, the needle inserting device 900 may come from the manufacturer or assembler, pre-loaded and packaged with the device 850 in the receptacle 924, wherein the device 850 may be covered by a removable cover as described above.

In the retracted position, the device 900 may be arranged relative to a patient-user's skin (or surface of other subject to be injected) for injection of the needle or cannula portion 852 of the device 850. In particular, the bottom surface 912 of the base structure 902 may be arranged adjacent and generally parallel to the patient-user's skin (or surface of other subject to be injected) at a desired injection site.

The device 900 is operated to insert the needle or cannula 852 at an angle (a non-perpendicular angle) relative to the patient-user's skin (or surface of other subject to be injected). Prior to insertion of the needle or cannula 852, a peal-sheet 853 may be removed from the sensor structure 850 to expose an adhesive material that will allow the structure 850 to adhere to the patient-user's skin (or surface of other subject), when the structure is brought into contact therewith.

To insert the needle or cannula 852 into the patient-user's skin (or surface or other subject), a force in the direction of arrow 906 is applied to move the cap structure 904 relative to the base structure 902, from the retracted position (FIGS. 28 and 29) toward an insert position (FIG. 30). The force must be sufficient to move the cap structure 904 downward (in the orientation of FIG. 30) relative to the base structure 902, against the force of the bias mechanism 922. The force on the cap structure 904 is applied in a direction generally perpendicular to the bottom surface 912 of the base structure 902 and, thus, generally perpendicular to the patient-user's skin (or surface of other subject to be injected). The force may be applied manually, for example, by the patient-user (or medical technician) pressing downward (in the orientation of FIG. 30) on the cap structure 904 at a desired velocity and timing. Alternatively, the force may be applied by an automated device, in response to an activation signal.

With the relative motion of the cap structure 904 and the base structure 902 in the direction of arrow 906, from the retracted position (FIGS. 28, 29 and 31-33) toward the insert position (FIG. 30), the second linear gear 920 is moved with the cap structure 904 relative to the base structure 902 and rotates the rotary gear 918 about its axis of rotation in the direction of arrow 926. Rotation of the rotary gear 918 in the direction of arrow 926 causes the first linear gear 914 to move, linearly, in the direction of arrow 928. As the first linear gear 914 moves in the direction of arrow 928, the needle or cannula 952 is inserted into the patient-user's skin (or surface of other subject) at a non-perpendicular angle relative to the bottom surface 912 of the base structure 902 (and, thus, at a non-perpendicular angle relative to the patient-user's skin or surface of other subject to be injected). In addition, the exposed adhesive on the device 850 comes into contact with the patient-user's skin (or surface of other subject) and adheres the device 850 to the patient-user (or other subject).

Once the needle or cannula 852 is inserted into the patient-user's skin (or surface of other subject), the device 850 may be removed from the receptacle structure 924. In certain embodiments, the needle may be secured to the receptacle structure 924 and may be automatically withdrawn from a cannula by releasing the force on the cap structure 904 and allowing the bias mechanism 922 to return the cap structure 904 to the retracted position relative to the base structure 902 and, thus cause the linear gear 914 to move in the direction opposite to the direction of the arrow 928.

The angle of the first linear gear 914 (and the angle of the guide rails 914 and 916) relative to the bottom surface 912 of the base structure 902 defines the angle of insertion of the needle or cannula 852 relative to the bottom surface 912 of the base structure (and, thus, relative to the patient-user's skin or surface of other subject to be injected). That angle may be any suitable angle that is not perpendicular or parallel to the bottom surface 912 of the base structure (and, thus, to the patient-user's skin or surface of other subject to be injected). In one example embodiment, the angle is within the range of about 10° to about 80° (or 100° to 150°) and in a particular embodiment is about 45° (or 135°). Accordingly, with the device 900, a force in a direction of the arrow 906 and generally perpendicular to the patient-user's skin (or surface of other subject to be injected) results in an insertion of a needle or cannula 852 at an angle (a non-perpendicular angle) to the patient-user's skin (or surface of other subject).

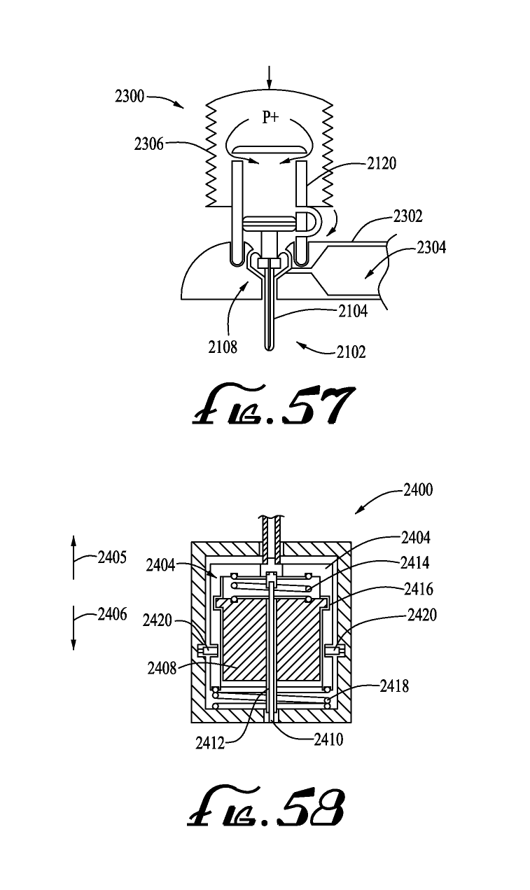

In a further embodiment shown in FIG. 35, a needle inserting device 950 has a structure and operation similar to the device 900 in FIGS. 28-33. However, instead of a set of gears 914, 918 and 920 to transfer a generally perpendicular motion of the cap structure 954 relative to a base structure 952 into an angled insertion motion, the embodiment in FIG. 950 employs a pivoting link structure. In particular, at least one link rod 956 is connected at a first pivot point to the cap structure 954 and at a second pivot point to a slider 958. A receptacle for receiving and holding a device 850 with a needle or cannula 852, as described above, is provided on the slider 958.

The slider 958 engages and moves relative to the grooves one or more guide rails 960 (similar to guide rails 914 and 916 of the base structure 902 described above), to move a device 850 (including a needle or cannula 852) at an angle (defined by the angle of the guide rail 960) to an insert position. After insertion of the needle and cannula 852, the needle may be retracted, leaving the cannula and device 850 in place on the patient-user's skin (or surface of other subject), for example, by returning the cap structure 904 to its retracted position relative to the base structure 902. Upon retraction of the needle, the needle may be removed from the receptacle on the slider 958.

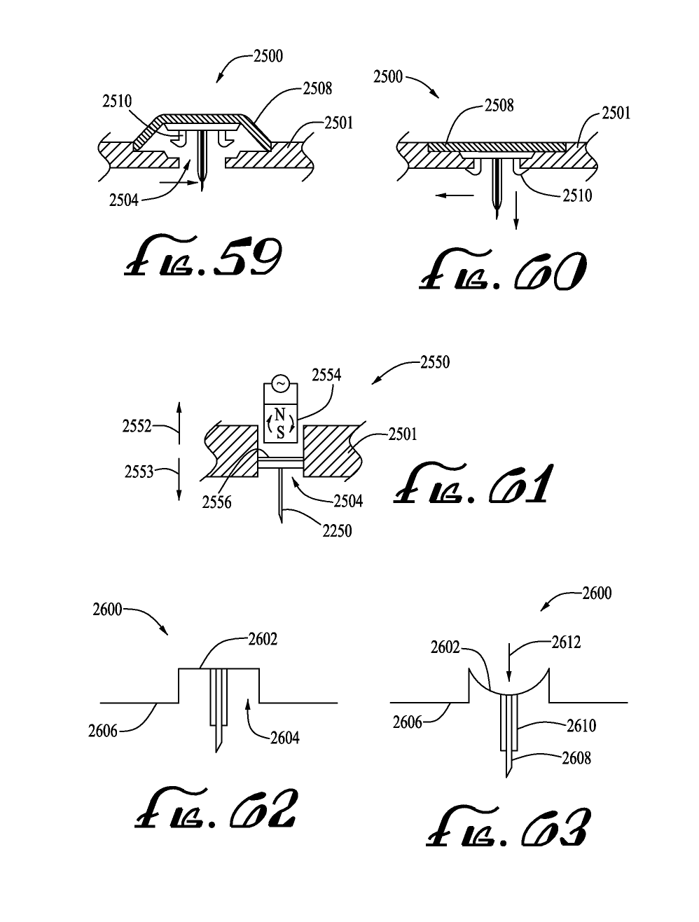

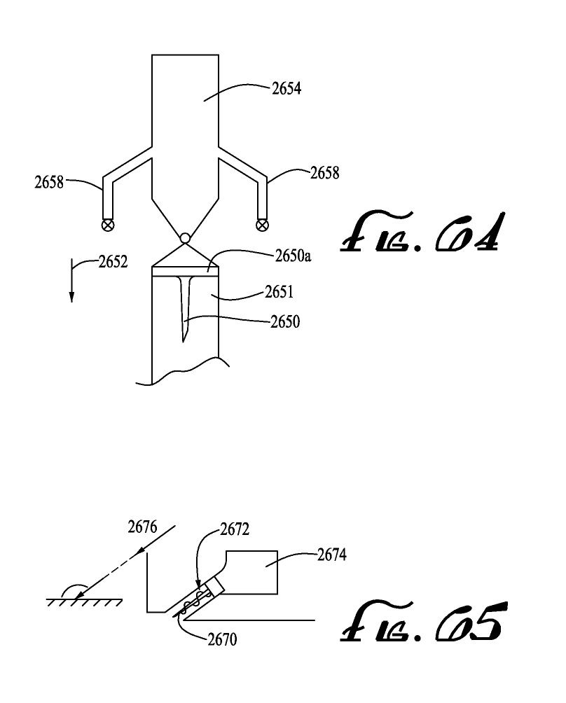

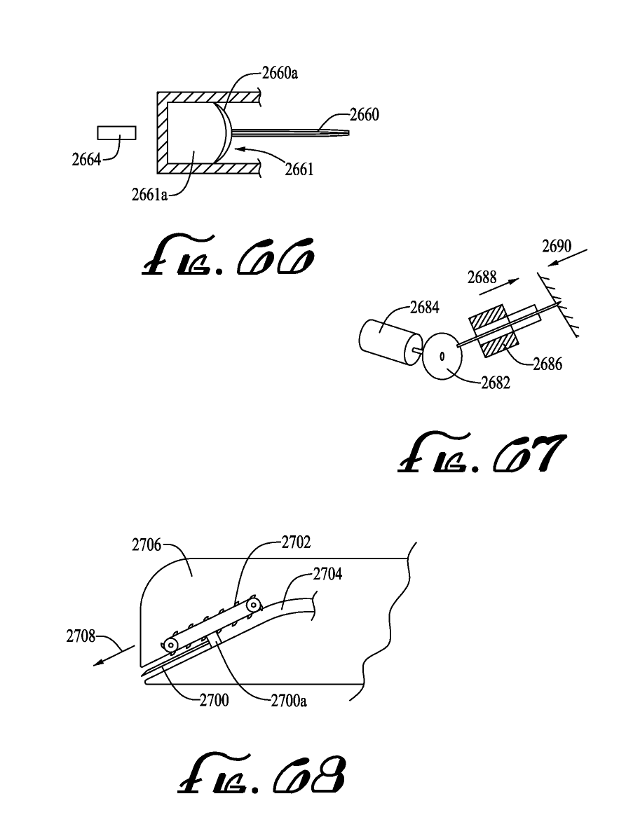

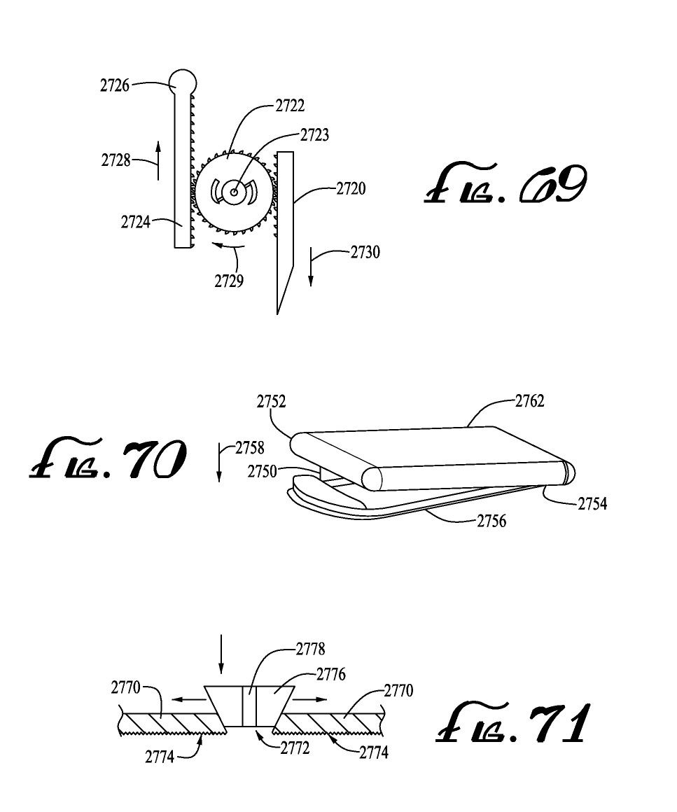

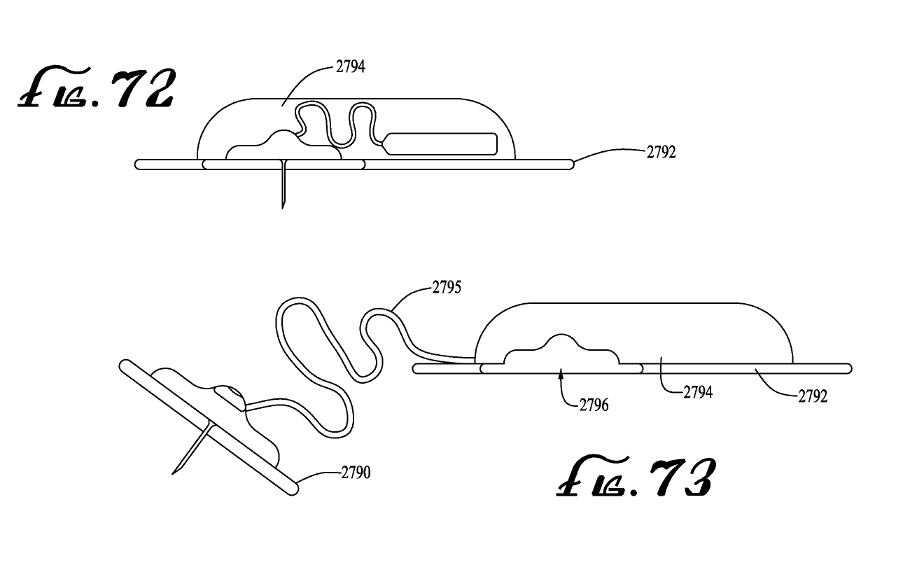

Further embodiments may employ other arrangements of angled slots, gears, pivoting links or the like to transfer a generally perpendicular motion of the cap structure relative to a base structure into an angled needle insertion motion. For example, another embodiment of a needle inserting device 970 is shown in FIGS. 36 to 41, for converting a force directed generally perpendicular to the patient-user's skin (or surface of other subject to be injected) into an angled insertion force for inserting a needle or cannula at an angle (a non-perpendicular angle) to the patient-user's skin (or surface of other subject). The device 970 is shown in a retracted position in FIGS. 36 and 37 and in an insert position in FIGS. 38 and 39. The receptacle of the device 970 is shown in FIG. 40 in a retracted position and is shown in FIG. 41 in an insert position.

The device 970 includes a base structure 972 and a cap structure 974 that is supported by the base structure 972 for movement relative to the base structure 972 in the directions of arrows 976 and 977. The base structure 972 has a bottom surface 978 (relative to the orientation of FIGS. 36-39) that may be placed adjacent and generally parallel to a patient-user's skin (or surface of other subject to be injected), when the device 970 is in the retracted position (FIGS. 36 and 37). A force may be applied to the cap structure 974 in the direction of arrow 976, as described above, to move the cap structure 974 relative to the base structure 972, in the direction of arrow 976.

As the cap structure 974 moves in the direction of arrow 976 relative to the base structure 972, the cap structure 974 engage an arm 979 that extends from a needle device holder 980 located within the base structure 902. The base structure 902 includes an angled slot 982 through which the arm 979 extends. The base structure 902 also includes an angled channel 984 that provides a receptacle for receiving and holding a device 850 with a needle or cannula 852, as described above.

The needle device holder 980 includes two or more moveable jaws 981 at an end of shaft 982, where the jaws 981 may be moved together to clasp the device 850 between the jaws and may be moved apart to release the device 850. The jaws 981 may be biased toward an open direction by a natural spring force of the material that the holder 980 is made from and/or by bias springs or other bias structure included with the holder 980. The needle device holder 980 also includes a hood structure 982 that is slidable along the shaft to an extended position (FIG. 40) to selectively cover a portion of the jaws 981 and close the jaws 981 onto the device 850 or to a retracted position (FIG. 41) to withdraw from the jaws 981 and allow the jaws 981 to flex open. The hood structure 982 is connected to the arm 979.

Further movement of the cap structure 974 in the direction of arrow 976, after engagement with the arm 979 causes the arm 979 to move along the angled slot 982 and to draw the hood 982 over the jaws 981 to clamp the jaws 981 onto the device 850. As the cap structrure 904 continues to move in the direction of arrow 976, the arm 979 continues to move along the angles slot 982 and to move with the holder 980 to the insert position (FIGS. 38 and 39). Also as the cap structure 974 continues to move in the direction of arrow 976, an angled surface 984 of or in the cap structure 974 contacts a plunger head 986 on one end of the shaft of the holder 980 and forces the shaft of the holder 980 toward the bottom surface 978 of the base structure 972, at a non-perpendicular angle relative to the bottom surface 978. In that manner, the needle or cannula 852 of the device 850 may be inserted into the patient-user's skin (or surface of other subject) at a non-perpendicular angle relative to the patient-user's skin (or surface of other subject). The angle of insertion is defined by the angle of orientation of the shaft of the holder 980 and the angle of the channel 984 in the base structure 972.

After insertion of the needle and cannula 852 of a device 850, the cap structure 974 may be returned to the refracted position (FIGS. 36 and 37), for example, by a bias mechanism 986. In the illustrated embodiment, the bias mechanism 986 is a coil spring arranged as described above. However, in other embodiments, other suitable bias mechanisms may be used, as described above, for biasing the cap structure 974 and base structure 972 toward the retracted state. As the cap structure 974 returns to the retracted state, the holder 980 also may be returned to the retracted state, wherein the hood 979 is withdrawn from the jaws 981 and allow the jaws to release the device 850 in its inserted state. The bias mechanism 986 may be arranged to impart a bias force on the plunger head 986 to urge the holder toward the retracted position.

### Multi-Piece Delivery Devices:

Various embodiments of multi-piece infusion medium delivery devices are described in U.S. patent application Ser. No. 11/645,435, filed Dec. 26, 2006, (attorney docket no. 047711.0406), titled “Infusion Medium Delivery system, Device And Method With Needle Inserter And Needle Inserter Device And Method,” (assigned to the assignee of the present invention and incorporated herein by reference in its entirety). Such devices may include a first housing portion (which, in particular embodiments, may be a durable housing portion) for containing components that do not normally come into contact with the patient-user or infusion medium, during operation, such as, but not limited to control electronics, drive devices, power sources and the like. Such devices may also include a second housing portion (which, in particular embodiments, may be a disposable housing portion) for containing components that do normally come into contact with the patient-user or infusion medium during operation, such as, but not limited to, a reservoir.

Some of such multi-piece devices include a separate base member that may be adhered to a patient-user's skin (or surface of other subject to be injected) or otherwise carried by the patient-user, where the first and second housing portions are configured to connect together and to the base, for operation. Other of such multi-piece devices include a base portion that is part of the first or the second housing portion. Some of such multi-piece devices include injection site structure that is incorporated with the base and/or with one or the other of the first and second housing portions. Yet other of such multi-piece devices include an injection site module that contains injection site structure and is connected in fluid-flow communication with one or the other of the first and second housing portions or the base.

In any of those embodiments, a needle inserting device may be incorporated within or connectable to the injection site structure. Various examples of needle inserting devices that may be incorporated or connected to injection site structure is described in the present disclosure and in U.S. patent application Ser. No. 11/645,435, titled “Infusion Medium Delivery System Device And Method With Needle Inserter And Needle Inserter Device And Method” (assigned to the assignee of the present invention), which is incorporated herein by reference.

A further example of a multi-piece needle inserting device 1000 is describe with reference to FIGS. 42-44. Referring to FIG. 42, the multi-piece device 1000 includes a base structure 1002, an inserting device housing 1004 and a pump housing 1006. The base structure 1002, inserting device housing 1004 and pump housing 1006, each may be made of any suitably rigid material, including, but not limited to plastic, metal, ceramic, composite material or the like. The base structure 1002 is configured to be secured to a patient-user's skin (or surface of other subject to be injected) at a desired injection site. The inserting device housing 1004 may be secured to the base structure 1002 either before or after the base is adhered to the patient-user (or other subject), as shown in FIG. 43.

The inserting device housing 1004 includes a needle inserting device 1008, such as, but not limited to any suitable inserting device as described in the present disclosure or in U.S. patent application Ser. No. 11/645,435, titled “Infusion Medium Delivery System Device And Method With Needle Inserter And Needle Inserter Device And Method” (assigned to the assignee of the present invention), which is incorporated herein by reference. When the inserting device housing 1004 is secured to the base structure 1002, as shown in FIG. 43, the needle inserting device 1008 aligns with a needle insertion channel or opening 1010 in the base and may be operated to inject a needle or hollow cannula into the patient-user's skin (or surface of other subject to be injected).

Upon injecting a needle or cannula, a hollow needle or cannula is received and retained in a receptacle portion 1012 of the channel 1010 in the base structure 1002. After injecting the needle or cannula, the inserting device housing 1004 may be removed from the base structure 1002 and disposed of, stored or handled in some other manner, while the base structure 1002 and a hollow needle or cannula remains on the patient-user (or other subject).

After removal of the inserting device housing 1004 from the base structure 1002, the pump housing 1006 may be secured to the base structure 1002, for operation, as shown in FIG. 44. By securing the pump housing 1006 to the base structure 1002, a reservoir in the pump housing 1006 is connected in fluid flow communication with the hollow needle or cannula that has been inserted into the patient-user (or other subject).

In the embodiment of FIGS. 42-44, the inserting device 1008 has a moveable plunger 1014 that is supported for movement within the inserting device housing 1004 moveable in the direction of arrow 1016. A bias member 1017, such as, but not limited to a coil spring or other spring structure, is provided to impart a force on the plunger 1014 to draw the plunger into the inserting device housing 1004, as shown in FIG. 42. A needle 1018 having a sharp tip extends from an end of plunger 1014 and is aligned with the channel 1010 of the base structure 1002, when the inserting device housing 1004 is connected to the base 1002, as shown in FIG. 43. The needle 1018 is moveable in the direction of arrow 1016, with movement of the plunger 1014 in the direction of arrow 1016. A cannula 1020 with a cannula head as described herein may be supported on the needle 1018, for movement with the needle 1018.

The inserting device housing 1004 includes a button 1022 that may be manually operated by a patient-user (or medical technician) to cause the needle 1018 to be inserted into the patient-user's skin (or surface of other subject to be injected). In the illustrated embodiment, the inserting device housing 1004 is formed of a material that provides sufficient resiliency and flexibility to bend under the manual pressure from pressing the button 1022 and push the needle 1018 and cannula 1020 into and at least partially through the channel 1010. As the cannula 1022 is pushed into the channel 1010, the head of the cannula 1020 may engage and be retained by the receptacle 1012 of the channel 1010, for example, by friction fit, snap fit or other suitable retaining or connection arrangement.

Once the cannula 1020 has been received and retained by the receptacle 1012, the patient-user (or medical technician) may stop pressing the button 1022 and allow the inserting device housing 1004 to resiliently return to its original shape. In addition, the bias member 1017, such as, but not limited to a coil spring or other spring configuration, may be provided to draw the plunger 1014 back toward a retracted position (of FIG. 42) to draw the needle 1018 out of the cannula 1020 and into the housing 1004. The inserting device housing 1004 may then be removed from the base structure 1002, leaving the cannula 1020 in the patient-user (or other subject) to allow connection of the pump housing 1006 to the base structure 1002, as described above. Connection of the pump housing 1006 to the base structure 1002 also provides a connection of a reservoir in the pump housing 1006 to the cannula 1020, to provide a fluid-flow connection between the reservoir and the patient-user (or other subject). Various connectors for connecting a reservoir to a cannula may be employed, including connection structures as described in the present application. The multi-piece configuration of FIGS. 42-44 allow for a simplified injection and reservoir connection procedure.

While the needle inserting device 1008 of the embodiment in FIGS. 42-44 includes a manually movable plunger structure, other needle inserting devices may be used, including those describe in the present application and those described in U.S. patent application Ser. No. 11/645,435, titled “Infusion Medium Delivery System Device And Method With Needle Inserter And Needle Inserter Device And Method” (assigned to the assignee of the present invention), which is incorporated herein by reference. Other examples of needle inserting devices are described with reference to FIGS. 45-70.

### Embodiment of FIGS. 45 and 46

An example of a needle inserting device 1030 is described herein with reference to FIGS. 45 and 46. The needle inserting device 1030 is shown in a retracted position in FIG. 45, in which an introduction needle 1032 and cannula 1034 are located within a housing 1036. In FIG. 46, the needle 1032 and cannula 1034 are in an insert position to be inserted in a patient-user's skin (or surface of other subject to be injected).

The needle inserting device 1030 includes a carriage structure 1038 that is supported for movement by and relative to the housing 1036 in the direction of arrow 1040. The introduction needle 1032 is supported by the carriage structure 1038 and extends through a channel 1042 in the carriage structure 1038 in the direction of arrow 1040. One end of the cannula 1034 is attached to the carriage structure 1038, in fluid-flow communication with the channel 1042. The needle 1032 has a head portion 1032a and a shaft portion that extends from the head portion 1032a through the channel 1042 in the carriage structure 1038. A septum or other seal structure 1044 may be located within the channel 1042, to seal the channel 1042 around the needle 1032, yet allow motion of the needle 1032 in the direction of arrow 1041 relative to the carriage structure 1038.

A bias mechanism 1046 is provided to bias the needle head 1032a in the direction of arrow 1041, relative to the carriage structure 1038. In the illustrated embodiment, the bias mechanism is a coil spring. In other embodiments, the bias mechanism may be any suitable structure for providing a bias force on the needle 1032 in the direction of arrow 1041, including, but not limited to other types of spring configurations, magnet configurations as described herein, or the like.

The carriage structure 1038 has pivotal arm 1048 that has a stop surface 1048a arranged to engage a corresponding stop surface 1050 of or supported by the housing 1036, when the carriage structure 1038 is in the insert position (FIG. 46). The pivotal arm 1048 also stop surface 1048b that engages the needle head 1032a, when the carriage structure 1038 is in the retracted position (FIG. 45), yet disengages the needle head 1032a, when the carriage structure is in the insert position (FIG. 46). In the illustrated embodiment, the pivotal arm 1048 includes a flexible extension of the carriage structure 1038, for example, formed as single, molded unitary structure with the carriage structure 1038 and having a hinge connection portion 1048c that provides a natural spring-like force on the arm 1048 to urge the arm 1048 in the direction of arrow 1052. In other embodiments, a further spring or other bias mechanism may be included on the carriage structure 1038 to bias the arm 1048 in the direction of arrow 1052. In yet further embodiments, the pivotal arm 1048 may include a structure that is attached to the carriage structure 1038 for pivotal motion. In yet other embodiments, a resiliently deformable member may be employed, instead of or in addition to the pivotal arm 1048.

The carriage structure 1038 also has a connection needle 1054 that extends in the direction of arrow 1040 and is inserted through a septum 1056 in the housing 1036 for connection to a fluid-flow channel 1058, when the carriage structure 1038 is in the insert position (FIG. 46). The fluid-flow channel 1058 may be connected in fluid-flow communication with a reservoir, sensor structure, or other suitable fluid containing or processing structure, as described herein.

In operation, the needle inserting device 1030 is arranged in the retracted position (FIG. 45) and is placed with the bottom surface 1036a (relative to the orientation shown in FIG. 45) of the housing 1036 adjacent a patient-user's skin (or surface of other subject to be injected). The needle inserting device 1030 is then activated to cause the carriage structure 1038 to move in the direction of arrow 1040, to the insert position (FIG. 46), at which a portion of the length of the needle 1032 and cannula 1034 pass through an opening 1060 in the housing 1036 and are inserted into a patient-user's skin (or other subject). The carriage structure 1038 may be driven in the direction of arrow 1040, upon activation, by any suitable drive mechanism, including, but not limited to, spring drives, pressure drives, magnet drives motor drives, or the like, as described herein and in other applications incorporated by reference herein. Activation of the drive mechanism may be carried out by any suitable manual, mechanical, automatic, electronic or remote electronic mechanism.

As the carriage structure 1038 moves from the retracted position (FIG. 45) to the insert position (FIG. 46), the needle 1032 and cannula 1034 are inserted into the patient-user's skin (or surface of other subject). At the same time, the connection needle 1054 is inserted through the septum 1056, to make fluid flow connection with the channel 1058. The connection needle 1054 may be a hollow needle structure that is connected in fluid flow communication with the channel 1042, through a connection channel 1062 in the carriage structure 1038.

Once the carriage structure 1038 has reached the insert position (FIG. 46), the pivotal arm (or resiliently deformable structure) 1048 aligns with an opening, indentation, or other discontinuity 1062 in the housing 1036, to allow the flexible arm (or resiliently deformable structure) 1048 to flex outward (deform) to position the stop surface 1048a on the arm 1048 in alignment with the stop surface 1050 on the housing 1036. In that position, the arm 1048 inhibits the carriage 1038 from moving, relative to the housing 1036, in the direction of arrow 1041. In addition, in that position, the arm 1048 releases the needle head 1032a and allows the bias mechanism 1046 to move the needle 1032 in the direction of arrow 1041. As the needle 1032 moves in the direction of arrow 1041 by the action of the bias mechanism 1046, the needle is at least partially withdrawn from the cannula 1034 and a fluid flow connection is made between the cannula 1034 and the channel 1058, through the channel 1042, connection channel 1062 and connection needle 1054. The tension of the bias mechanism 1046 may be selected so as to impart a force on the carriage structure 1038, after the carriage structure 1038 reaches the insert position (FIG. 46), to help maintain the connection of the connection needle 1054 with the channel 1058.

In other embodiments, instead of a connection needle 1054 and septum 1056, a length of flexible conduit may be provided to connect the channels 1062 and 1058. The conduit may be stretchable and/or provided with sufficient slack to remain connected as the carriage structure 1038 moves between the retraced and insert positions.

### Embodiment of FIG. 47

An example of a needle inserting device 2000 is described herein with reference to FIG. 47. The needle inserting device 2000 operates to insert an introduction needle 2002 and a cannula 2004 into a patient-user's skin (or surface of other subject to be injected), then withdraw the needle 2002 and leave the cannula 2004 in place. The needle inserting device 2000 may be employed with a base structure 2006 (as described above), injection site module housing, or the like, that has a nest 2008 for receiving the head 2004a of the cannula 2004. The base structure 2006 may be placed adjacent a patient-user's skin (or surface of other subject to be injected) while the device 2000 is in a retracted state (as shown in FIG. 47). In that position, the device 2000 may be activated to move the needle 2002 and cannula 2004 to an insert position, in which at least a portion of the length of the needle 2002 and the cannula 2004 are moved through an opening in the base structure, to an insert position, to pierce the patient-user's skin (or surface of other subject). The nest 2008 may include one or more flexible pawls 2009 for retaining the cannula head 2004a in place, when the cannula 2004 is moved to an insert position.

The needle 2002 has a sharp end 2002a that is extended through the catheter 2004. The needle 2002 has a second end 2002b that is operatively connected to a rotary cam. In the illustrated embodiment, the second end 2002b forms a bend (about 90°) and is engaged with a groove 2010 in a rotary cam 2012. The cam 2012 is supported for rotation about a cam axis. The cam 2012 may include a disk-shaped member that has a peripheral edge that is thicker on one side of the axis than the other (when viewed in cross-section, as shown in FIG. 47. The groove 2010 extends along the peripheral edge of the disk-shaped member of the cam 2012, at a non-perpendicular angle relative to the rotation axis of the cam 2012.

The cam 2012 may be coupled to any suitable drive mechanism, for selectively driving the cam 2012 in a rotary motion about the axis of the cam. The drive mechanism may include a pre-wound spring (pre-wound to impart a rotational force on the cam 2012, in an unwinding or winding direction of the spring) coupled to the cam 2012. In other embodiments, other suitable drive mechanisms may be coupled to the cam 2012 for selectively driving the cam 2012, including, but not limited to other spring configurations, drive motors, magnetic drives, or the like.

As the cam 2012 is rotated, the needle end 2002b rides within the groove 2010 of the cam 2012 and translates the rotational motion of the cam 212 into a linear motion of the needle 2002 in the direction of arrow 2014 for insertion of at least a portion of the needle 2002 and the cannula 2004 into a patient-user's skin. Linear motion of the needle 2002 in the direction of arrow 2014 causes the cannula 2004 to move, with the needle 2002, in the direction of arrow 2014, to insert the needle and catheter into the patient-user (or other subject) until the cannula head 2004a engages and is retained within the nest 2008 of the base 2006.

Further rotation of the cam 2012 will result in the needle 2002 being withdrawn, at least partially, from the cannula 2004, leaving the cannula in the nest 2008 (and in the patient-user or other subject). A fluid-flow conduit 2018, such as, but not limited to a flexible tubing, may be connected in fluid-flow communication with the cannula. Accordingly, the device 2000 may be set such that a first part of a full rotation of the cam 2012 causes the needle 2002 and cannula 2004 to be inserted into the patient-user (or other subject) and the next part of the cam rotation causes the needle 2002 to withdraw (at least partially) from the cannula 2004.

### Embodiment of FIGS. 48-52

An example of a needle inserting device 2100 is described herein with reference to FIGS. 48-52. The needle inserting device 2100 operates to insert an introduction needle 2102 and a cannula 2104 into a patient-user's skin (or surface of other subject to be injected), then withdraw the needle 2102 and leave the cannula 2104 in place. The needle inserting device 2100 is shown in a partial exploded view in FIG. 48, in an initial position in FIG. 49, in a loaded position in FIG. 50, in an insert position in FIG. 51 and in a retracted position in FIG. 52.

The needle inserting device 2100 may be employed with a base structure (as described above), injection site module housing, or the like, that has a nest 2108 for receiving the head 2104a of the cannula 2104. The base structure may be placed adjacent a patient-user's skin (or surface of other subject to be injected) while the device 2100 is in a loaded state (as shown in FIG. 50). In that position, the device 2100 may be activated to move the needle 2102 and cannula 2104 to an insert position, in which at least a portion of the length of the needle 2102 and the cannula 2104 are moved through an opening in the base structure, to an insert position, to pierce the patient-user's skin (or surface of other subject). The nest 2108 may include one or more indentations, openings, contours or the like 2110 for engaging one or more flexible arms 2112 on the cannula head 2104a and retaining the cannula 2104 in place, when the cannula 2104 is moved to an insert position (FIG. 51).

The cannula head 2104a has a central fluid-flow channel 2114 through which the needle 2104 may extend, and a septum 2116 arranged to seal the central channel 2114 around the needle 2104. A connection channel 2118 is connected in fluid-flow communication with the channel 2114 and may be further connected in fluid flow communication with a reservoir, sensor or other structure for holding or processing fluid.

The needle inserting device 2100 has a housing 2120 that has a generally cylindrical shape and a hollow interior. The housing 2120 is open on one end of the cylindrical shape to receive a portion of the length of a handle 2122. The housing 2120 is also open on the other end to receive the cannula 2104, with the flexible arms 2112 bent toward each other against their natural (or biased) shape (state) shown in FIG. 48. A compression spring 2124 is located within the housing 2120 and is arranged to impart a force on the cannula 2104, when the device 2100 is in the loaded position (FIG. 50). A retention spring 2126 is also located within the housing 2120 and is connected to a head or hub 2102a of the needle 2102, to retract the needle 2102, when the device 2100 is in the refracted position (FIG. 52). In the illustrated embodiments, the compression spring 2124 and the retention spring 2126 are coil springs. In other embodiments, other suitable spring or bias mechanisms may be used.

In operation, the needle inserting device 2100 may be arranged in an initial position, as shown in FIG. 49, with a cannula 2104 inserted at least partially into one end of the cylindrical housing 2120 and with the needle 2102 extending through the cannula 2104. In the initial position, the cannula may be releasably locked to the housing 2120, for example, by one or more flexible or deformable tabs, protrusions, arms or the like on the cannula 2104, that engage a corresponding opening, indentation, stop surface or the like in the housing 2120. Alternatively, or in addition, the tabs, protrusions, arms or the like may be on the housing 2120 and the opening, indentation, stop surface or the like may be on the cannula 2104. The cannula 2104 may be unlocked from a locked state with the housing 2120 by, for example, applying a suitable manual pressure on the housing (by squeezing the housing) at a release button location 2128 on the housing 2120. In other embodiments, the cannula 2104 may be locked to the housing 2120 in other suitable manners that allow for selective release of the lock, including, other mechanical locking structures and electronic or magnetically operated locks. In embodiments in which the sharp tip of the needle 2102 extends from the housing 2120 when the device 2100 is in the initial position, a removeable cover or cap may be provided over the needle tip and/or the needle end of the housing 2120.

From the initial position (FIG. 49), the device 2100 may be set to a loaded position (FIG. 50), by moving the handle 2122 further into the housing 2120. As the handle 2122 moves toward the loaded position, the compression spring 2124 compresses against its natural length and imparts a force on the cannula 2104 in the direction of arrow 2130.

However, because the cannula 2104 is locked at 2128, the cannula 2104 remains inside of the housing 2120 in the loaded position.

The handle 2122 may be provided with by one or more flexible or deformable tabs, protrusions, arms or the like, that engage a corresponding opening, indentation, stop surface or the like in the housing 2120, when the handle 2122 is moved to the loaded position (FIG. 50). Alternatively, or in addition, the tabs, protrusions, arms or the like may be on the housing 2120 and the opening, indentation, stop surface or the like may be on the handle 2122. Accordingly, the handle 2122 may be locked relative to the housing 2120, when moved to the loaded position. In certain embodiments, a release mechanism, as described above for the cannula 2104, may be provided to selectively release the device 2100 from the loaded position. The handle 2122 may be moved to the loaded position, relative to the housing 2122, for example, by applying a manual pushing force onto the handle, until the tabs, protrusions, arms or the like engage with the opening, indentation, stop surface or the like.

Once the device is in the loaded position (FIG. 50), the device may be arranged with the needle end of the housing 2120 adjacent and aligned with the nest 2108. In that position, the cannula 2104 may be released from its locked state relative to the housing 2120, using any suitable release mechanism as described above. In one embodiment the release may be accomplished manually, by the patient-user (or medical technician). In other embodiments, the release may be accomplished electronically or electromechanically, from a remote device, on an automated basis, or the like.

Upon releasing the cannula 2104 from the loaded position of FIG. 50, the cannula is moved by the decompression action of the spring 2124 to the inserted position (FIG. 51) in which at least a portion of the length of the needle 2102 and the cannula 2104 is inserted into the patient-user's skin (or surface of other subject) and the cannuala head 2104a is moved into the nest 2108. Upon the cannula head 2104a being received in the nest 2108, the arms 2112 of the cannula head 2104a are allowed to flex outward (under their natural or a biasing force) to engage and lock with a corresponding number of openings, indentations, stop surfaces or the like on the nest 2108. As the arms 2112 flex outward, the arms release the needle hub 2102a from a hub receptacle contour 2132 in the arms 2112.

From the inserted position (FIG. 51), the retaining spring 2126 is stretched beyond its natural length and applies a return force in the direction opposite to the arrow 2130 on the needle 2102. Upon release of the needle hub 2102a from the receptacle contours 2132 as the cannula arms 2112 flex outward, the retaining spring 2126 draws needle 2102 at least partially out of the cannula 2104. In particular embodiments, the retaining spring 2126 draws the needle 2102 fully into the housing 2120, to avoid inadvertent contact with the needle 2102, as shown in the retracted position of FIG. 52. Once the needle 2102 has been retracted, the housing 2120 may be separated from the nest 2108, while the cannula 2104 remains in place within the nest.

### Embodiment of FIGS. 53-56

An example of a needle inserting device 2200 is described herein with reference to FIGS. 53-56. The needle inserting device 2200 has a structure and operation that is similar in many respects to the embodiment of FIGS. 1007-1011. Accordingly, like reference numbers are used for like elements in the two embodiments. However, instead of employing a compression spring 2124 to force the cannula toward the insert position (as described in the embodiment of FIGS. 1007-1011), the embodiment of FIGS. 53-55 employ pressurized fluid (such as pressurized air or other gas).

The device 2200 is shown in an initial position in FIG. 53. From the initial state, a source of pressurized fluid may be connected to a fluid inlet 2202 of the housing 2120 to set the device into a loaded position. A pressurized fluid source may be connected to the housing 2120 in any suitable manner. In one example as shown in FIGS. 56, a canister of pressurized fluid 2204 may be held on a support structure 2206 with the housing 2120 of the needle inserting device 2200. The support structure 2206 may be a housing and/or may be included as part of the packaging in which the device 2200 is provided to the patient-user. In the embodiment of FIG. 56, the canister 2204 is operatively connected to the inlet 2202 of the housing 2200 by connecting the support structure 2206 to a port structure 2208. The port structure 2208 includes a fluid flow volume 2210 that is sealed by a pair of septa 2212 and 2213. Upon connecting the support structure 2206 to the port structure 2208, a needle of the inlet 2202 and a similar needle for the outlet of the canister 2204 puncture and extend through the septa 2212 and 2213, respectively, to connect the interior of the canister 2204 in fluid flow communication with the interior of the housing 2120, through the volume 2210 in the port structure 2208.

Once the housing 2120 of the device 2200 has been pressurized, the pressure within the housing 2120 applies a force on a plunger head 2214 that is connected to the needle hub 2102a of the needle 2102. The plunger head 2214 has a seal structure for sealing against the interior surface of the housing 2120. The retaining spring 2126 may be connected to the plunger head 2214.

Once the device 2200 is in the loaded (pressurized) position, the device may be arranged with the needle end of the housing 2120 adjacent and aligned with the nest 2108 described in the embodiment of FIGS. 1007-1011. In that position, the cannula 2104 may be released from its locked state relative to the housing 2120, using any suitable release mechanism as described above. Upon releasing the cannula 2104 from the loaded position, the cannula is moved by the action of the pressurized gas on the plunger 2214 to the inserted position (FIG. 54) in which at least a portion of the length of the needle 2102 and the cannula 2104 is inserted into the patient-user's skin (or surface of other subject) and the cannuala head 2104a is moved into the nest 2108.

Upon the cannula head 2104a being received in the nest 2108, the arms 2112 of the cannula head 2104a are allowed to flex outward (under their natural or a biasing force) to engage and lock with a corresponding number of openings, indentations, stop surfaces or the like on the nest 2108. As the arms 2112 flex outward, the arms release the needle hub 2102a from a hub receptacle contour 2132 in the arms 2112, as described above for the embodiment of FIGS. 1007-1011.

In addition, as the plunger head 2214 moves to the insert position (FIG. 54), the plunger head 2214 passes a fluid outlet 2216 in the housing 2120 and, as a result, the pressurized fluid within the housing 2120 is allowed to escape through the outlet 2216. Once sufficient pressurized fluid is released, the retaining spring 2126 draws the needle 2102 out of the cannula 2104.

In particular, when the device 2200 is in the inserted position (FIG. 54), the retaining spring 2126 is stretched beyond its natural length and applies a return force on the needle 2102. Upon release fluid pressure from the housing 2120 through the outlet 2216 and upon release of the needle hub 2102a from the cannula arms 2112, the retaining spring 2126 draws the needle 2102 at least partially out of the cannula 2104. In particular embodiments, the retaining spring 2126 draws the needle 2102 fully into the housing 2120, to avoid inadvertent contact with the needle 2102. Once the needle 2102 has been retracted (FIG. 55), the housing 2120 may be separated from the nest 2108, while the cannula 2104 remains in place within the nest.

### Embodiment of FIG. 57

An example of a needle inserting device 2300 is described herein with reference to FIG. 57. The needle inserting device 2300 has a structure and operation that is similar in many respects to the embodiment of FIGS. 1012 -1015. Accordingly, like reference numbers are used for like elements in the two embodiments. However, in the embodiment of FIG. 57, the source of pressurized fluid is a hand-operated bellows-like structure. The device 2300 is shown in FIG. 57, in an insert position, in which a needle 2102 and cannula 2104 are inserted into the nest 2108 of a base structure 2302. A fluid-flow channel 2118 connects the cannula 2104 in fluid-flow communication with a reservoir 2304.

The needle inserting device 2300 has a bellows-like structure 2306 (shown in a collapsed state in FIG. 57) that can be collapsed from an expanded state to force air (or other fluid that may be contained within the bellows structure) into one or more openings 2308 in the housing 2120. The bellows-like structure 2306 is connected to the housing 2120, over the openings 2308. The bellows-like structure 2306 may be any suitable flexible container structure that is capable of containing a fluid and flexibly compressing to pressurize the contained fluid. In particular embodiments, the bellows-like structure may be operated manually, by the patient-user (or medical technician), by pressing the bellows-like structure 2306 into a compressed state. In other embodiments, the bellows-like structure may be operated by automated mechanisms.

Compression of the bellows-like structure 2306 forces fluid into the housing 2120 to force the plunger head 2214 toward the insert position, to set the cannula 2104 into the nest 2108 and to release fluid pressure to allow retraction of the plunger head 2214 and needle 2102, similar to the operation of the device 2200 described above with respect to FIGS. 1012-1015. However, in embodiments of FIG. 57 that employ a manually operated bellows-like structure, the patient-user (or medical technician) can have a significant amount of control of the insertion rate and time.

### Embodiment of FIG. 58

An example of a needle inserting device 2400 is described herein with reference to FIG. 58. The needle inserting device 2400 has a housing 2402 that contains and supports a needle carriage structure 2404 for movement in the directions of arrows 2405 and 2406 relative to the housing 2402. The housing 2402 also contains and supports a cannula carriage structure 2408 for movement in the direction of arrow 2406.

The needle carriage structure 2404 may have a cup-like shape and supports an introducer needle 2410 for movement with the needle carriage structure 2404. The cannula carriage structure 2408 is arranged within the cup-like shape of the needle carriage structure 2404 and supports a cannula 2412. A channel extends through the body of the cannula carriage structure 2408 and is aligned with the cannula 2412. The needle 2410 extends through the channel in the body of the cannula carriage structure 2408 and through the cannula 2412.

An insertion spring 2414 is arranged between the needle carriage structure 2404 and the cannula carriage structure 2408 to provide a rotary insertion force. The cannula carriage structure 2408 includes one or more protrusions that follow one or more spiral grooves 2416 in the needle support structure 2404, to guide the cannula carriage structure 2408 in a spiral insertion motion around the axis of the needle 2410 and cannual 2412. A retraction spring 2418 is provided between the needle support structure 2404 and the housing 2402, to retract the needle support structure 2404 and the needle 2410, after the needle 2410 and cannula 2412 have moved to the insert position.

The device 2400 is shown in FIG. 58 in a refracted state, in which the insertion spring 2414 is wound against its natural state of winding and imparts a rotational force on the cannula carriage structure 2408. In addition, the retraction spring 2418 is compressed against its natural length to impart a force on the needle carriage structure 2404 in the direction of arrow 2405, relative to the housing 2402. However, the needle carriage structure 2404 is locked in place with respect to the housing 2402 by one or more releasable lock mechanisms 2420. The cannula carriage structure 2408 may be locked in place by any suitable releasable locking mechanism (as described herein) and released by manual, automated or electronic operation.

Upon release of the cannula carriage structure 2408, the force of the spring 2414 causes the cannula carriage structure 2408 to rotate along the spiral groove 2416 and move in the direction of arrow 2406 with the spiral groove, to an insert position at which the needle and cannula are extended through an opening in the housing 2404. In the insert position, the spiral groove-following projections on the cannula carriage structure engage one or more lock mechanisms 2420 and unlock the needle carriage structure 2404 from the housing 2402. Once the needle carriage structure 2404 is unlocked from the housing 2402, the retraction spring 2418 is allowed to expand toward its natural length and move the needle carriage structure 2404 and needle 2410 in the direction of arrow 2405 to withdraw the needle 2410 at least partially from the cannula 2412, after insertion of the cannula 2412.

### Embodiments of FIGS. 59-73

Various embodiments of needle inserting device configurations are described with respect to FIGS. 59-73. Such needle inserting devices may be employed in various suitable contexts described herein or in other applications of use.

In the embodiment of FIGS. 59 and 60, a needle inserting device 2500 includes a sheet 2502 of rubber arranged over an opening 2504 in a housing or base structure 2501 and configured for placement adjacent an desired injection site on a patient-user's skin or other subject (as described herein). The rubber sheet 2502 is formed in a generally cup-shape configuration shown in FIG. 59, defining a convex surface on one side of the sheet (the side facing away from the opening 2504 and a concave surface on the other side of the sheet facing toward the opening 2504). The rubber sheet 2502 is resiliently flexible in that a force may be applied in the direction of arrow 2506 (for example, by manually pressing onto the upper surface of the sheet 2502) to deform the sheet to an insertion state at which the sheet takes the shape shown in FIG. 60. Upon release of the force on the sheet 2502, the sheet 2502 reverts to its cup-like shape shown in FIG. 59. While the sheet 2502 in FIGS. 59 and 60 is described as being made of rubber, other embodiments may employ any suitably flexible, resilient material, such as, but not limited to rubber, plastic, metal, composite material or the like, that is capable of flexing from a predefined shape and returning to the predefined shape under its own resiliency.

The needle inserting device 2500 also includes a cap structure 2508, attached to the concave surface of the sheet 2502. The cap 2506 includes a head portion 2508a that has a shape and size sufficient to cover the opening 2504, upon the sheet 2502 being forced to the insertion state shown in FIG. 60. The base 2501 may include a recess for receiving the head 2508a, when the sheet 2502 is in the insert state (FIG. 60). The cap structure 2508 also includes one or more pawls 2510 or other suitable locking mechanisms for locking the cap structure 2508 to the base 2501, upon the sheet 2502 being forced to the insert state (FIG. 60).

The cap structure 2508 also supports a hollow needle 2512 for movement between a retracted state (FIG. 59) and an insert state (FIG. 60). In the retracted state (FIG. 59), the needle 2512 is located at least partially within the cup-shaped configuration of the sheet 2502 and either does not extend through the opening 2504 or extends a small distance through the opening 2504. In the insert state (FIG. 60), the needle 2512 is more fully extended through the opening 2504. A suitable fluid-flow channel (not shown) may be connected in fluid-flow communication with the needle 2512, either prior to insertion or upon movement of the cap 2506 to the insert position (FIG. 60), for example, for connection of the needle 2519 to a reservoir, sensor or other device for holding or processing fluid.

While the embodiment of FIGS. 59 and 60 may be operated by manually pressing the sheet 2502, the device 2500 may be operated by mechanical, electrical or electromechanical mechanisms, as well. Indeed, various manners of applying a force onto a cap structure 2508 to insert a needle through a needle opening in a housing may be employed in other embodiments of the invention, in devices and systems as described above.

For example, in the embodiment of FIG. 61, a needle 2550 is moved in the direction of arrows 2552 and 2553 by selectively energizing an electromagnet 2554 arranged within proximity of the needle 2550. The speed and direction of motion of the needle 2250 may be controlled by controlling the level (strength of the magnetic field generated by the electromagnet) and direction of current supplied to the electromagnet (polarity of the electromagnet. The needle 2250 may be made of a magnetic material or such a material 2256 may be coated or otherwise attached to at least part of the needle 2250. When the electromagnet 2554 is energized in manner to provide a magnetic pole facing the needle 2250 that is the same as the magnetic polarity of the needle 2250, a force is imparted on the needle to move the needle in the direction of arrow 2552, away from the electromagnet 2554. When the electromagnet 2554 is energized in manner to provide a magnetic pole facing the needle 2250 that is the opposite to the magnetic polarity of the needle 2250, a force is imparted on the needle to move the needle in the direction of arrow 2553, toward the electromagnet 2554. Accordingly, the electromagnet 2554 may be operated to control the motion of the needle 2250 in an insert direction (and, in some embodiments, deposit a cannula into a nest as described above) and a withdraw direction.

In the embodiment of FIGS. 62 and 63, a needle inserting device 2600 includes a sheet 2602 of piezoelectric material arranged over an opening 2604 in a housing or base structure 2606 and configured for placement adjacent an desired injection site on a patient-user's skin or other subject (as described herein). The piezoelectric material is a material that expands in at least one dimension, upon application of a suitable electrical signal. The sheet of piezoelectric material 2602 may be coupled to suitable control electronics for providing a suitable electrical signal to the material to cause the sheet 2602 to expand in at least one dimension. At least a portion of the sheet of piezoelectric material may be connected to the base structure 2606 or other suitable structure supported by the base structure 2606, to cause the sheet 2602 to buckle or bow as shown in FIG. 63, upon application of a suitable electrical signal to cause the sheet 2602 to expand. An needle 2608 may be supported by the sheet 2602 and a cannula 2610 may be supported on the needle 2608, for movement in the direction of arrow 2612 as the sheet 2602 is activated to expand and buckle as shown in FIG. 63 (and, in some embodiments, deposit a cannula into a nest as described above).

A similar configuration may employ a bistable spring, instead of a sheet of piezoelectric material. The bistable spring may be flat or first bowed (for example upward in the orientation of the drawing) at a start position, then pushed (for example by manual force) to a further bowed state (for example, bowed downward in the orientation of the drawing) to insert a needle and cannula. The bistable spring may be allowed to return to its flat or first bowed (e.g., bowed outward) state to withdraw the needle from a cannula, after insertion of a needle and cannula.

In the embodiment of FIG. 64, a needle 2650 is moveable within a channel 2651 in a housing or base structure configured for placement adjacent an desired injection site on a patient-user's skin or other subject (as described herein). The needle 2650 includes a needle head 2650a that provides a plunger function for converting a fluid pressure to a linear motion of the needle 2650 in the channel 2651 in the direction of arrow 2652. A source of pressurized fluid 2654 (such as, but not limited to, compressed air or other gas) is coupled to the chamber 2651, through a controllable valve 2656. One or more release vents having release valves 2658 are provided in fluid flow communication with the pressurized fluid source 2654. The needle 2650 may be moved toward in insert position, in the direction of arrow 2651, by opening the valve 2656. After the needle 2650 has moved to the insert position (and, in some embodiments, deposited a cannula into a nest as described above), the valves on the release vent valves 2658 may be opened to release pressure from the channel 2651. The needle may be biased (by a spring or other suitable bias mechanism, not shown) to retract in the direction opposite to the direction of arrow 2652, once the pressure has been released from the channel 2651.

The embodiment of FIG. 65 is a variation of the embodiment of FIG. 64, wherein the insertion angle of a needle 2670 is arranged to be non-perpendicular to the bottom surface of the needle inserting device (and, thus, at a non-perpendicular angle to the skin of the patient-user or surface of other subject to be injected). Also, in the embodiment of FIG. 65, instead of controlling the activation of the needle motion with the opening of a control valve (as in the embodiment of FIG. 64), the needle motion is activated by releasing a releasable lock 2672 that, when locked, holds the needle 2670 from moving. Once the lock 2672 is released, pressurized fluid in the chamber 2674 causes the needle 2670 to move to the insert position in the direction of arrow 2676 (and, in some embodiments, deposited a cannula into a nest as described above). A return spring 2678 may be provided to retract the needle at least partially from the cannula, after insertion. Any or all of these features may be employed in the embodiment of FIG. 64

In the embodiment of FIG. 66, a needle 2660 is moveable within a channel 2661 in a housing or base structure configured for placement adjacent an desired injection site on a patient-user's skin or other subject (as described herein). The needle 2660 includes a needle head 2660a that provides a plunger function for converting a fluid pressure to a linear motion of the needle 2660 in the channel 2661 in the direction of arrow 2662. A portion 2661a of the channel 2661 behind the needle head 2660a may be sufficiently sealed and may contain an expandable gas or other material that expands (or forms an expandable gas) upon selective activation by one or more of a laser source, heat source, electrical source or other radiation source 2664. By imparting a laser, heat, electrical signal, or other radiation onto the material within the chamber portion 2661a the material expands (or forms an expanding gas) to produce a sufficient pressure within the chamber portion 2661 a to move the needle 2660 toward an insert position, in the direction of arrow 2662 (and, in some embodiments, deposit a cannula into a nest as described above). The head 2660a of the needle 2660 may have a generally parabolic shape or other suitable shape for focusing or enhancing heat or other radiation into the chamber portion 2661a.

In the embodiment of FIG. 67, a needle 2680 is moveable by the rotary action of a rotary wheel or cam. In particular, the needle 2680 is connected at one end to a non-circular disk 2682. The non-circular disk 2682 is connected to a drive source 2684, to rotate about an axis of rotation. The drive source may be a drive motor, spring drive or any suitable mechanism for imparting a controllable rotary force on the disk 2682. The needle 2680 extends through a needle guide or holder 2686, such that, as the disk 2682 rotates, the rotary motion of the disk 2682 is converted into a linear motion of the needle in the insert direction of arrow 2688 (and, in some embodiments, deposit a cannula into a nest as described above) and, then a retract direction of arrow 2690. The insertion direction may be selected to be generally perpendicular or at a non-perpendicular angle relative to the patient-user's skin (or surface of other subject to be injected), by selecting the angle of orientation of the holder or guide 2686.

In the embodiment of FIG. 68, a needle 2700 is moveable by a drive force imparted on a needle head 2700a by a belt drive 2702. The needle head 2700 may be moveable within a channel 2704 in a housing or base structure 2706 configured for placement adjacent a desired injection site on a patient-user's skin or other subject (as described herein). The insertion direction may be selected to be generally perpendicular or at a non-perpendicular angle relative to the patient-user's skin (or surface of other subject to be injected), by selecting the angle of orientation of the channel 2705. The belt drive 2702 may include a belt extending around a pair of wheels, one of which may be coupled to a drive source (not shown), such as, but not limited to a drive motor, spring motor, magnetic drive or the like. The belt may have serrations, teeth or other discontinuities that are configured to engage a corresponding set of serrations, teeth or other discontinuities on the surface of the needle head 2700a. The belt may be driven in one direction for moving the needle 2700 toward an insert position, in the direction of arrow 2708 (and, in some embodiments, deposit a cannula into a nest as described above). The belt may be driven in the opposite direction, for withdrawing the needle, for example, and leaving the cannula in the inserted position. The belt speed and, thus, the needle insertion speed, may be controlled with relatively high precision.

FIG. 69 shows a configuration for converting a linear force in one direction to a linear needle inserting force in an opposite direction. In FIG. 69, a needle 2720 has serrations, teeth or other discontinuities along a linear length of the needle and is supported with the serrations, teeth or other discontinuities in engagement with corresponding serrations, teeth or other discontinuities on a rotary wheel or gear 2722. The wheel 2722 is supported for rotation about an axis 2723. A linear shaft 2724 is provided with serrations, teeth or other discontinuities and is also arranged in engagement with corresponding serrations, teeth or other discontinuities on a rotary wheel or gear 2722. The linear shaft 2724 may include a handle 2726 for manual operation or may be connected to a linear drive source. By applying a force on the shaft 2724 in the direction of arrow 2728, the wheel 2722 is caused to rotate about the axis 2724 in the direction of arrow 2729. The rotational motion of the wheel 2722 is transferred to a linear motion of the needle 2720 in the direction of arrow 2730, toward an insert position (and, in some embodiments, to a position to deposit a cannula into a nest as described above). Movement of the shaft 2724 in the direction opposite to the direction of arrow 2728 will cause the needle 2720 to move in a direction opposite to the arrow 2730, to withdraw the needle, for example, at least partially from a cannula. A weight structure may be provided on the wheel 2722, to assist the rotational motion. A torsion spring may be provided on the wheel 2722, to wind as the wheel rotates toward an insert position and impart an force in the opposite direction to automatically withdraw the needle after insertion of a cannula.

FIG. 70 shows a configuration in which a needle 2750 is moved to an insert position, by the pivotal motion of a pivotal arm 2752. The pivotal arm 2752 may be connected at a pivot point 2754 to a housing or base structure 2756 configured for placement adjacent a desired injection site on a patient-user's skin or other subject (as described herein). The pivotal arm 2752 may be biased toward an open position shown in FIG. 70 by any suitable bias mechanism, such as, but not limited to a coil spring, other spring configuration, magnet configuration, or the like. The pivotal arm 2752 may be moved by manual pressure against the force of the bias mechanism, to move the needle 2750 toward an insert position, in the direction of arrow 2758 (and, in some embodiments, to a position to deposit a cannula into a nest as described above). After insertion of the needle and cannula, the pressure on the arm 2752 may be released to allow the arm 2752 to move back to the retracted position (shown in FIG. 70) under the force of the bias mechanism, while the cannula may be left in place in a nest, as described above. In particular embodiments, the pivotal arm 2752 may include a durable housing portion of a multi-piece infusion device, as described in the above-cited patent applications that have been incorporated herein by reference, where the durable housing portion contains one or more of a reservoir, control electronics, a drive device for driving fluid from a reservoir, linkage structure for linking a drive device to a reservoir and a power source for the drive device.

In the above-described embodiments of needle inserting devices, various mechanisms may be employed for activating the device to insert a needle and cannula. In some contexts, a manual activation may be preferred, wherein a patient-user (or medical technician) manually operates a mechanism (pushes a button, moves a lever, compresses a bellows-like structure or the like). In other embodiments, activation may be accomplished by electronic actuators controlled by an electronic switch that may be manually operated, operated by a control program, or the like. Activation may be accomplished by a remote (wired or wireless) device, by a wireless proximity device or the like. In one example embodiment, a needle inserting device may include an electronic, magnetic or other suitable activator that responds to a transmitter located within a defined proximity of the needle inserting device. For example, the needle inserting device may be configured to include a receiver or other electronics, magnetic devices or the like, that respond to a particular hand-held transmitter, magnet or the like (that transmits a particular signal). The needle inserting device may be configured to respond to a detection of the proximity of the hand-held transmitter or magnet (or detection of the proximity over a period of time or a predefined number of detections of the proximity over a defined period of time, such as, but not limited to, three detections of the transmitter within a five second period).

In any of the above embodiments, a skin stretcher structure may be employed in the surface of the housing or base that contacts the patient-user's skin during an needle injecting operation. An example of a skin stretching configuration is shown in FIG. 71, wherein a portion of the housing or base 2770 adjacent a needle insertion opening 2772 is provided with a rough surface 2774 that is designed to frictionally grip the patient-user's skin, when pressed against the skin. The rough surface 2774 may be formed by serrations, grooves and ribs or any suitable pattern of discontinuities that can sufficiently enhance friction between the surface 2774 and the patient-user's skin. The rough surface 2774 may be formed directly on portions of the housing or base or may be provided on pads that are moveably secured to the housing or base.

In any of the above-described embodiments of needle inserting devices, the needle inserter device housing, the base structure and/or other housing structure that contacts the patent-user's skin adjacent a needle opening may be provided with one or more patches of an anesthesia substance to help numb the skin around the injection site. For example, one or more patches, having microneedles directed toward the patient-user's skin may be provided on the bottom surface of the needle inserter device, base structure or other housing that contacts the patient-user's skin adjacent the injection site.

As part of a needle insertion operation or prior to needle insertion, a wedge-shaped (or cone-shaped) member 2776 is inserted into the opening 2772. The width or diameter of the wedge-shaped member is selected, relative to the width or diameter of the opening 2772, so as to allow the wedge shaped member 2776 to engage the edge of the opening 2772 and impart a spreading force on the structure of the housing or base (or pads) 2770 around the opening 2772. The force imparted by the wedge-shaped member is sufficient to move the surface 2774 outward, relative to the center of the opening 2772 and spread or stretch the patient-user's skin at the location adjacent to the opening 2772. The wedge-shaped member 2776 may include a needle channel 2778, that allows the passage of a needle and/or cannula from a needle inserting device. The channel 2778 is arranged to align the needle and/or cannula with a stretched portion of the patient-user's skin adjacent the opening 2772. In this manner, the needle inserting device may operate to insert a needle and/or cannula through a stretched portion of the patient-user's skin, for improved user comfort.

Embodiments of the present invention may be employed in a multi-piece infusion delivery device as described in above-cited applications that have been incorporated by reference in the present application. Such embodiments may include one or more housing portions for containing a reservoir, a drive device, linkage structure, a power source and a needle inserting device. Some embodiments include a separate base structure to which the one or more housing portions may connect. Embodiments may include a needle inserting device that is part of the base structure. In other embodiments, a needle inserting device may be provided in a module that connects to the one or more housing portions and base, through a flexible tubing, to allow the needle inserting device (and, thus, the injection site) to be located apart from the one or more housing portions and base structure.

In the embodiment of FIGS. 72 and 73, an injection site module 2790 is connected to a base 2792 of a housing portion 2794, through a flexible tubing 2794. The base 2792 and housing 2794 include a receptacle region 2996 in which the injection site module 2790 may be stowed for use, storage or shipment. In the embodiment of FIGS. 72 and 73, the injection site module 2790 may be stowed in the receptacle region 2996 and used as an onboard needle inserting device. Alternatively, the injection site module 2790 may be removed from the receptacle region 2996 for use in a location spaced apart from the location at which the base 2792 and housing 2794 may be secured. Thus, the embodiment of FIGS. 72 and 73 provides a flexibility as to the location of the injection site and can be used in contexts in which it is desired to have an injection site at the same location as the base 2792 and housing 2794 or in contexts in which it is desired to space the injection site apart from the base 2792 and housing 2794.

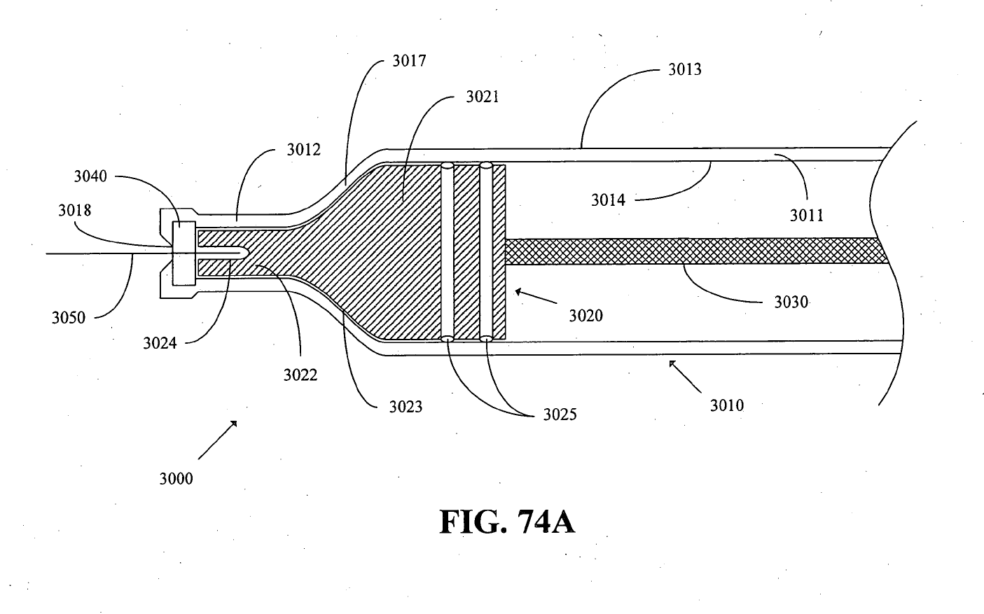

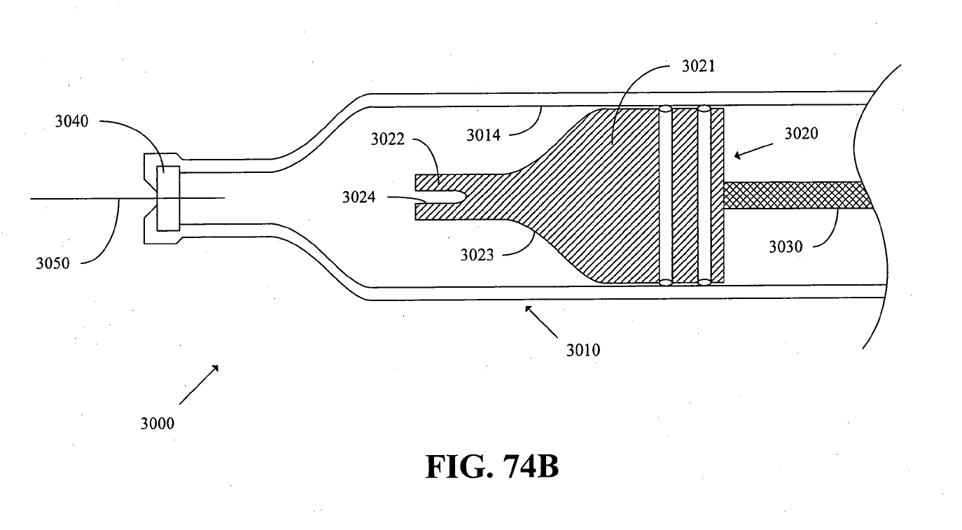

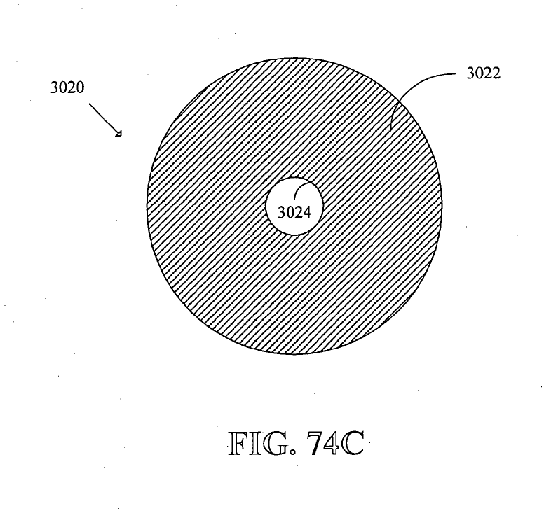

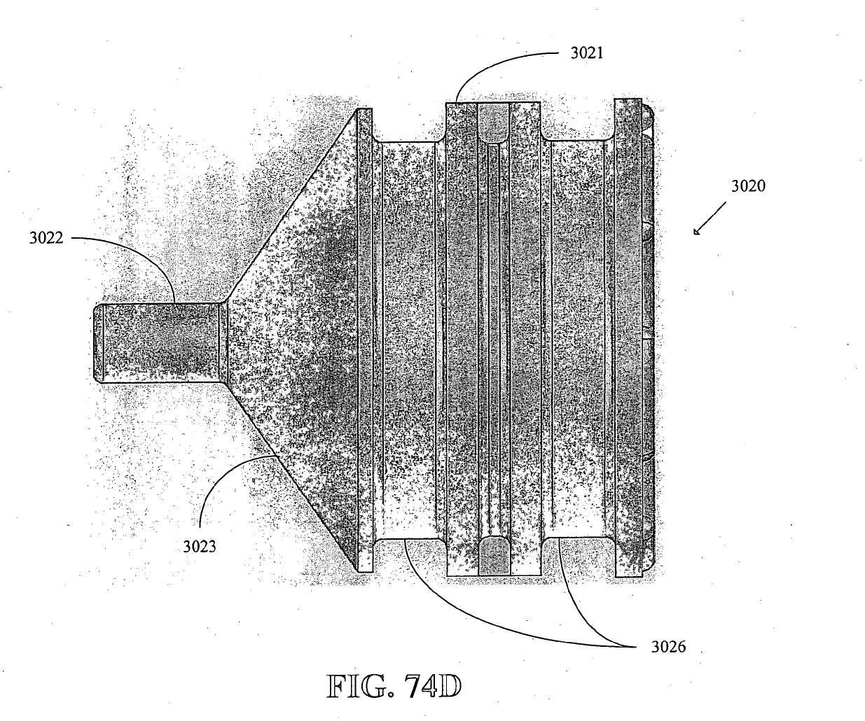

### Embodiments of FIGS. 74

FIG. 74 illustrates an adhesive patch 5400 in accordance with an embodiment of the present invention. The adhesive patch 5400 includes an area 5420 having a certain adhesion capability, and area 5440, area 5450, and area 5470 of increased adhesion capability as compared to the certain adhesion capability of the area 5420.

Disposable medical devices may be attached to a patient's skin. Due to variations in disposable medical devices, skin types, and skin sensitivity levels, sometimes large quantities of adhesive tapes and patches are used to affix a device to the skin, which may lead to excess perspiration, skin irritation, itching, discomfort, and possibly infection. This is especially true of patients with auto-immune deficiencies due to disease states or the administration of certain drug therapies. A medical adhesive with a high adhesion rate proximal to an infusion site, an insertion site, a wound site, or the like, and more breath-ability in areas more distant from such a site, would require a smaller contact area and, thus, may reduce skin irritation, perspiration, and a chance of infection. Such a medical adhesive may also promote device efficacy.

In FIG. 74, area 5430 indicates an infusion site, an insertion site, or the like. The adhesive patch 5400 features the area 5440 of increased adhesion capability around the area 5430. The areas 5440, 5450, and 5470 of the adhesive patch 5400 are merely illustrative of an example of a configuration of increased adhesion capability on an adhesive patch. It should be understood that embodiments of the adhesive patch 5400 are not limited to such an arrangement of areas of increased adhesion capability, but that areas of increased adhesion capability may be positioned in any arrangement on an adhesion patch.

Embodiments of the present invention allow for an adhesive patch, or adhesive tape, featuring areas with increased adhesion capability that ensure that a catheter, a sensor, or other device introduced through a patient's skin will remain in place. Such adhesive patches may allow for reducing an amount of skin coverage of the adhesive patch as compared with an adhesive patch that has only a uniform adhesion capability across the adhesive patch. Thereby, skin irritation and perspiration may be reduced with an adhesive patch having varying levels of adhesion capability in different areas on the adhesive patch, and comfort and wear-ability of a medical device that uses such an adhesive patch may be increased.

An adhesive patch having selective areas of increased adhesion capability may reduce a failure rate of infusion sets by providing increased adhesion capability around an insertion site of a catheter and, thus, helping to prevent the catheter from being partially pulled out an then kinked. Also, such adhesive patches with variable adhesion strength may allow for greater securing of a patch delivery system and minimize the patch footprint on the skin of the patient. Adhesive patches with variable adhesion strength may also allow for greater securing of glucose sensor products to a patient without increasing a patch size. Embodiments of the present invention allow for selective use of augmented adhesives on an adhesive patch.

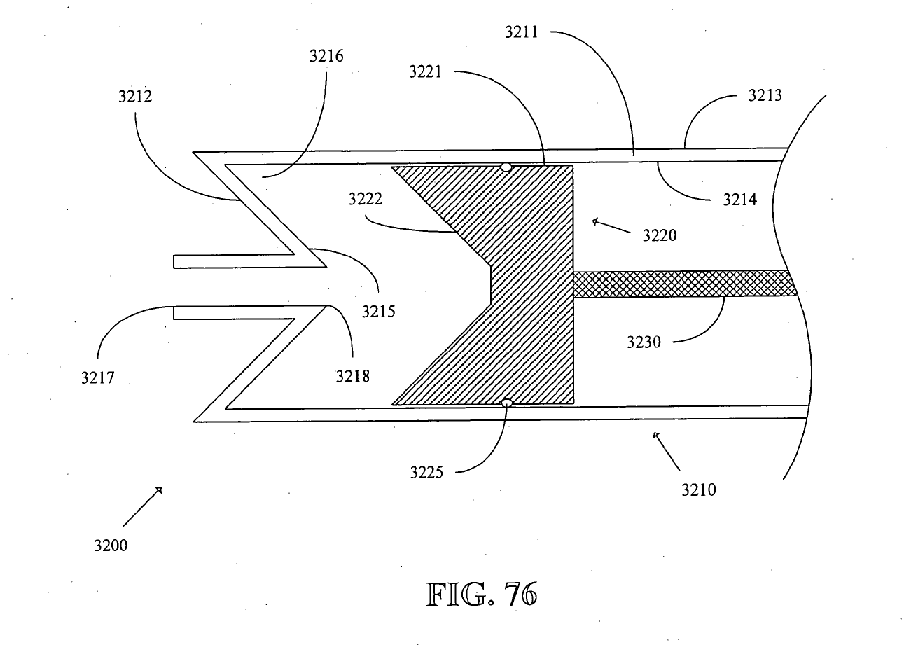

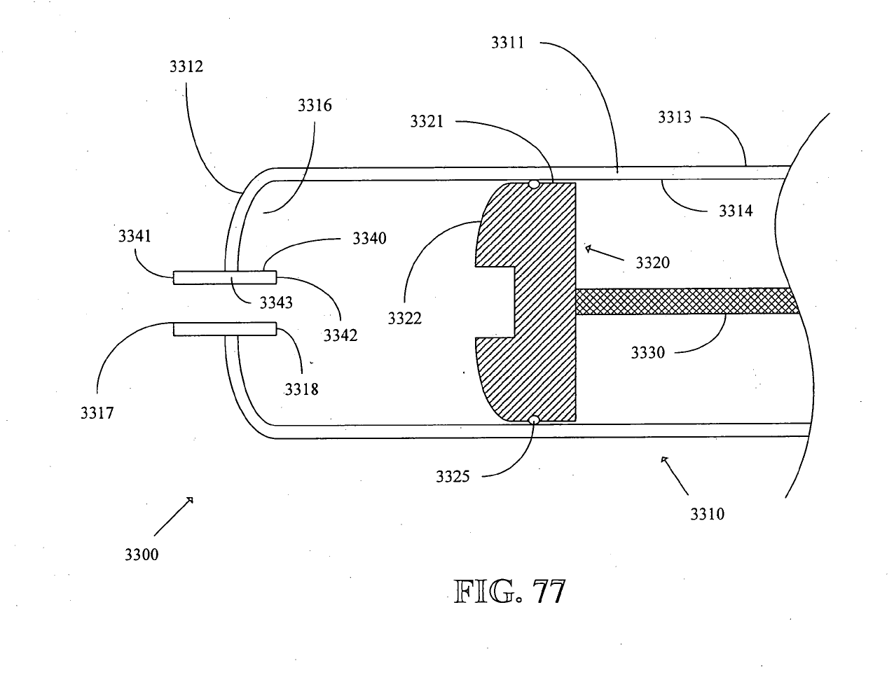

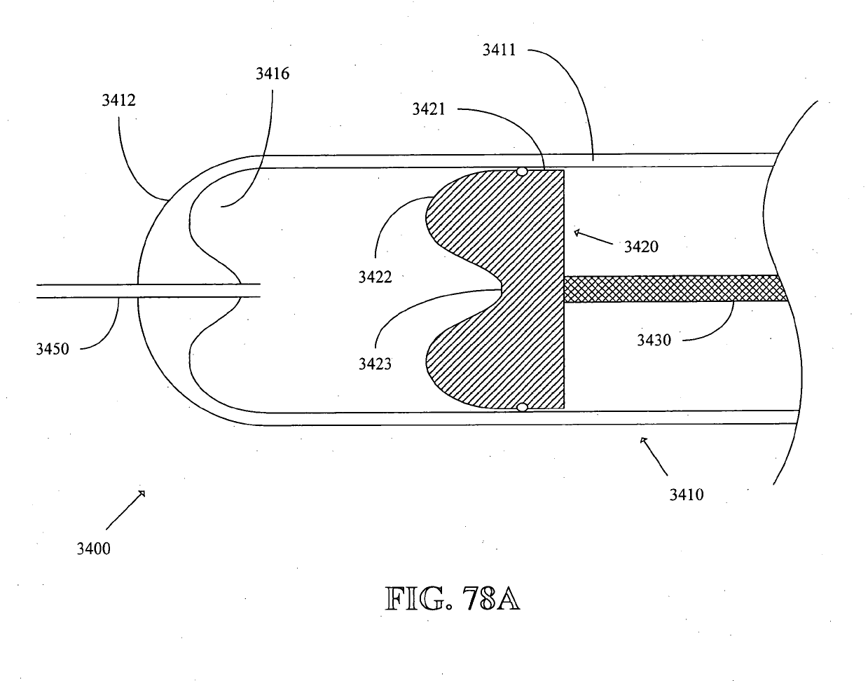

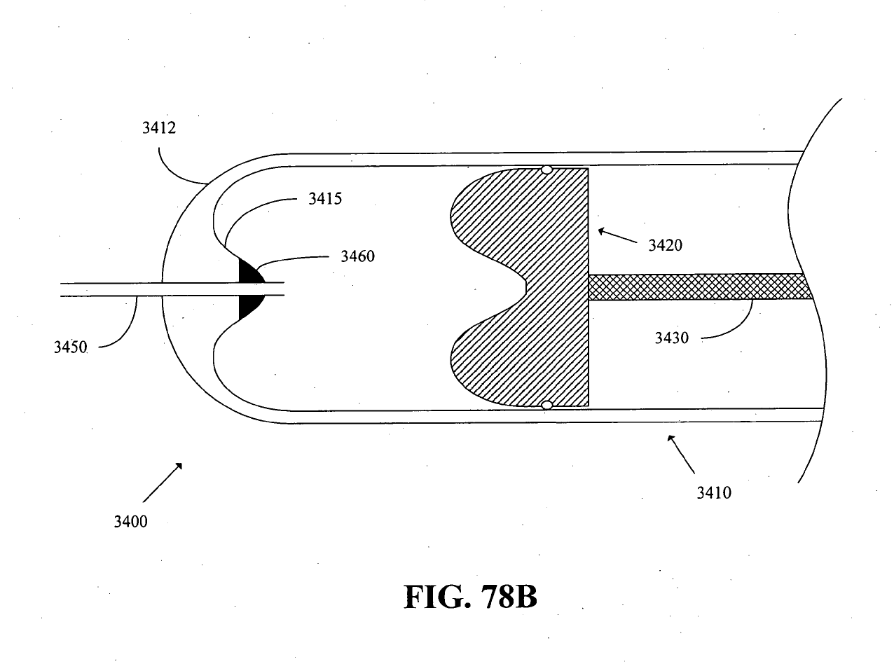

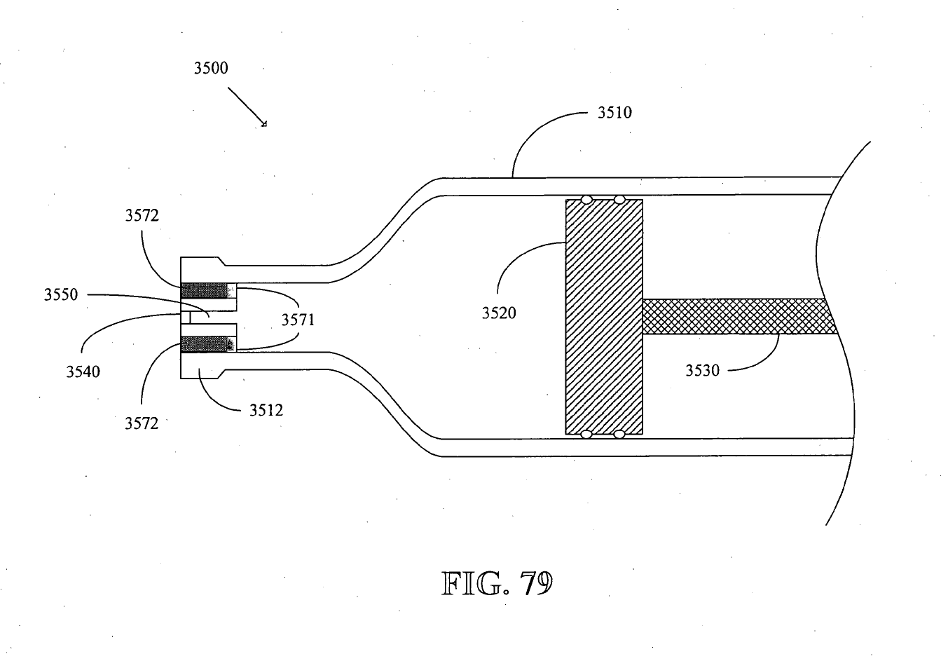

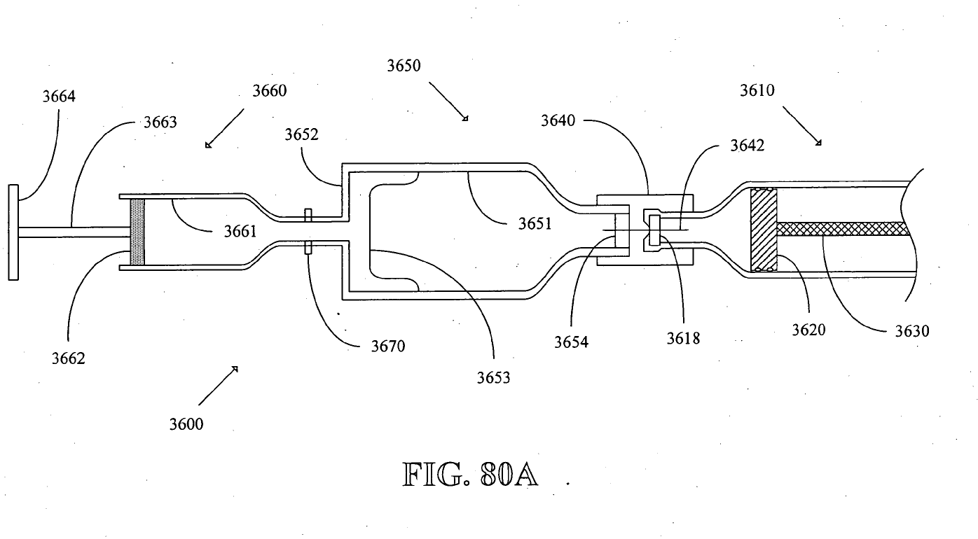

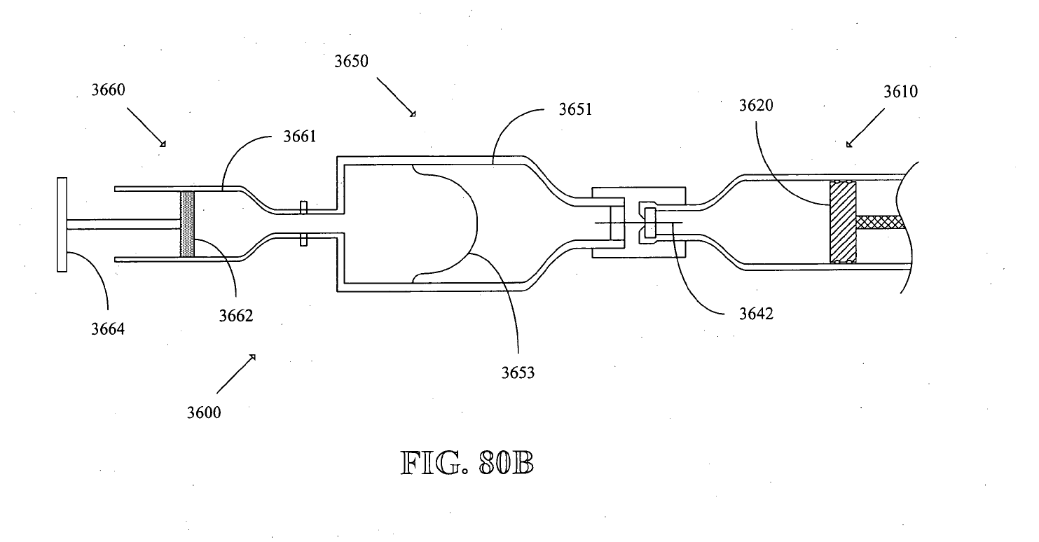

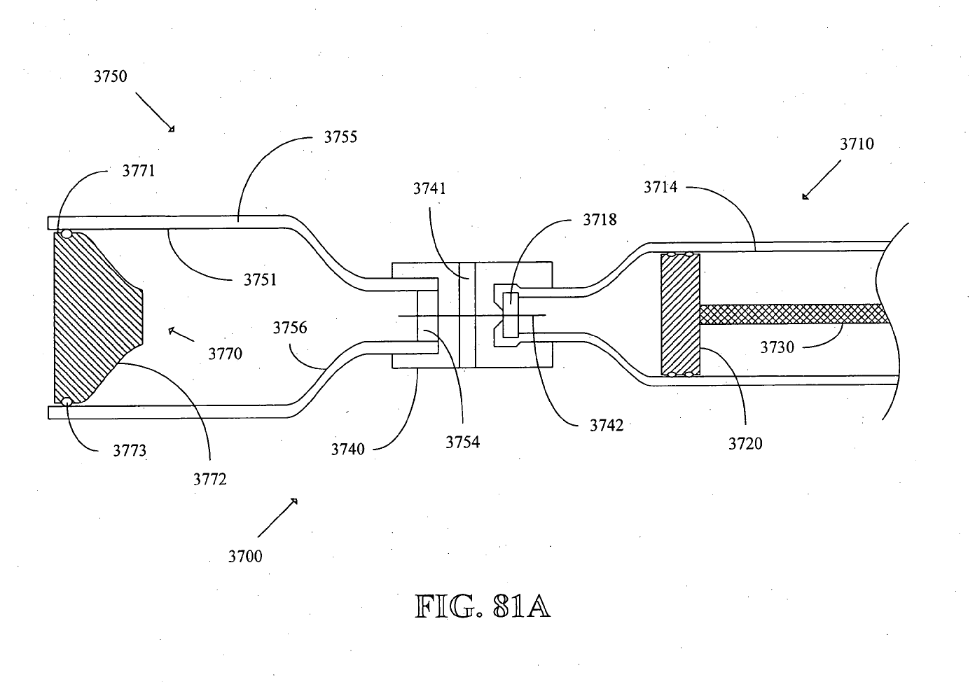

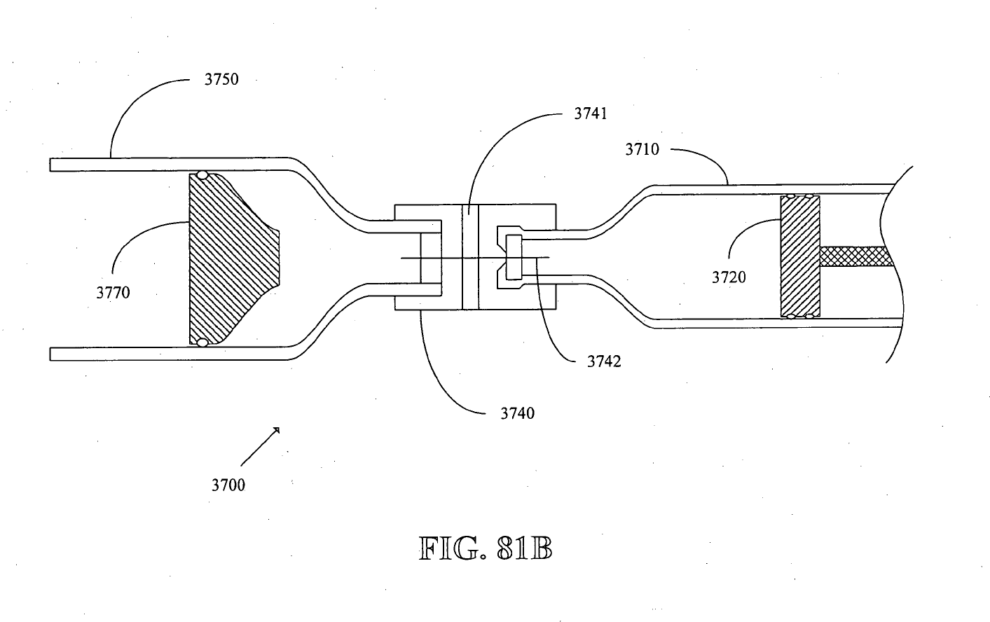

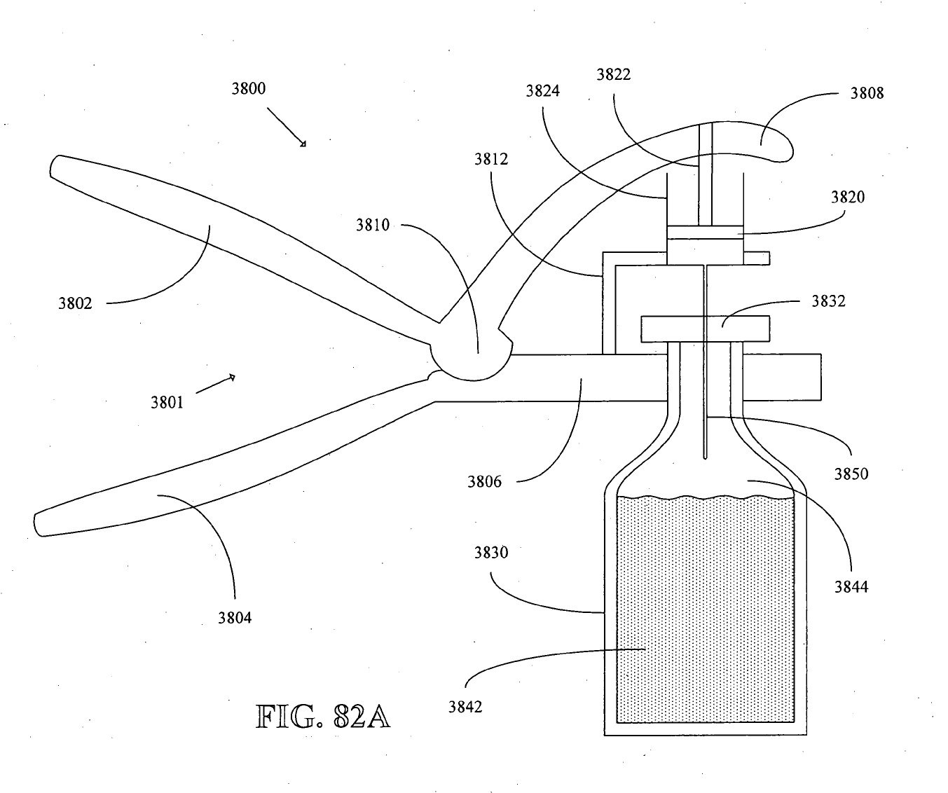

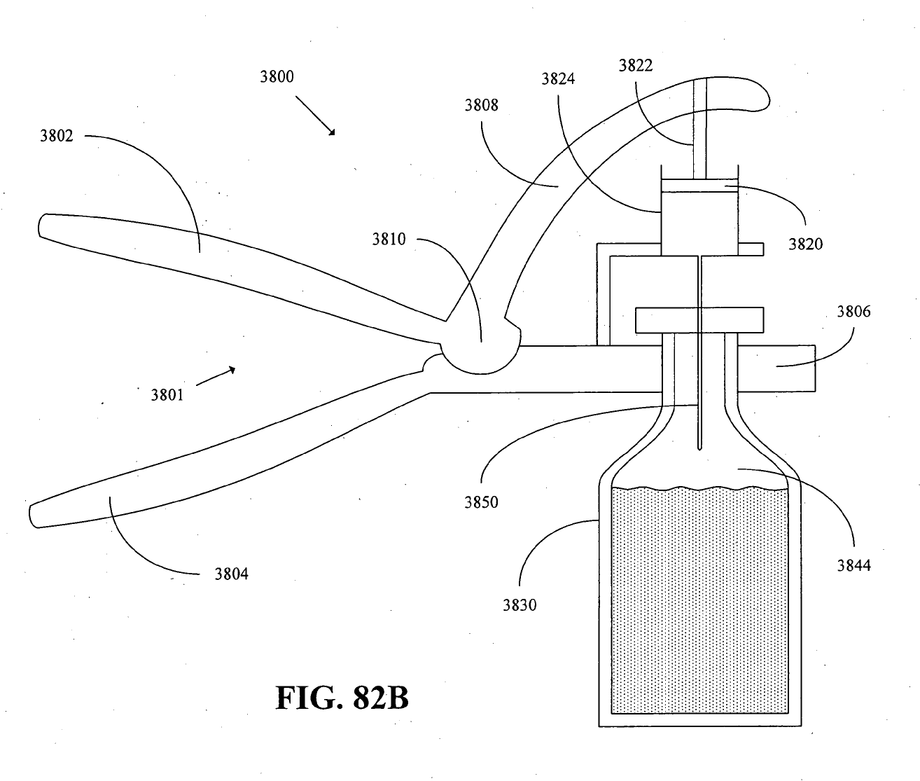

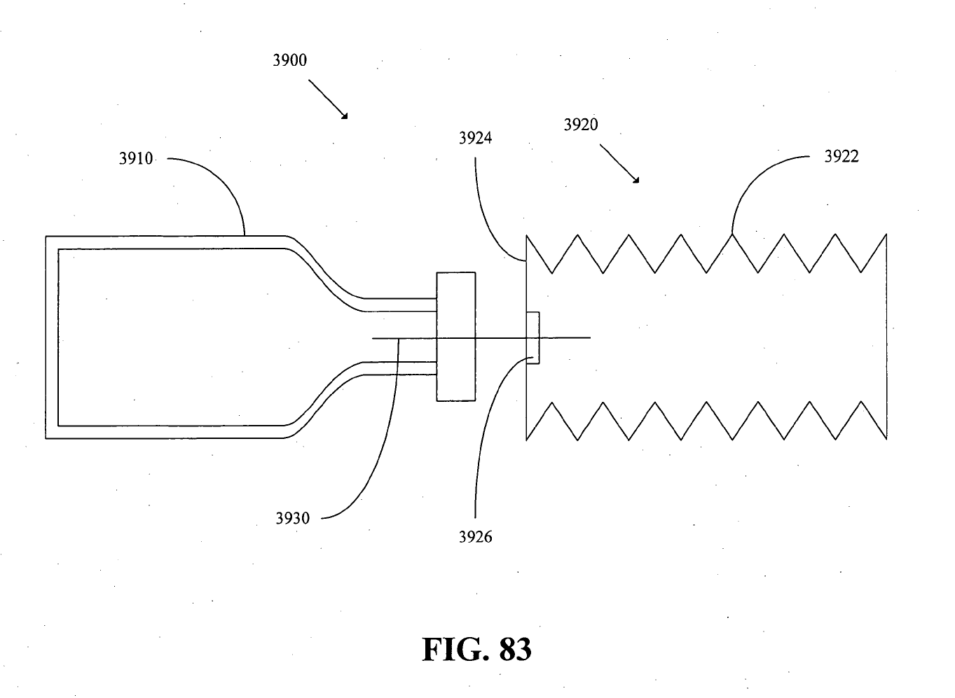

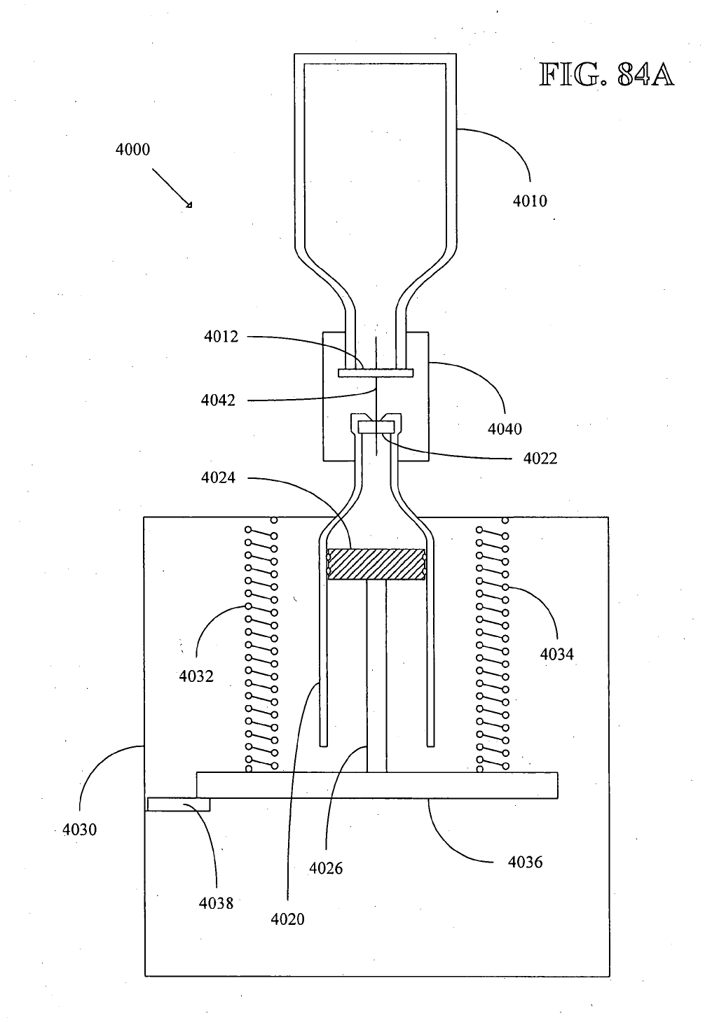

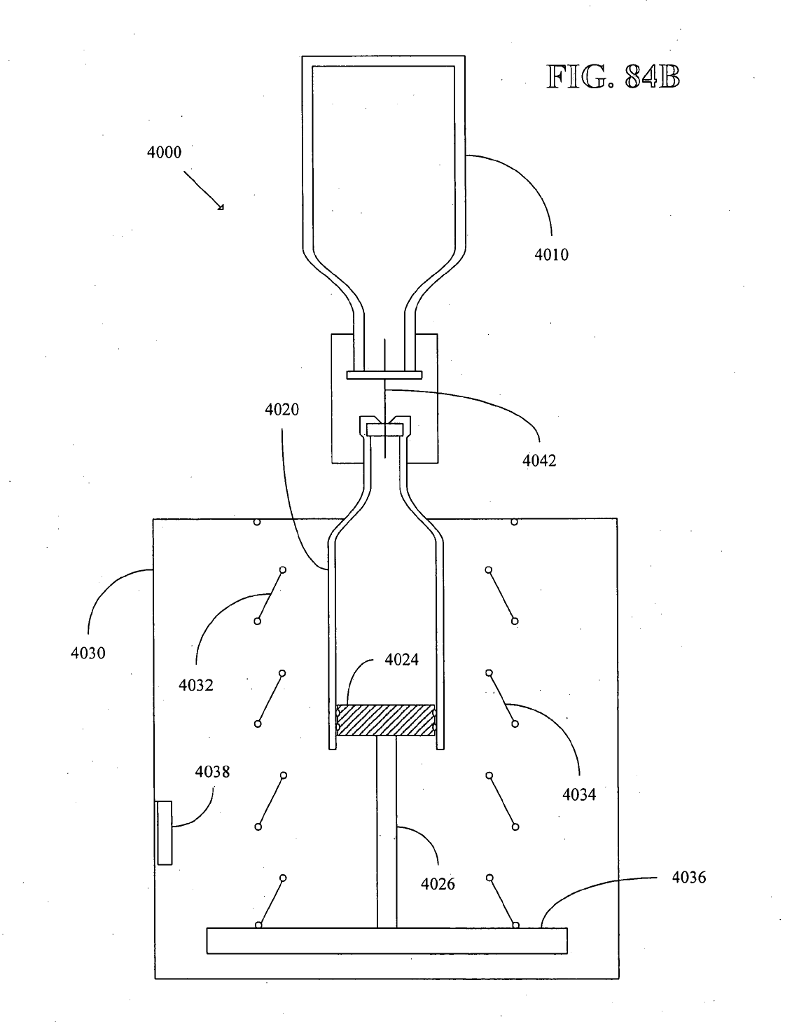

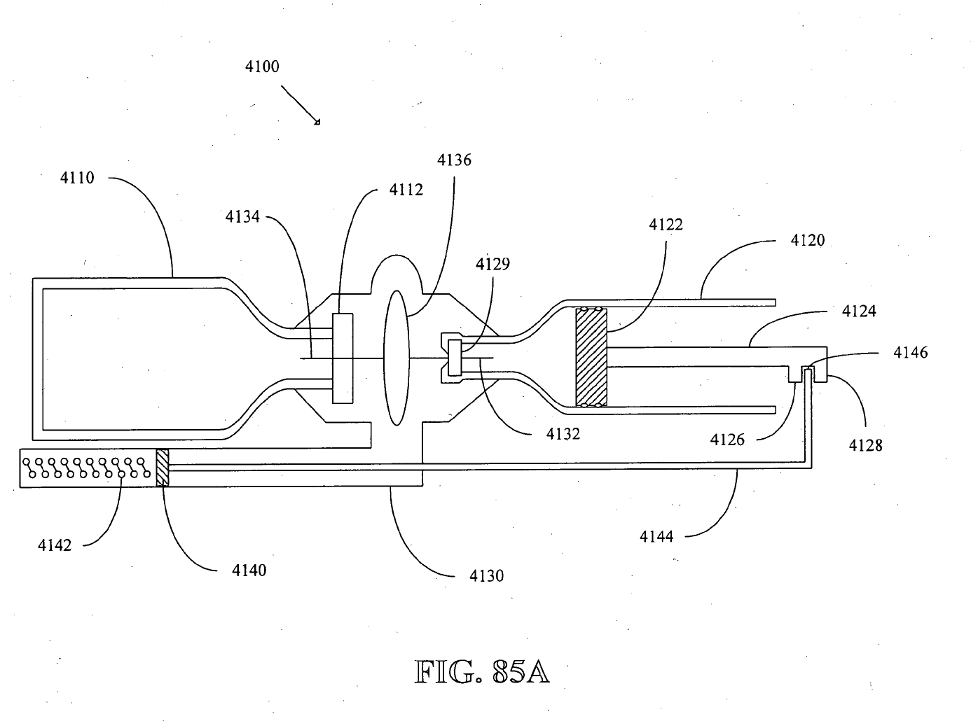

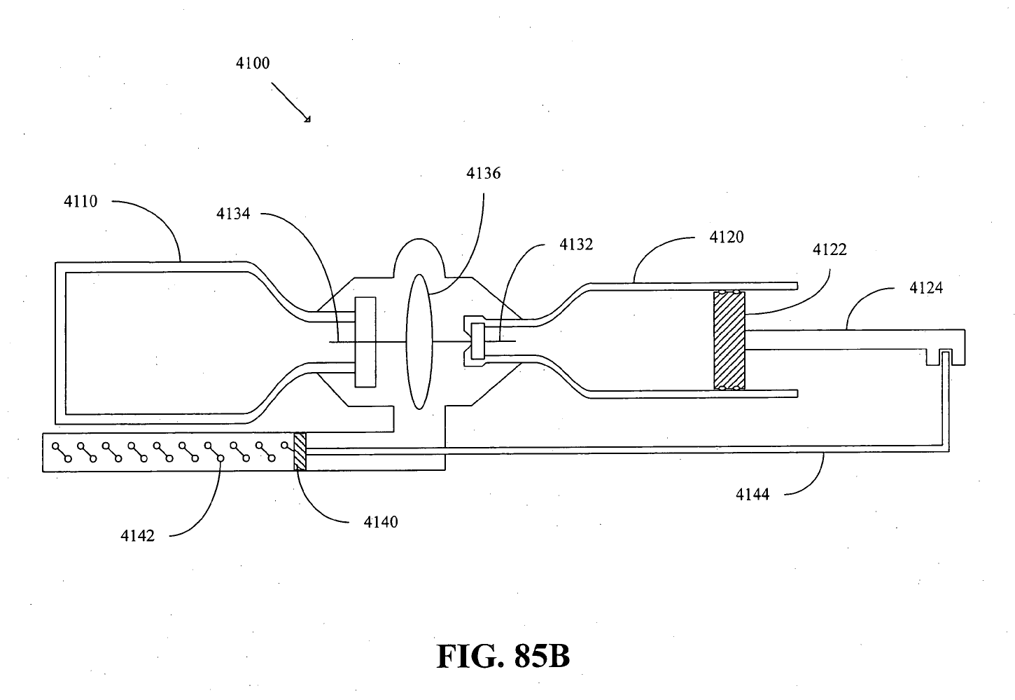

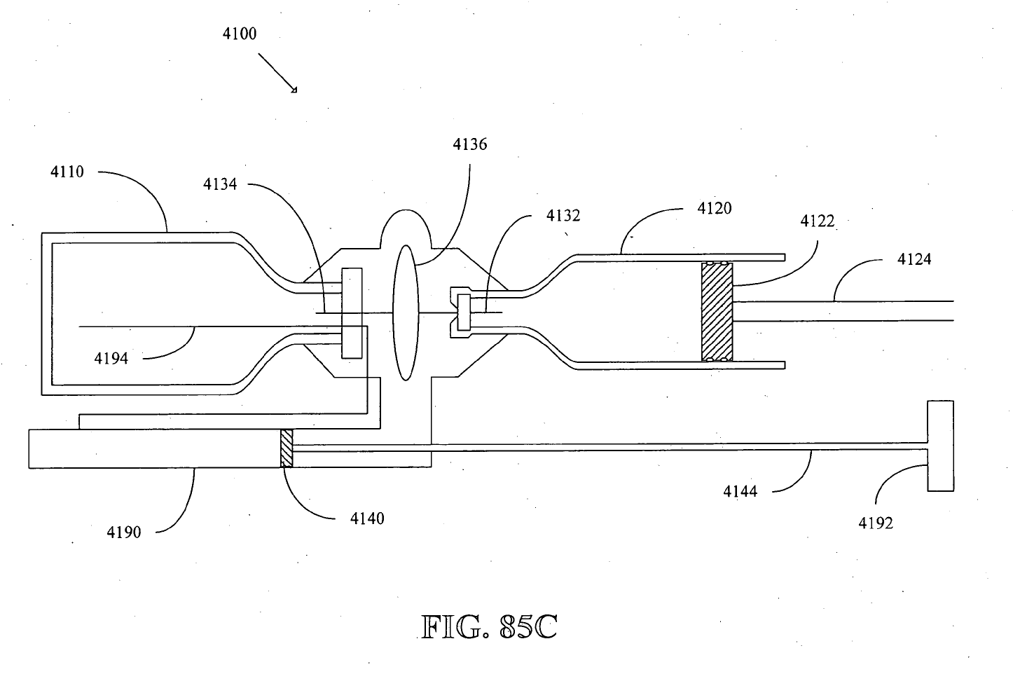

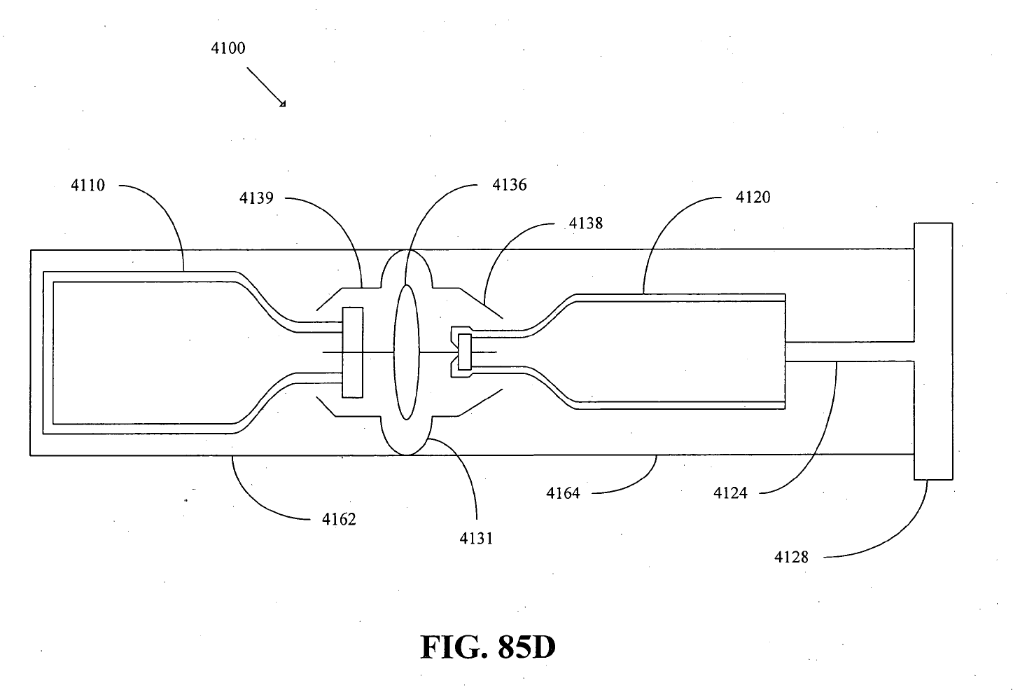

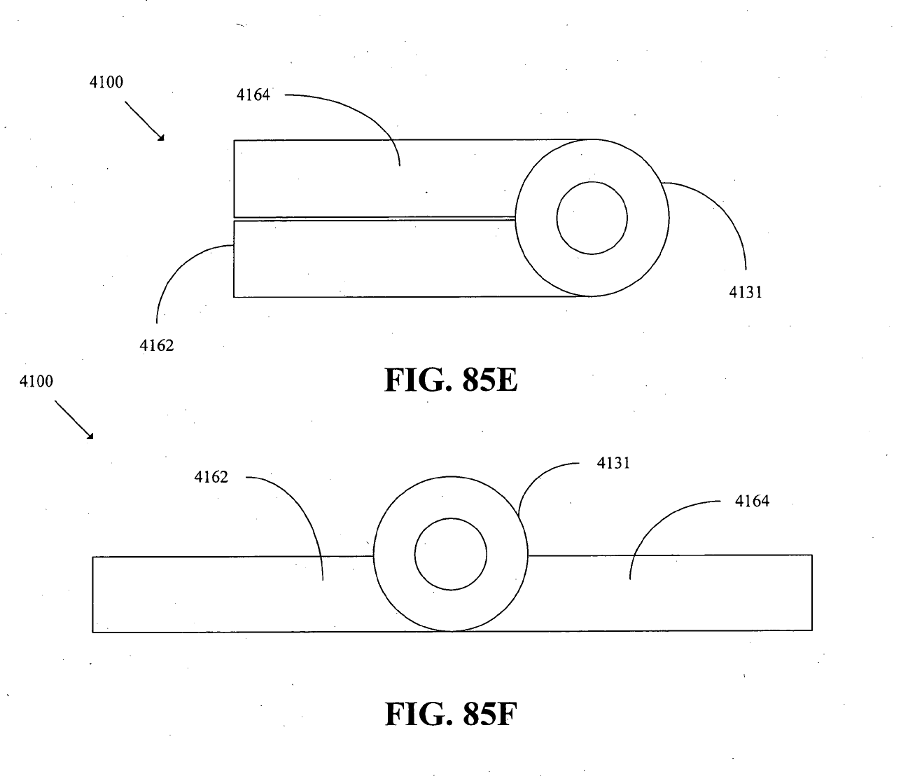

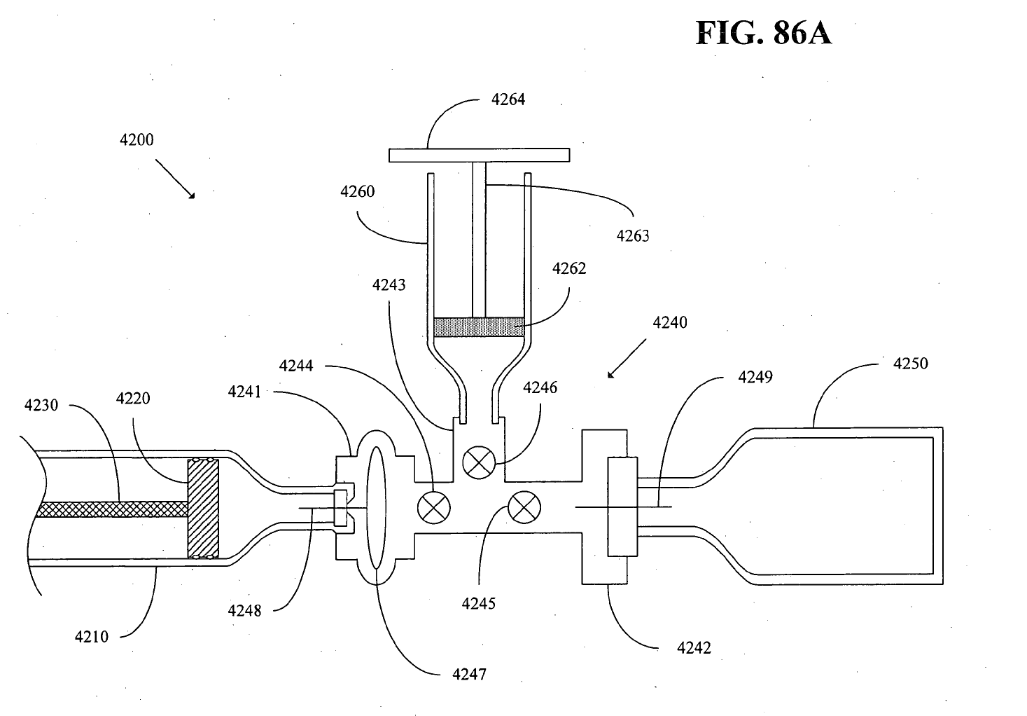

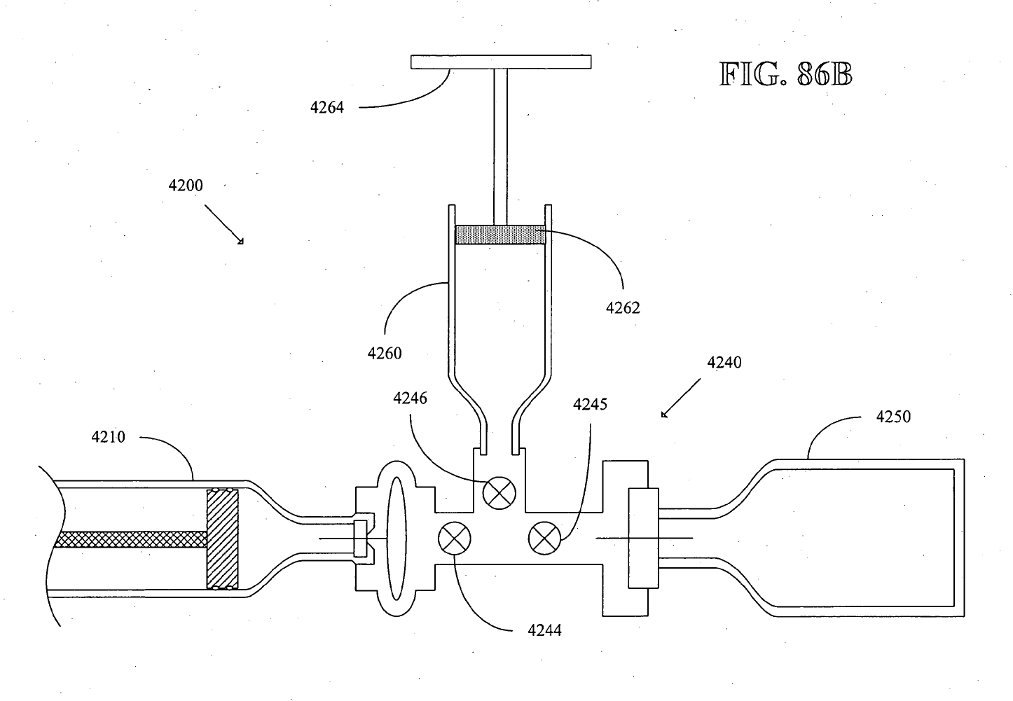

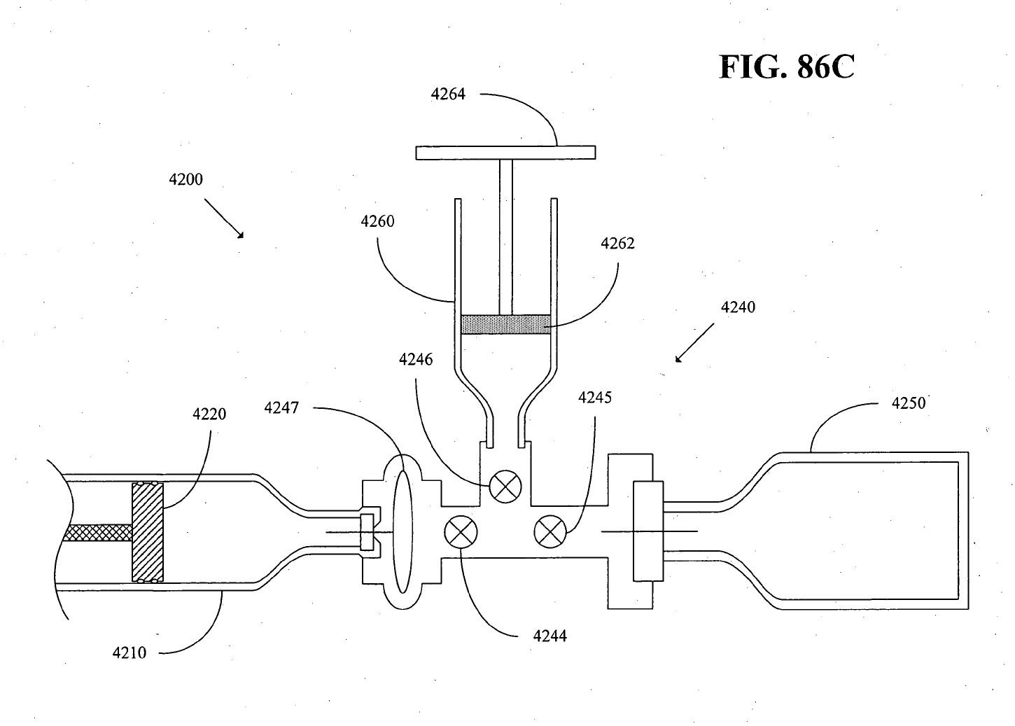

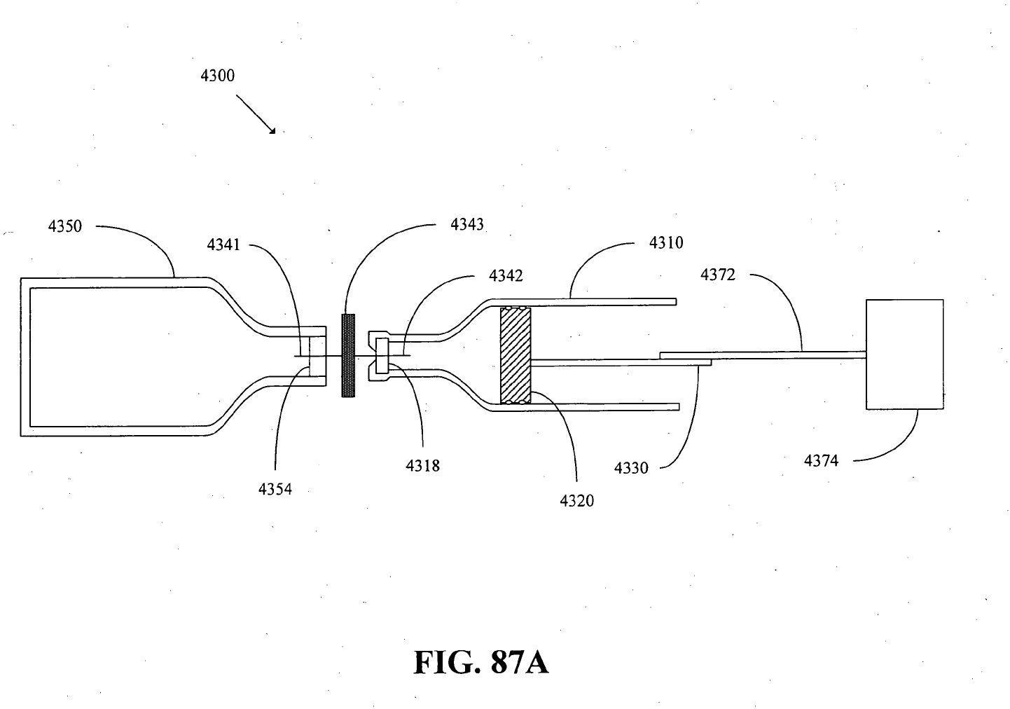

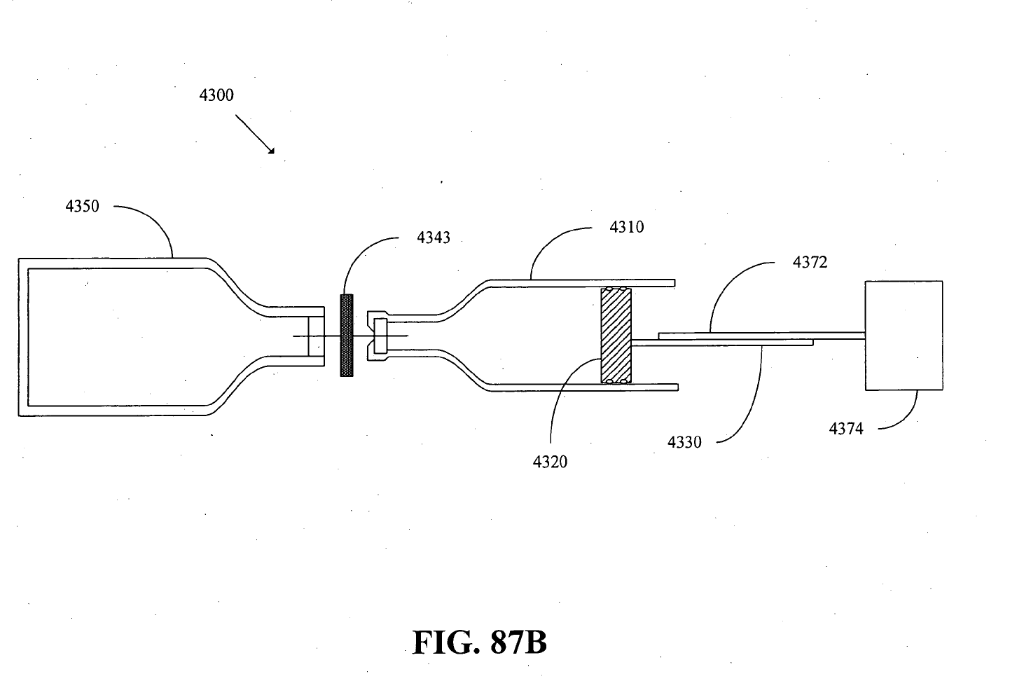

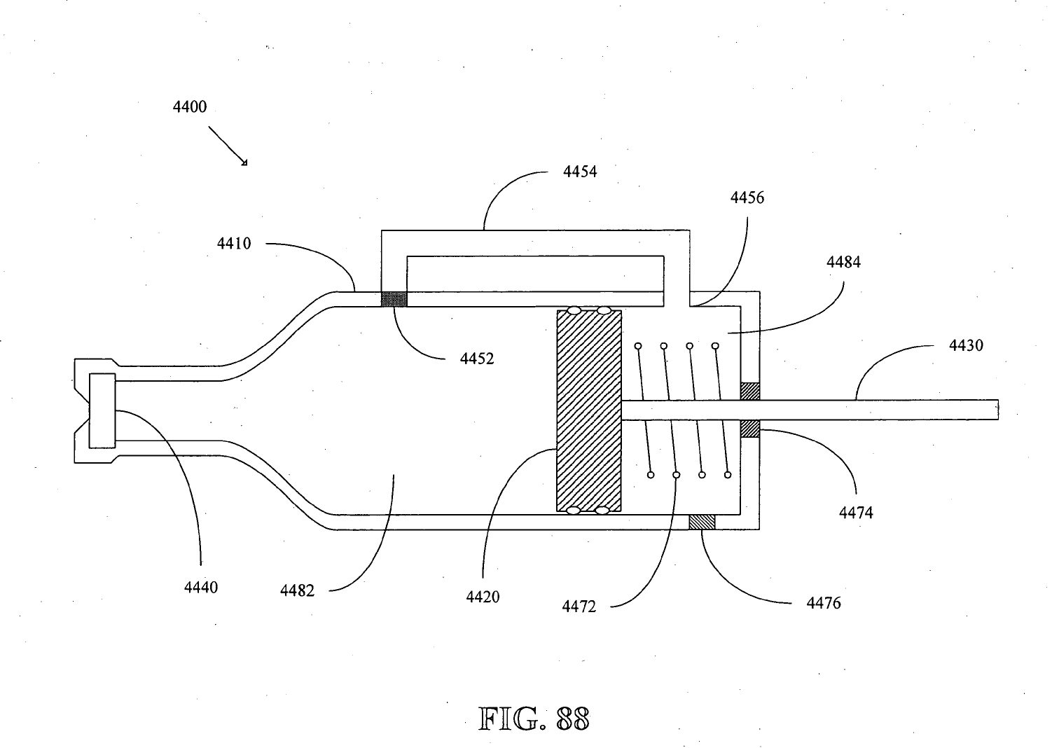

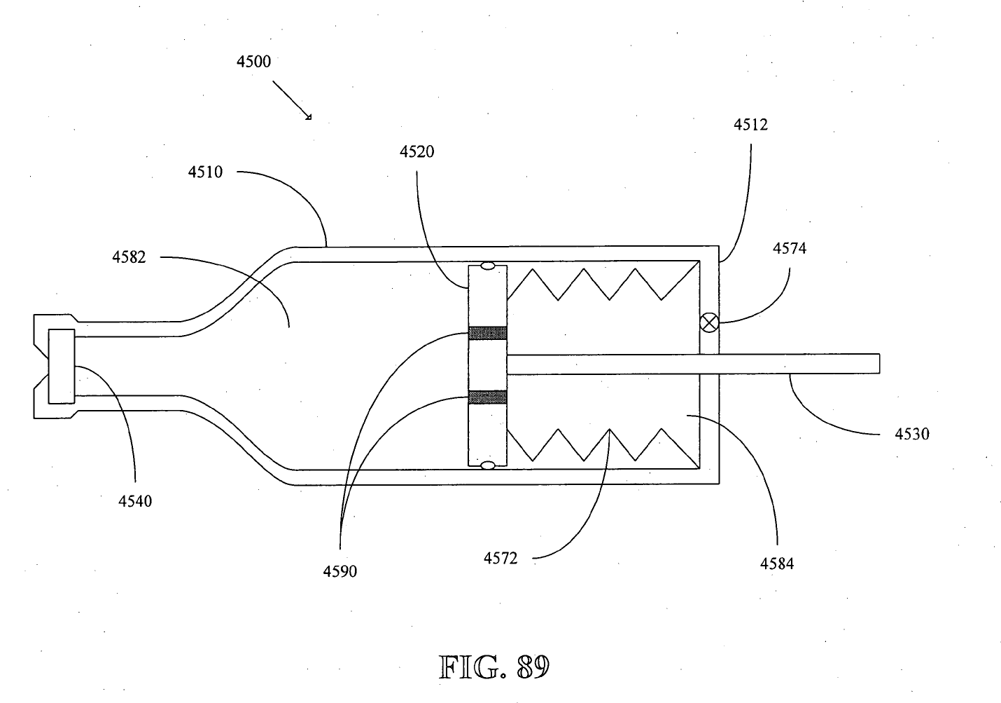

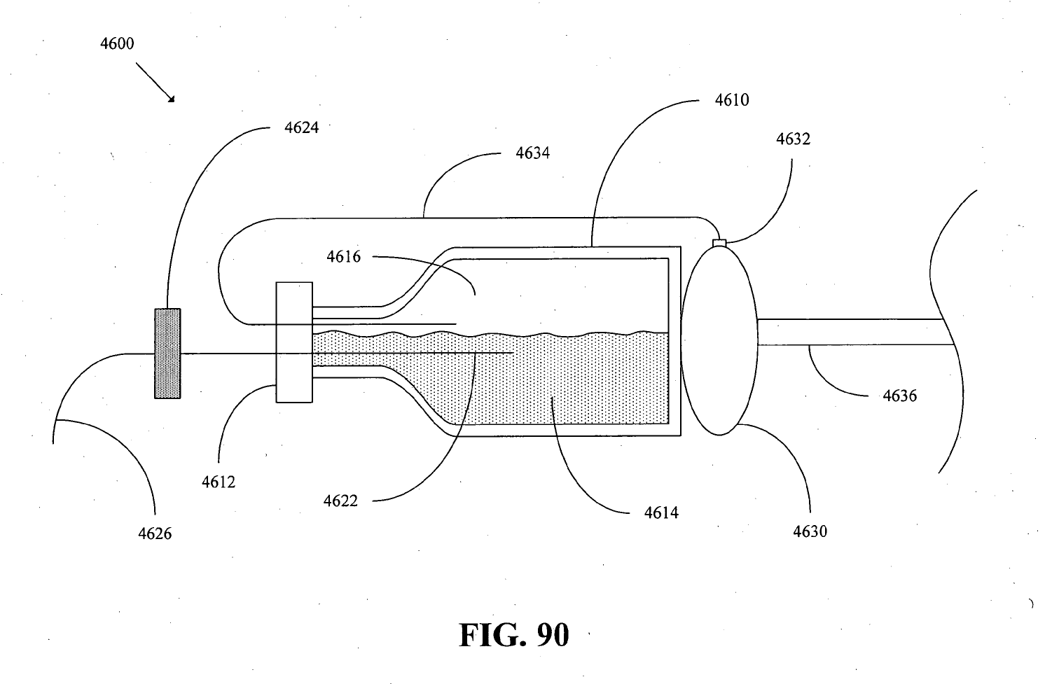

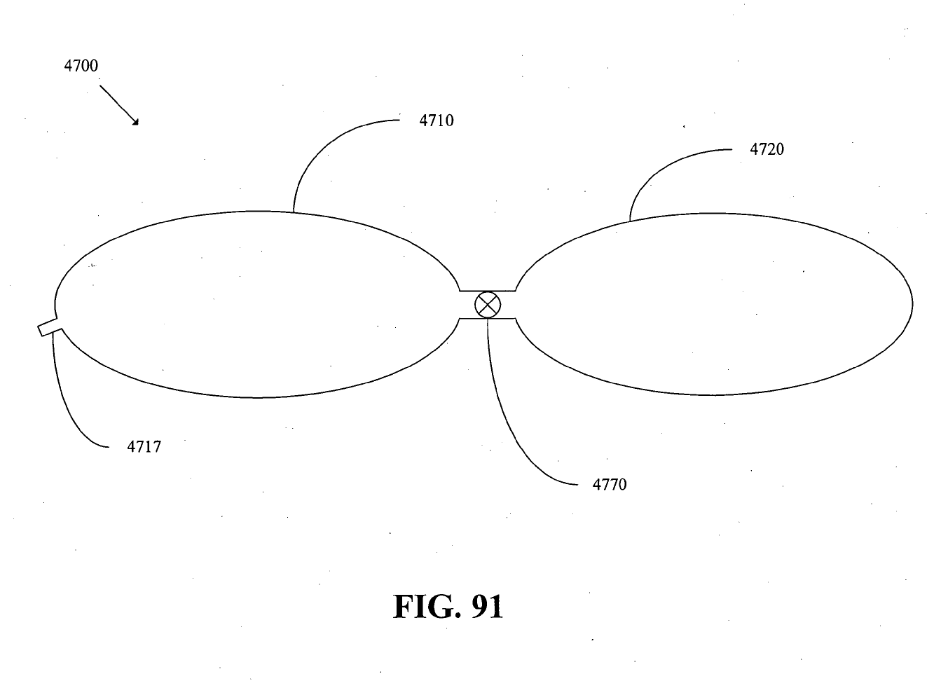

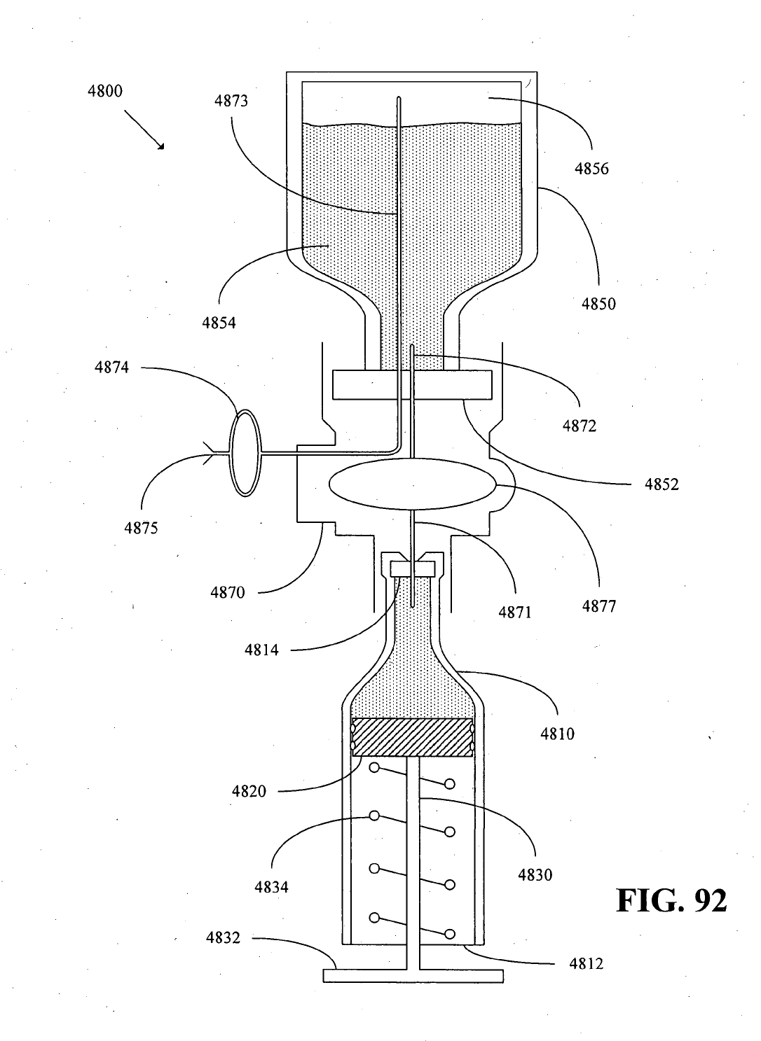

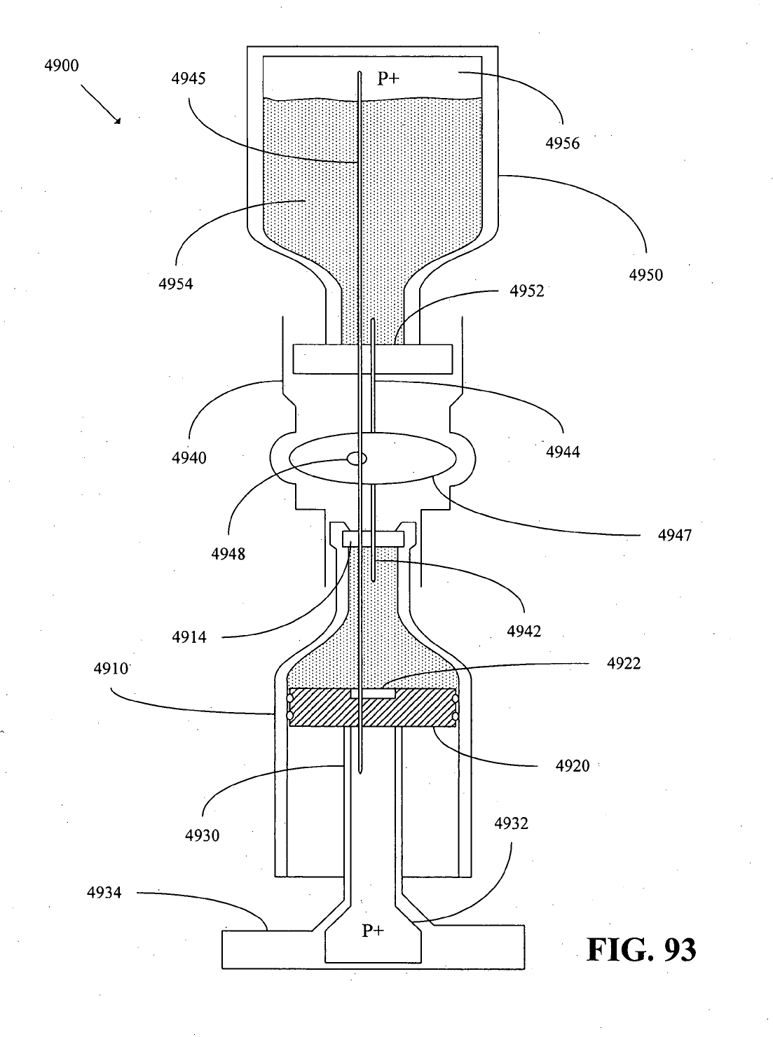

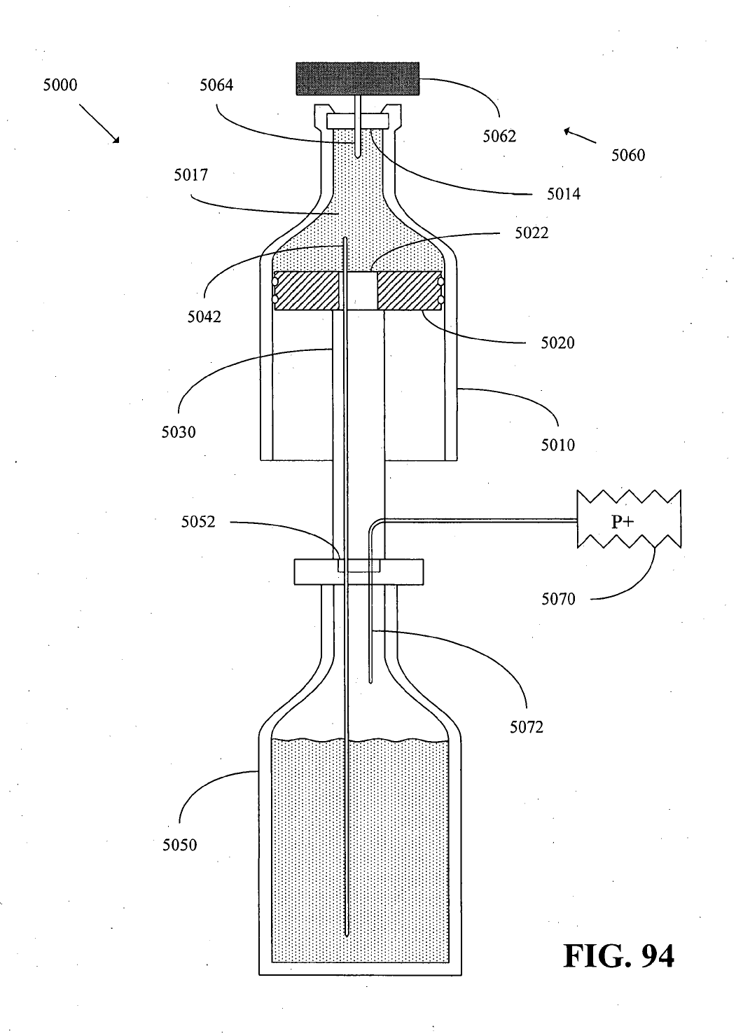

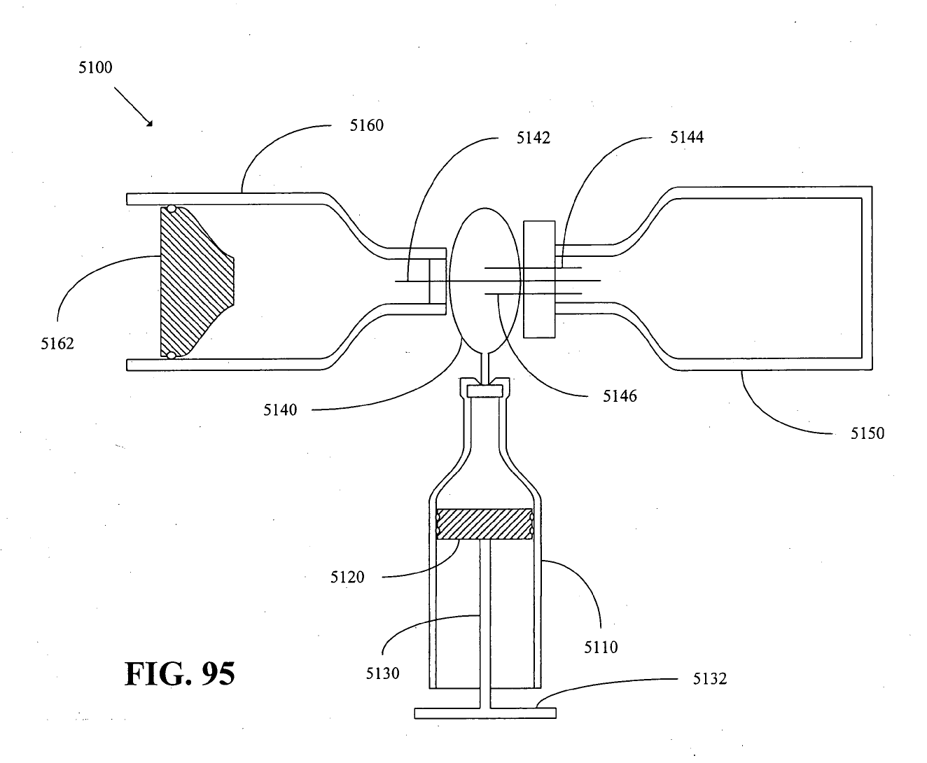

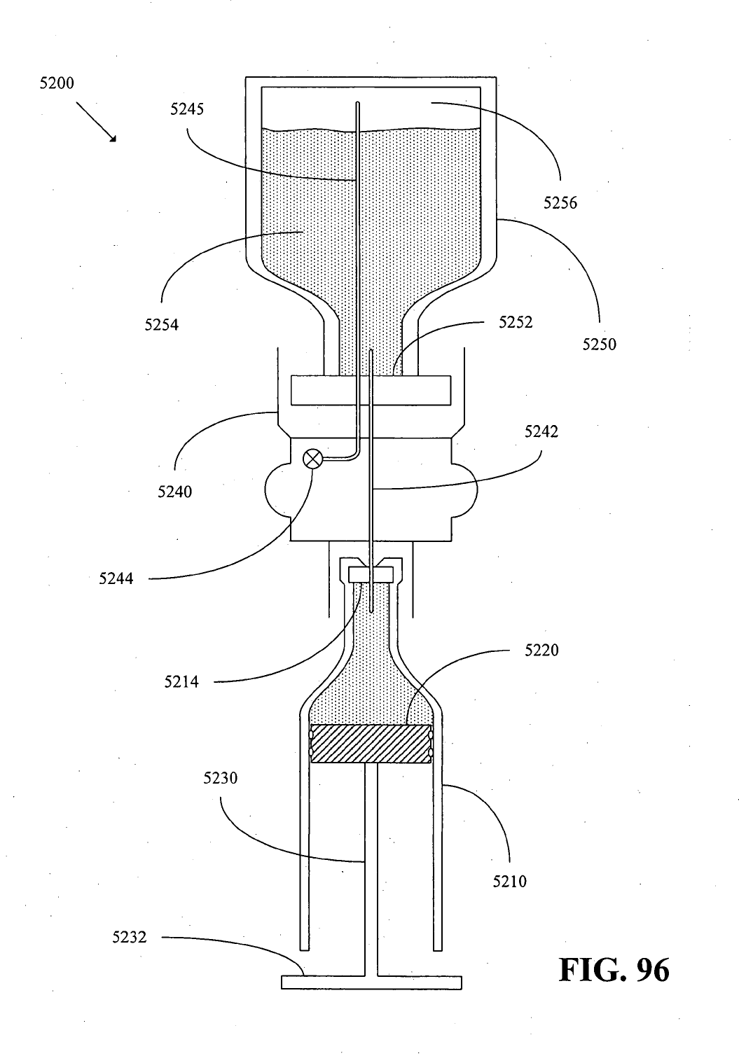

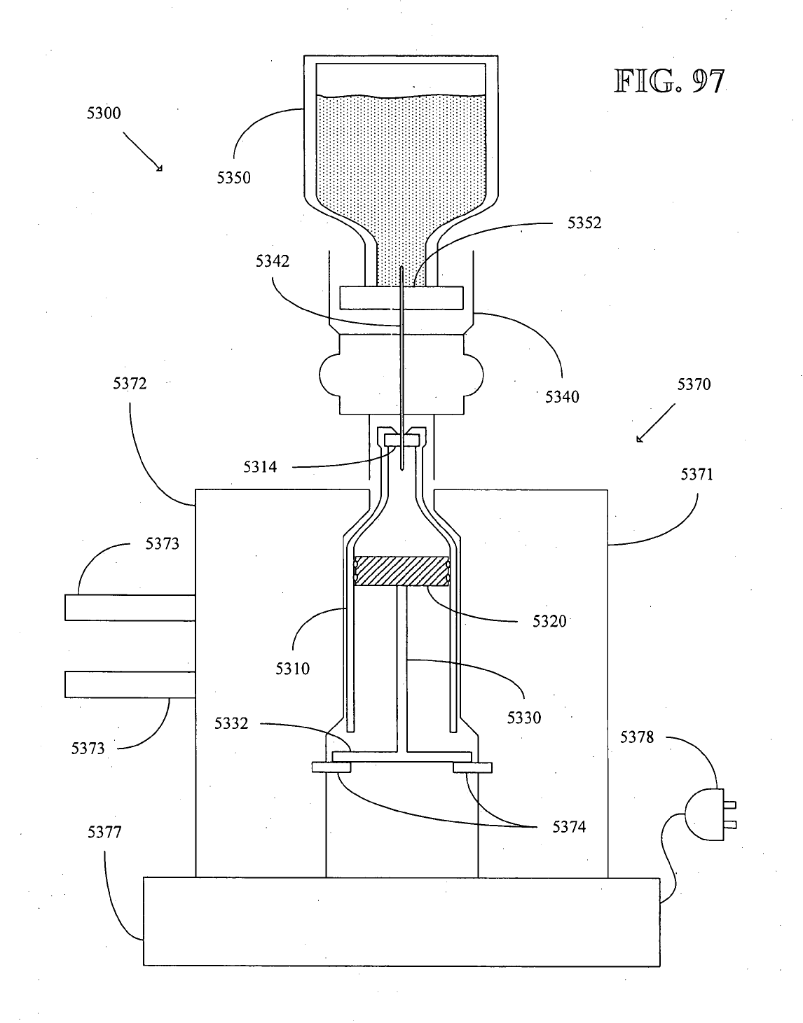

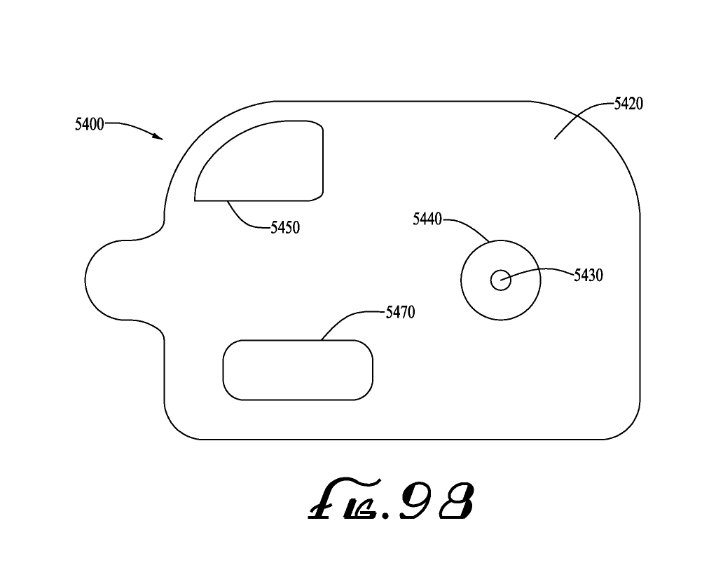

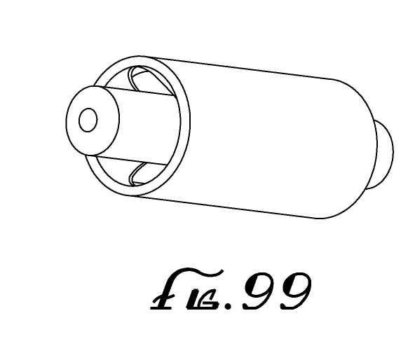

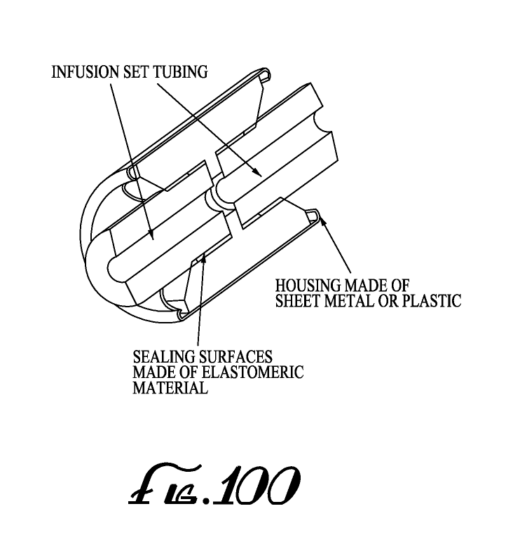

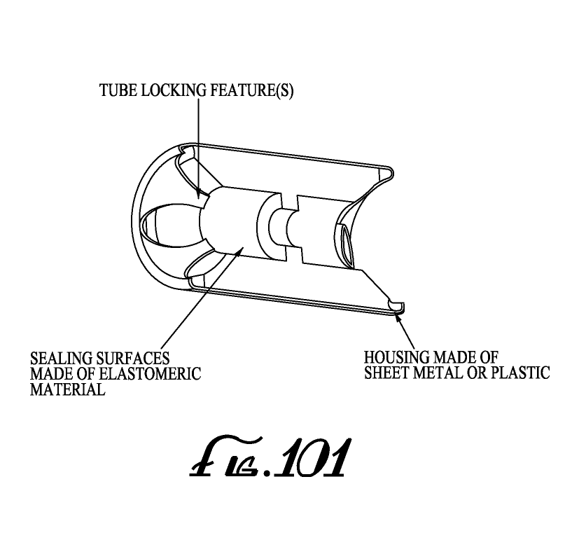

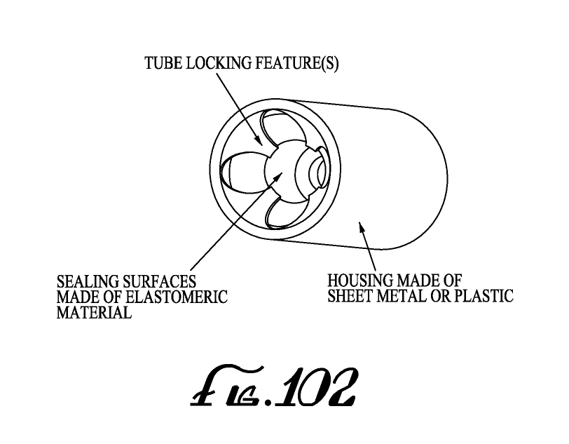

### Embodiments of FIGS. 74-79

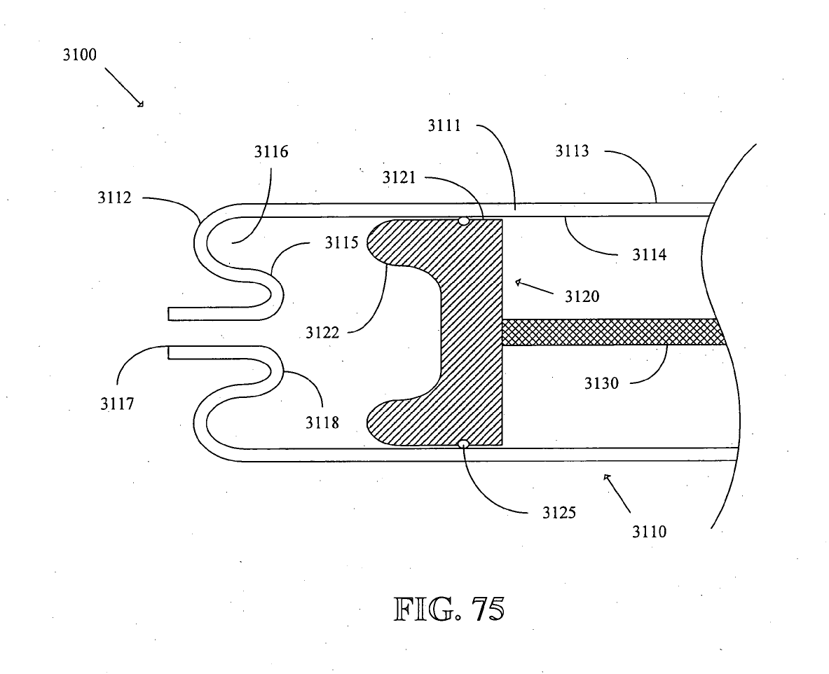

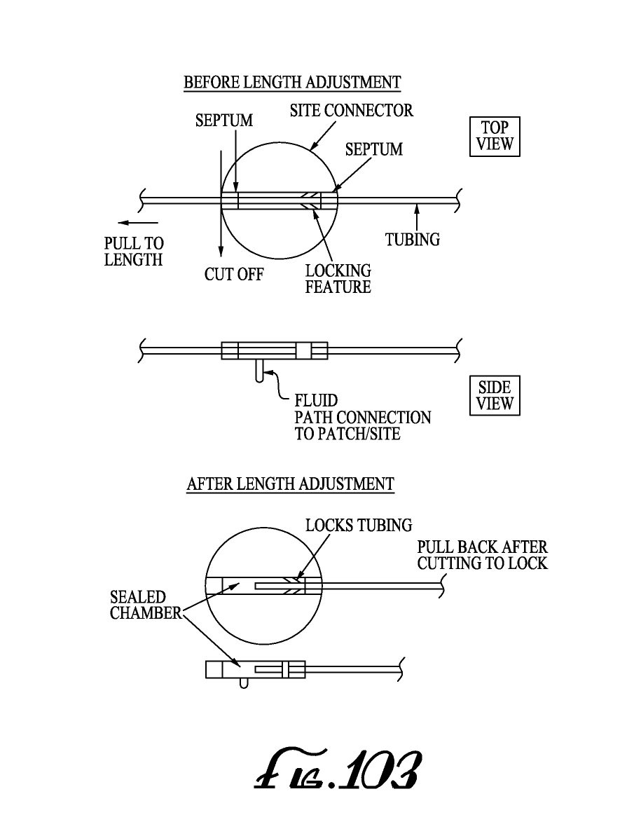

An embodiment of a coupling device for coupling fluid flow tubing ends together is shown in FIGS. 75-79. An embodiment of an adjustable length tubing for an infusion set is shown in FIG. 79. Such embodiments may be employed with infusion delivery devices and needle inserting devices as described herein or in other suitable systems.

While various embodiments of the present invention may be used with in an insulin delivery system for treating diabetes, other embodiments of the invention may be employed for delivering other infusion media to a patient-user for other purposes. For example, further embodiments of the invention may be employed for delivering other types of drugs to treat diseases or medical conditions other than diabetes, including, but not limited to drugs for treating pain or certain types of cancers, pulmonary disorders or HIV. Further embodiments may be employed for delivering media other than drugs, including, but not limited to, nutritional media including nutritional supplements, dyes or other tracing media, saline or other hydration media, or the like. Also, while embodiments of the present invention are described herein for delivering or infusing an infusion medium to a patient-user, other embodiments may be configured to draw a medium from a patient-user.

1. A connecting structure for connecting a first member in fluid flow connection with a second member, the connecting structure comprising:
a receptacle structure provided on the first member and having an interior chamber and an opening into the interior chamber;
a first septum provided within the interior chamber, adjacent the opening of the receptacle structure;
a hollow needle supported within the interior chamber of the receptacle structure, the hollow needle having a sharp end with an first opening into which fluid may flow, the sharp end facing the first septum, the hollow needle having a second opening out of which fluid may flow;
a connection portion provided on the second member, the connection portion having a hollow interior chamber and an opening that opens to the hollow interior chamber, the connection portion having a size and shape suitable to be inserted at least partially into the opening of the receptacle structure;
a second septum supported by the connection portion of the second member in a position to cover the opening in the connection portion;
wherein, upon receiving the connection portion within the opening of the receptacle structure, the connection portion pushes the first septum toward the sharp end of the hollow needle, and the sharp end of the hollow needle is caused to pierce the first septum and the second septum to come into fluid flow communication with the hollow interior chamber of the connection portion.

2. A connecting structure as recited in claim 1, wherein
the second opening of the hollow needle is provided in fluid flow communication with a needle injection site channel; and the needle injection site channel has an opening that is connectable to an needle inserting device for receiving at least a portion of a needle from the needle inserting device.

3. A connecting structure as recited in claim 1, further comprising a bias mechanism arranged to apply a bias force on the first septum directed toward the opening of the receptacle structure, wherein upon receiving the connection portion within the opening of the receptacle structure, the connection portion pushes the first septum toward the sharp end of the hollow needle against the bias force of the bias mechanism.

4. A connecting structure as recited in claim 3, wherein the bias mechanism comprises a coil spring.

5. A connecting structure as recited in claim 3, wherein the bias mechanism comprises a coil spring and wherein the hollow needle extends through the coil spring.

6. A connecting structure as recited in claim 1, wherein the second member comprises a fluid reservoir and wherein the connection portion of the second member comprises a portion of the fluid reservoir.

7. A connecting structure as recited in claim 1, wherein the receptacle structure is fixed with respect to a base portion and wherein the connection portion of the second member is provided in a housing that is connectable to the base portion.

8. A connecting structure as recited in claim 2, wherein:
the receptacle structure is fixed with respect to a base portion;
the connection portion of the second member is provided in a housing that is connectable to the base portion; and
the housing includes a recess through which a needle inserting device may extend when connected to the opening of the needle injection site channel.

9. A connecting structure as recited in claim 1, wherein the needle injection site channel has an opening that is connected to a needle inserting device for receiving at least a portion of a needle from the needle inserting device, the needle inserting device comprising:
a needle inserter housing having an internal chamber and a longitudinal dimension;
a plunger arranged for movement within the internal chamber, in the direction of the longitudinal dimension of the needle inserter housing, from a first plunger position to a second plunger position;
a plunger bias mechanism for imparting a bias force on the plunger when the plunger is in the first plunger position, wherein the bias force is directed toward the second plunger position;
a needle connected to the plunger, for movement with the plunger;
a hollow cannula having a hollow interior and arranged with the needle extending through the hollow interior;
an insert structure arranged for movement within the internal chamber of the needle inserter housing with movement of the plunger from the first plunger position to the second plunger position, the insert structure including a third septum and a body through which the needle extends;
wherein, upon movement of the plunger from the first plunger position to the second plunger position, the needle, hollow cannula and insert structure are moved to an insert position with movement of the plunger to the second plunger position.

10. A connecting structure as recited in claim 9, wherein
the needle inserter housing has least one slot or groove;
the body of the insert structure includes a shaped portion that is configured to engage the at least one slot or groove, upon the insert structure being moved as the plunger moves from the first plunger position to the second plunger position.

11. A connecting structure as recited in claim 10, wherein
the needle inserter housing has a flexible wall portion in the vicinity of the at least one slot or groove; and
the body of the insert structure is arranged to engage and outwardly flex the flexible wall portion of the needle inserter housing, upon the insert structure being moved as the plunger moves from the first plunger position to the second plunger position.

12. A needle injector device for connection in fluid-flow communication with the inlet or outlet port of a reservoir, the needle injector comprising:
a housing having a generally cylindrical interior surface surrounding a generally cylindrical hollow interior volume, the generally cylindrical interior surface having a central axis, an inner circumference, a first groove forming a generally spiral path around at least a portion of the inner circumference and a second groove extending in a generally linear path that is generally parallel to the central axis;
a moveable cam member supported for movement within the interior volume of the housing between a retracted position and an extended position, the cam member having an outer cam portion for extending into the first and second grooves and an inner cam portion disposed within the outer cam portion, the inner cam portion for supporting a needle for movement with the cam member between the retracted position and extended position, the outer cam portion being rotatably connected to the inner cam portion, to rotate relative to the inner cam portion;
wherein, when the moveable cam member is in the retracted position, the moveable cam member is arranged relative to the generally cylindrical interior surface, to be selectively moved from the retracted position to the extended position, in a rotary direction around the central axis and simultaneously in a direction generally parallel to the central axis, while the outer cam portion follows the spiral path of the first groove; and
wherein, when the moveable cam member is in the extended position, the moveable cam member is arranged relative to the generally cylindrical interior surface, to be moved from the extended position toward the retracted position, in a generally linear direction that is generally parallel to the central axis, while the outer cam portion follows the generally linear path of the second groove.

13. A device according to claim 12, further comprising a guide structure for engaging the inner cam portion for inhibiting rotation of the inner cam portion, as the outer cam portion engages the first groove and is moved from a retracted position to an extended position.

14. A device according to claim 13, wherein the guide structure comprises at least one strut extending within the housing, in a direction generally parallel to the central axis, the at least one strut for engaging the inner cam portion.

15. A device according to claim 14, wherein the inner cam portion has at least one opening through which the at least one strut extends through.

16. A device according to claim 12, wherein the housing has a first end and a second end and wherein the generally linear path connects to the generally spiral path adjacent the first end and adjacent the second end of the housing.

17. A device according to claim 12, further comprising a needle having a shaft having a piercing end, the needle supported for movement with the cam member such that the piercing end of the needle extends out of the housing, when the cam member is in the extended position.

18. A device according to claim 12, wherein:
the housing has a base end for arranging adjacent a user's skin;
the needle shaft has a sufficient length and width relative to a hollow cannula to extend through the hollow cannula and to extend the piercing end of the needle out one end of the cannula, while supporting the cannula on the needle for movement with the needle between the retracted position and the extended position, and to leave the cannula in the extended position in a user's skin, when (i) the base end of the housing is arranged adjacent the user's skin, (ii) the moveable needle and cannula are in the extended position and (iii) the moveable cam member is moved from the extended position toward the retracted position, while the cam projection follows the generally linear path of the second groove.

19.-26. (canceled)
